SEQUENCE LISTING (37 sequences)

SEQ ID NO: 1 - protein, 413 aa, synthetic construct
  REGION 1..413: note = PHY-13594
SEAAPAGYHLDKAVILSRHGVRAPTKMTQTMRDVTPYTWPEWPVPLGYITPRGEHLVSLMGGFYRQYFQQQGLLSRDSCPTANDVYVWTDVNQRTRKTGEAFLAGLAPECDLTIHHQNDIKQVDPLFHPVKAGICSMDKTQVQQAVEKQAGMPIDKLNQHYRPELALMSNVLNFPKSPYCQRHSGEQPCDFANAFPSKLNISDDGNEVQLEGAVGLSSTLAEIFLLEYAQGMPVVAWGNIHSEQEWNDLLKLHNAQFDLMERTPYIAKHNGTPLLQTIVNALNSNTTESKLPDISPDVKILFLAGHDTNIANIGGMLGMTWTLPGQPDNTPPGGALLFELWSDKEGTQYVSVKMVYQTLAQLRNQTPLTLKEPAGSVPLKIPGCDDQTAEGYCPLDTFTRLVNQVVEPACQLP

SEQ ID NO: 2 - protein, 413 aa, synthetic construct
  REGION 1..413: note = PHY-10931
SETAPAGYHLDKAVILSRHGVRAPTKMTQLMRDVTPNQWPEWPVPLGYITPRGEHLVSLMGGFYRQYFQQQGLLSRDRCPTANDVYVWTDVNQRTRKTGEAFLAGLAPQCPLTIHHQSNIKVVDPLFHPLKTGICSMDKTQVQQAVEKQAGMPIDKLNQYYAPELSLMSNVLNFPKSPYCRQHSVEKPCDFAQMFPSKLNISDDGNEVQLEGAVGLSSTLAEIFLLEHAQGMPVVAWGNIHSEQEWNDLLKLHNAQFDLMERTPYIAKHNGTPLLQTIVNALNSNTTARELPDISPDNKILFLAGHDTNIANIGGMLGMTWTLPGQPDNTPPGGALLFELWSDKAGTQYVSVHMVYQTLAQLRNQTPLTLKEPAGSVPLKIPGCDDQTAEGYCPLDTFTRLVNQVVEPACQLP

SEQ ID NO: 3 - protein, 413 aa, synthetic construct
  REGION 1..413: note = PHY-10957
SEAAPSGYHLQKAVILSRHGVRAPTKMTQLMRDVTPYTWPEWPVPLGYITPRGEHLVSLMGGFYRQWFQQQGILSKERCPTANDVYVWTDVNQRTRKTGEAFLAGLAPKCPLTIHHQSNIKVVDPLFHPLKTGICSMDKTQVQQAVEKQAGMPIDKLNQHYRPELALMSNVLNFPKSPYCRQHSVEKPCDFANAFPSKLNITDDGNEVQLEGAVGLSSTLAEIFLLEYAQGMPVVAWGNIHSEQEWNALLKLHNAYFDLMERTPYIAKHNGTPLLQTIVNALNSNTTARELPDISPDVKILFLAGHDTNIANIAGMLGMDWTLPGQPDNTPPGGALLFELWRDKEGTQYVSVKMVYQTLAQLRNQTPLTLKEPAGSVPLKIPGCDDQTAEGYCPLVTFTRLVNQVVEPACQIP

SEQ ID NO: 4 - protein, 413 aa, synthetic construct
  REGION 1..413: note = PHY-11569
SETAPSGYHLDKAVILSRHGVRAPTKMTQLMRDVTPYTWPEWPVPLGYITPRGEHLVSLMGGFYRQYFQQQGLLSKDNCPTANDVYVWTDVDQRTRKTGEAFLAGLAPECDLTIHHQNNIKVVDPMFHPLKTGICSMDKTQVQQAVEKQAGMPIDKLNQHYAPELALMSAVLNFPASPYCQQHSVDKPCDFANAFPSKLNITDDGNEVQLEGAVGLSSTLAEIFLLEYAQGMPVVAWGNIHSEQEWNALLKLHNAHFDLMSRTPYIAKHNGTPLLQTIVNALNSNTTARELPDISPDNKILFLAGHDTNIANIGGMLGMDWTLPGQPDNTPPGGALLFELWRDKAGTQYVSVHMVYQTLAQLRNQTPLTLKEPAGTVPLKIPGCDDQTAEGYCPLVTFTRLVNQSVEPACQLP

SEQ ID NO: 5 - protein, 413 aa, synthetic construct
  REGION 1..413: note = PHY-11658
SEAAPSGYHLDKAVLLSRHGVRAPTKMTQLMRDVTPYTWPEWPVPLGYITPRGEHLVSLMGGFYRQWFQQQGLLSKDRCPTANDVYVWADVGQRTRKTGEAFLAGLAPECDLTIHHQNDIKQVDPMFHPLKAGICSMDKTQVQQAVEKQAGMPIDKLNQHYRPELALMSNVLNFPKSPYCRQHSVEKPCDFAQMFPSKLNITDDGNEVQLEGAVGLSSTLAEIFLLEYAQGMPVVAWGNIHSEQEWNDLLKLHNAQFDLMQRTPYIAKHNGTPLLQTIVNALNSNTTARELPDISPDVKILFLAGHDTNIANIGGMLGMDWTLPGQPDNTPPGGALLFERWSDKDGTQYVSVHMVYQTLAQLRNQTPLTLKEPAGTVPLKIPGCDDQTAEGYCPLVTFTRLVNQSVEPACQLP

SEQ ID NO: 6 - protein, 413 aa, synthetic construct
  REGION 1..413: note = PHY-11673
SEAAPSGYHLDKAVILSRHGVRAPTKMTELMRDVTPYTWPEWPVPLGYITPRGEHLVSLMGGFYRQYFQQQGLLSKDRCPTANDVYVWTDVNQRTRKTGEAFLAGLAPECPLTIHHQSNIKVVDPLFHPVKTGLCSMDKTQVQQAVEKQAGMPIDKLNDKYAPELALMSNVLNFPKSPYCRQHSVEKPCDFAQMFPSKLSIGDDGNEVQLEGAVGLSSTLAEIFLLEYAQGMPVVAWGNIHSEQEWNDLLKLHNAHFDLMERTPYIAKHNGTPLLQTIVNALNSNTTESKLPDISPSVKILFLAGHDTNIANIGGMLGMDWTLPGQPDNTPPGGALLFELWRDKDGTQYVSVHMVYQTLAQLRNQTPLTLKEPAGTVPLKIPGCDDQTAEGYCPLVTFTVLVNQSVEPACQLP

SEQ ID NO: 7 - protein, 413 aa, synthetic construct
  REGION 1..413: note = PHY-11680
SETAPSGYHLDKAVILSRHGVRAPTKMTQLMRDVTPYTWPEWPVPLGYITPRGEHLVSLMGGFYRQYFQQQGLLSKDNCPTANDVYVWTDVDQRTRKTGEAFLAGLAPECDLTIHHQNNIKVVDPLFHPLKTGICSMDKTQVQQAVEKQAGMPIDKLNDHYRPELSLMSNVLNFPKSPYCQRHSGEKTCDFANAFPSKLIITDDGNKVALEGAVGLSSTLAEIFLLEYAQGMPKVAWGNIHSEQEWNDLLKLHNAQFDLMERTPYIAKHNGTPLLQTIVNALNSNTTARELPDISPDVKILFLAGHDTNIANIGGMLGMDWTLPGQPDNTPPGGALLFELWRDKEGTQYVSVKMVYQTLAQLRNQTPLTLKEPAGSVPLKIPGCDDQTAEGYCPLDTFTRLVNQVVEPACQIP

SEQ ID NO: 8 - protein, 413 aa, synthetic construct
  REGION 1..413: note = PHY-11895
SEAAPSGYHLDKAVLLSRHGVRAPTKMTQLMRDVTPYTWPEWPVPLGYITPRGEHLVSLMGGFYRQWFQQQGLLSRDRCPTANDVYVWTDVNQRTRKTGEAFLAGLAPECDLTIHHQKNIKVVDPLFHPLKAGICSMDKTQVQQAVEKQAGMPIDKLNQHYAPELSLMSNVLNFPKSPYCQRHSGEKTCDFANAFPSKLNISDDGNEVQLEGAVGLSSTLAEIFLLEYAQGMPVVAWGNIHSEQEWNALLKLHNAHFDLMERTPYIAKHNGTPLLQTIVNALNSNTTARELPDISPSVKILFLAGHDTNIANIGGMLGMTWTLPGQPDNTPPGGALLFELWSDKAGTQYVSVHMVYQTLTQLRNQTPLTLKEPAGSVPLKIPGCDDQTAEGYCPLDTFTRLVNQVVEPACQLP

SEQ ID NO: 9 - protein, 413 aa, synthetic construct
  REGION 1..413: note = PHY-11932
SETAPAGYHLDKAVILSRHGVRAPTKMTQTMRDVTPYTWPEWPVPLGYITPRGEHLVSLMGGFYRQYFQQQGLLSAERCPTANDVYVWTDVNQRTRKTGEAFLAGLAPQCPLTIHHQNDIKQVDPMFHPLKAGICSMDKTQVQQAVEKQAGMPIDKLNDHYRPELSLMSNVLNFPKSPYCARHSGDKPCDFAQMMPSKLNITDDGNEVQLEGAVGLSSTLAEIFLLEYAQGMPVVAWGNIHSEQEWNALLKLHNAQFDLMERTPYIAKHNGTPLLQTIVNALNSNTTARELPDISPDVKILFLAGHDTNIANIAGMLGMTWTLPGQPDNTPPGGALLFELWSDKAGTQYVSVHMVYQTLAQLRNQTPLTLKEPAGSVPLKIPGCDDQTAEGYCPLDTFTRLVNQVVEPACQLP

SEQ ID NO: 10 - protein, 413 aa, synthetic construct
  REGION 1..413: note = PHY-12058
SEAAPAGYHLDKVVILSRHGVRAPTKMTQTMRDVTPYTWPEWPVPLGYITPRGEHLVSLMGGFYRQKFQQQGLLSKDRCPTANDVYVWTDVDQRTRKTGEAFLAGLAPKCPLTIHHQSNIKVVDPLFHPLKTGICSMDKTQVQQAVEKQAGMPIDKLNQHYRPELSLMSNVLNFPKSPYCAQHSGEKPCDFAQMFPSKLNISDDGNEVQLEGAVGLSSTLAEIFLLEYAQGMPVVAWGNIHSEQEWNDLLKLHNAYFDLMERTPYIAKHNGTPLLQTIVNALNSNTTARELPDISPSVKILFLAGHDTNIANIGGMLGMDWTLPGQPDNTPPGGALLFELWRDKDGTQYVSVKMVYQTLAQLRNQTPLTLKEPAGSVPLKIPGCDDQTAEGYCPLDTFTRLVNQSVEPACQIP

SEQ ID NO: 11 - protein, 413 aa, synthetic construct
  REGION 1..413: note = PHY-12663
SEAAPSGYHLDKVVILSRHGVRAPTKMTQLMRDVTPYTWPEWPVPLGYITPRGEHLVSLMGGFYRQWFQQQGLLSKDRCPTANDVYVWTDVNQRTRKTGEAFLAGLAPQCPLTIHHQKNIKVVDPLFHPLKTGICSMDKTQVQQAVEKQAGMPIDKLNQHYRPELALMSNVLNFPKSPYCQRHSGDEPCDFANAFPSKLNITDDGNEVQLEGAVGLSSTLAEIFLLEYAQGMPVVAWGNIHSEQEWNDLLKLHNAQFDLMERTPYIAKHNGTPLLQTIVNALNSNTTARELPDISPDNKILFLAGHDTNIANIGGMLGMDWTLPGQPDNTPPGGALLFERWRDKEGTQYVSVHMVYQTLAQLRNQTPLTLKEPAGSVPLKIPGCDDQTAEGYCPLDTFTRLVNQVVEPACQIP

SEQ ID NO: 12 - protein, 413 aa, synthetic construct
  REGION 1..413: note = PHY-12784
SEAAPSGYHLDKAVLLSRHGVRAPTKMTQLMRDVTPNQWPEWPVPLGYITPRGEHLVSLMGGFYRQYFQQQGILSKDSCPTANDVYVWTDVNQRTRKTGEAFLAGLAPQCPLTIHHQSDIKQVDPMFHPLKAGICSMDKTQVQQAVEKQAGMPIDKLNQYYAPELALMSNVLNFPKSPYCQRHSGEQTCDFAQMFPSKLNISDDGNEVQLEGAVGLSSTLAETFLLEYAQGMPVVAWGNIHSEQEWNALLKLHNAQFDLMQRTPYIAKHNGTPLLQTIVNALNSNTTARELPDISPSVKILFLAGHDTNIANIGGMLGMDWTLPGQPDNTPPGGALLFELWRDKEGTQYVSVHMVYQTLTQLRNQTPLTLKEPAGTVPLKIPGCDDQTAEGYCPLVTFTRLVNQVVEPACQIP

SEQ ID NO: 13 - protein, 413 aa, synthetic construct
  REGION 1..413: note = PHY-13177
SEAAPSGYHLDKVVILSRHGVRAPTKMTQLMRDVTPNQWPEWPVPLGYITPRGEHLVSLMGGFYRQYFQQQGLLSKDRCPTANDVYVWADVNQRTRKTGEAFLAGLAPRCDLTIHHQKDIKQVDPMFHPLKAGICSMDKTQVQQAVEKQAGMPIDKLNQHYRPELALMSNVLNFPKSPYCAQHSGEKPCDFAQMFPSKLNISDDGNEVQLEGAVGLSSTLAEIFLLEYAQGMPVVAWGNIHSEQEWNDLLKLHNAQFDLMQRTPYIAKHNGTPLLQTIVNALNSNTTARELPDISPDNKILFLAGHDTNIANIGGMLGMDWTLPGQPDNTPPGGALLFERWRDKAGTQYVSVHMVYQTLAQLRNQTPLTLKEPAGTVPLKIPGCDDQTAEGYCPLDTFTRLVNQVVEPACQLP

SEQ ID NO: 14 - protein, 413 aa, synthetic construct
  REGION 1..413: note = PHY-13371
SEAAPAGYHLQKAVILSRHGVRAPTKMTQTMRDVTPYTWPEWPVPLGYITPRGEHLVKLMGGFYRQYFQQQGLLSRDRCPTANDVYVWTDVNQRTRKTGEAFLAGLAPECDLTIHHQSDIKQVDPLFHPVKAGICSMDKTQVQQAVEKQAGMPIDKLNQHYRPELALMSNVLNFPKSPYCQQHSVEQPCDFANAFPSYLNISDDGNEVQLEGAVGLGSTLAEIFLLEYAQGMPVVAWGNIHSEQEWNALLKLHNAHFNLMERTPYIAKHNGTPLLQTIVNALNSNTTARELPDISPDVKILFLAGHDTNIANIGGMLGMTWTLPGQPDNTPPGGALLFELWSDKEGTQYVAVKMVYQTLAQLRNQTPLTLKEPAGTVPLKIPGCDDQTAEGYCPLDTFTRLVNQVIEPACQLP

SEQ ID NO: 15 - protein, 413 aa, synthetic construct
  REGION 1..413: note = PHY-13460
SEAAPSGYHLQKAVILSRHGVRAPTKMTQLMRDVTPYTWPEWPVPLGYITPRGEHLVKLMGGFYRQYFQQQGLLSRDNCPTANDVYVWTDVAQRTRKTGEAFLAGLAPECDLTVHHQNDIKQVDPLFHPVKAGICSMNKSQVQQAVEKQAGMPIDKLNQHYRPELALMSNVLNFPKSPYCQRHSGEKPCDFAQMMPSKLNISDDGNEVQLEGAVGLSSTLAEIFLLEYAQGMPVVAWGNIHSEQEWNALLKLHNAQFDLMERTPYIAKHNGTPLMQTIVNALNSNTTARELPDISPDVKILFLAGHDTNIANIAGMLGMTWTLPGQPDNTPPGGALLFELWSDKEGTQYVSVKMVYQTLAQLRNQTPLTLKEPAGTVPLKIPGCDDQTAEGYCPLDTFTRLVNQVVEPACQLP

SEQ ID NO: 16 - protein, 413 aa, synthetic construct
  REGION 1..413: note = PHY-13513
SEAAPSGYHLQKAVILSRHGVRAPTKMTQLMRDVTPYTWPEWPVPLGYITPRGEHLVKLMGGFYRQYFQQQGLLSRDSCPTANDVYVWTDVNQRTRKTGEAFLAGLAPGCDLTIHHQSDIKQADPLFHPVKAGICSMDKTQVQQAVEKQAGGPIETLNQRYQAELALMSSVLDFPKSPYCQRHSVDKPCDFANAFPSYLNISDDGNEVQLEGAVGLSSTLAEIFLLEYAQGMPVVAWGNIHSEQEWNALLKLHNAHFDLMERTPYIAKHNGTPLLQTIVNALNSNTTARELPDISPDVKILFLAGHDTNIANIGGMLGMTWTLPGQPDNTPPGGALLFELWRDKDGTQYVSVHMVYQTLAQLRNMTPLTLKEPAGSVPLKIPGCDDQTAEGYCPLDTFTRLVNQVVEPACQLP

SEQ ID NO: 17 - protein, 413 aa, synthetic construct
  REGION 1..413: note = PHY-13637
SEAAPAGYHLQKAVILSRHGVRAPTKMTQLMRDVTPYTWPEWPVPLGYITPRGEHLVSLMGGFYRQYFQQQGLLSKDNCPTANDVYVWTDVNQRTRKTGEAFLAGLAPECDLTIHHQNDIKQVDPLFHPLKAGICSMDKTQVQQAVEKQAGMPIDKLNQHYRPELALMSSVLDFPKSPYCQHHSGDQPCDFAQMFPSYLNISDDGNEVQLEGAVGLSSTLAEIFLLEYAQGMPVVAWGNIHSEQEWNDLLKLHNAYFDLMERTPYIAKHNGTPLLQTIVNALNSNTTARELPDVSPDVKILFLAGHDTNIANIGGMLGMDWTLPGQPDNTPPGGALLFELWSDKEGTQYVSVKMVYQTLAQLRNQTPLTLKEPAGSVPLKIPGCDDQTAEGYCPLDTFTRLVNQVVEPACQLP

SEQ ID NO: 18 - protein, 413 aa, synthetic construct
  REGION 1..413: note = PHY-13705
SEAAPAGYHLDKAVILSRHGVRAPTKMTQLMRDVTPYTWPEWPVPLGYITPRGEHLVKLMGGFYRQYFQQQGLLSRDSCPTANDVYVWTDVNQRTRKTGEAFLAGLAPECDLTIHHQSDIKQADPLFHPLKAGICSMDKTQVQQAVEKQAGMPIDKLNQHYRAELALMSNVLNFPKSPYCRQHSVEQPCDFANAFPSRLNISDDGNEVQLEGAVGLSSTLAEIFLLEYAQGMPVVAWGNIHSEQEWNDLLKLHNAYFDLMERTPYIAKHQGTPLLQTIVNALNSNTTARELPDISPDVKILFLAGHDTNIANIAGMLGMDWTLPGQPDNTPPGGALLFELWSDKEGTQYVSVKMVYQTLAQLRNQTPLTLKEPAGSVPLKIPGCDDQTAEGYCPLDTFTRLVNRVVEPACQLP

SEQ ID NO: 19 - protein, 413 aa, synthetic construct
  REGION 1..413: note = PHY-13713
SEAAPAGYHLQKAVILSRHGVRAPTKMTQLMRDVTPYTWPEWPVPLGYITPRGEHLVSLMGGFYRQYFQQQGLLSRDRCPTPDAVYVWTDVNQRTRKTGEAFLAGLAPECDLTIHHQNDIHQVDPLFHPVKAGICSMDKTQVQQAVEKQAGMPIDKLNQHYRPELALMSNVLNFPKSPYCQRHSVDKPCDFAQMMPSYLNISDDGNEVQLEGAVGLSSTLAEIFLLEYAQGMPVVAWGNIHSEQEWNDLLKLHNAHFDLMERTPYIAKHQGTPLLQTIVNALNSNTTESKLPDISPDVKILFLAGHDTNIANIAGMLGMTWTLPGQPDNTPPGGALLFELWRDKAGTQYVSVKMVYQTLAQLRNQTPLTLKEPAGSVPLKIPGCDDQTAEGYCPLDTFTRLVNQVVEPACQLP

SEQ ID NO: 20 - protein, 412 aa, synthetic construct
  REGION 1..412: note = PHY-13747
SEAAPSGYHLQKAVILSRHGVRAPTKMTQLMRDVTPYTWPEWPVPLGYITPRGEHLVSLMGGFYRQYFQQQGLLSKDNCPTANDVYVWTDVNQRTRKTGEAFLAGLAPQCDLAIHHQNDIKQVDPLFHPLKAGICSMDKTQVQQAVEKQAGMPIDKLNQHYRPELALMSNVLNFPKSPYCRRHSGEKPCDFAQMMPSKLNISDDGNEVQLEGAVGLSSTLAEIFLLEYAQGMPVVAWGNIHSEEEWNDLLKLHNAYFDLMQRTPYIAKHNGTPLMQTISNALNPNATESKLPDISPDNKILFIAGHDTNIANIGGMLGMTWTLPGQPDNTPPGGALLFELWSDKEGTQYVSVKMVYQTLAQLRNQTPLTLKEPAGTVPLKIPGCDDQTAEGYCPLDTFTRLVNQVVEPACQI

SEQ ID NO: 21 - protein, 412 aa, synthetic construct
  REGION 1..412: note = PHY-13779
SEAAPAGYHLQKAVILSRHGVRAPTKMTQTMRDVTPHQWPEWPVPLGYITPRGEHLVKLMGGFYRQYFQQQGLLSKDRCPTANDVYVWTDVNQRTRKTGEAFLAGLAPECDLTIHHQSDIKQVDPLFHPLKAGICSMDKTQVQQAVEKQAGMPIDELNQHYRPELALMSNVLNFPKSPYCQRHSGEQPCDFANAFPSYLNISDDGNEVQLEGAVGLSSTLAEIFLLEYAQGMPVVAWGNIHSEQEWNDLLKLHNAYFDLMQRTPYIAKHNGTPLLQTIVNALNSNTTARELPDISPDVKILFLAGHDTNIANIGGMLGMTWTLPGQPDNTPPGGALLFELWSDKAGTQYVSVHMVYQTLAQLRNMTPLTLKEPAGTVPLKIPGCDDQTAEGYCPLDTFTRLVNQVVEPACQI

SEQ ID NO: 22 - protein, 413 aa, synthetic construct
  REGION 1..413: note = PHY-13789
SEAAPAGYHLQKAVILSRHGVRAPTKMTQLMRDVTPYTWPEWPVPLGYITPRGEHLVKLMGGFYRQYFQQQGLLSRDNCPTPDDVYVWTDVNQRTRKTGEAFLAGLAPECDLTIHHQSDIKQVDPLFHPLKAGICSMDKTQVQQAVEKQAGMPIDKLNQHYRAELALMSNVLNFPKSPYCQRHSGEQPCDFAQMFPSYLNISDDGNEVQLEGAVGLSSTLAEIFLLEYAQGMPVVAWGNIHSEQEWNDLLKLHNAYFDLMERTPYIAKHNGTPLLQTIVNALNPNATESKLPDISPDVKILFLAGHDTNIANIGGMLGMDWTLPGQPDNTPPGGALLFELWSDKEGTQYVSVKMVYQTLAQLRNMTPLTLKEPAGSVPLKIPGCDDQTAEGYCPLDTFTRLVNQVVEPACQLP

SEQ ID NO: 23 - protein, 413 aa, synthetic construct
  REGION 1..413: note = PHY-13798
SEAAPAGYHLQKAVILSRHGVRAPTKMTQTMRDVTPYTWPEWPVPLGYITPRGEHLVKLMGGFYRQYFQQQGLLSKDNCPTANDVYVWTDVAQRTRKTGEAFLAGLAPECDLTIHHQNNISQVDPLFHPVKAGICSMDKTQVQQAVEKQAGMPIDKLNQHYRPELALMSNVLNFPKSPYCQRHSGEKPCDFAQMMPSKLNISDDGNEVQLEGAVGLSSTLAEIFLLEYAQGMPVVAWGNIHSEQEWNDLLKLHNAHFDLMERTPYIAKHNGTPLLQAISNALNPNTTESKLPDISPDVKILFLAGHDTNIANIGGMLGMTWTLPGQPDNTPPGGALLFELWRDKAGTQYVSVHMVYQTLAQLRNMTPLTLKEPAGSVPLKIPGCDDQTAEGYCPLDTFTRLVNQVVEPACQLP

SEQ ID NO: 24 - protein, 413 aa, synthetic construct
  REGION 1..413: note = PHY-13868
SDTAPAGFQLEKVVILSRHGVRAPTKMTQLMRDVTPYTWPEWPVPLGYITPRGEHLISLMGGFYRQYFQQQGLLSKDNCPTANDVYVWTDVDQRTRKTGEAFLAGLAPECDLTIHHQSDIKQADPLFHPVKAGICSMDKSQTYEAVEKQAGGPIETLNQHYRPELALMSNVLNFPKSPYCRRHSGDKTCDFANAFPSYLNISDDGNEVQLEGAVGLSSTLAEIFLLEYAQGMPVVAWGNIHSEQEWNDLLKLHNAHFDLMQRTPYIAKHQGTPLLQTIVNALNSNTTARELPDISPDVKILFLAGHDTNIANIGGMLGMTWTLPGQPDNTPPGGALLFELWSDKEGTQYVSVEMVYQTLAQLRNMTPLTLKEPAGTVPLKIPGCDDQTAEGYCPLDTFTRLVNQVVEPACQLP

SEQ ID NO: 25 - protein, 413 aa, synthetic construct
  REGION 1..413: note = PHY-13883
SEAAPSGYQLEKVVILSRHGVRAPTKMTQTMRDVTPYTWPEWPVPLGYITPRGEHLVKLMGGFYRQYFQQQGLLSRDNCPTANDVYVWTDVDQRTRKTGEAFLAGLAPGCDLTIHHQNNISQADPLFHPVKAGICSMDKTQVQQAVEKQAGMPIDKLNQHYRAELALMSNVLNFPKSPYCRQHSVEQPCDFANAFPSYLNISDDGNEVQLEGAVGLSSTLAEIFLLEYAQGMPVVAWGNIHSEQEWNALLKLHNAHFDLMQRTPYIAKHNGTPLLQTIVNALNSNTTARELPDISPDVKILFLAGHDTNIANIGGMLGMTWTLPGQPDNTPPGGALLFELWRDKDGTQYVSVHMVYQTLAQLRNMTPLTLKEPAGSVPLKIPGCDDQTAEGYCPLDTFTRLVNQVVEPACQLP

SEQ ID NO: 26 - protein, 413 aa, synthetic construct
  REGION 1..413: note = PHY-13885
SEAAPSGYHLDKAVILSRHGVRAPTKMTQLMRDVTPYTWPEWPVPLGYITPRGEHLVSLMGGFYRQYFQQQGLLSRDRCPTANDVYVWTDVNQRTRKTGEAFLAGLAPECDLTIHHQNDIKQVDPLFHPLKAGICSMDKTQVQQAVEKQAGMPIDKLNQHYRPELALMSNVLNFPKSPYCRQHSVEQPCDFANAFPSYLNISDDGNEVQLEGAVGLSSTLAEIFLLEYAQGMPVVAWGNIHSEQEWNDLLKLHNAQFDLMERTPYIAKHQGTPLLQTIVNALNSNTTESKLPDISPSVKILFLAGHDTNIANIGGMLGMTWTLPGQPDNTPPGGALLFELWSDKDGTQYVSVKMVYQTLAQLRNMTPLTLKEPAGSVPLKIPGCDDQTAEGYCPLDTFTRLVNQVVEPACQLP

SEQ ID NO: 27 - protein, 413 aa, synthetic construct
  REGION 1..413: note = PHY-13936
SEAAPAGYHLDKAVILSRHGVRAPTKMTQTMRDVTPYTWPEWPVPLGYITPRGEHLVKLMGGFYRQYFQQQGLLPRDSCPTANDVYVWTDVNQRTRKTGEAFLAGLAPECAITIHHQSDIKQVDPLFHPVKAGICSMDKTQVQQAVEKQAGMPIDKLNQHYRPELALMSNVLNFPKSPYCQRHSGEQPCDFANAFPSYLNISDDGNEVQLEGAVGLSSTLAEIFLLEYAQGMPVVAWGNIHSEQEWNALLKLHNAHFDLMERTPYIAKHNGTPLLQTIVNALNSNTTESKLPDISPSVKILFLAGHDTNIANIGGMLGMTWTLPGQPDNTPPGGALLFELWSDKEGTQYVSVEMVYQTLAQLRNQTPLTLKEPAGSVPLKIPGCDDQTAEGYCPLDTFTRLVNQVVEPACQLP

SEQ ID NO: 28 - protein, 412 aa, synthetic construct
  REGION 1..412: note = PHY-14004
AEEANGMKLQKAVILSRHGVRAPTKMTQLMRDVTPYTWPEWPVPLGYITPRGEHLVSLMGGFYRQYFQQQGLLSRDSCPTANDVYVWTDVNQRTRKTGEAFLAGLAPGCDLTIHHQNDIKQVDPLFHPLKAGICSMDKTQVQQAVEKQAGMPIDKLNQHYRPELALMSNVLNFPKSPYCQRHSGEQPCDFANMFPSYLNISDDGNEVQLEGAVGLSSTLAEIFLLEYAQGMPVVAWGNIHSEQEWNDLLKLHNAQFDLMERTPYIAKHNGTPLLQTIVNALNSNTTARELPDISPDVKILFLAGHDTNIANIAGMLGMTWTLPGQPDNTPPGGALLFELWSDKDGTQYVSVHMVYQTLAQLRNQTPLTLKEPAGSVPLKIPGCDDQTAEGYCPLDTFTRLVNQVVEPACQLP

SEQ ID NO: 29 - protein, 413 aa, synthetic construct
  REGION 1..413: note = PHY-14215
SEAAPAGYHLQKAVILSRHGVRAPTKMTQLMRDVTPYTWPEWPVPLGYITPRGEHLVSLMGGFYRQYFQQQGLLSRDSCPTANDVYVWTDVDQRTRKTGEAFLAGLAPGCDLAIHHQQNITQVDPLFHPLKAGICSMDKTQVQQAVEKQAGMPIDKLNQHYRPELALMSNVLNFPKSPYCRRHSGDKTCDFANAFPSYLNISDDGNKVALDGAVGLSSTLAEIFLLEYAQGMPVVAWGNIHSESQWKSLLNLHNAHFNLMQRTPYIAKHNGTPLLQTIVNALNPNATESKLPDISPDNKILFLAGHDTNIANIGGMLGMTWTLPGQPDNTPPGGALLFELWRDKEGTQYVSVEMVYQTLAQLRNQTPLTLKEPAGSVPLKIPGCDDQTAEGYCPLDTFTRLVNQVVEPACQLP

SEQ ID NO: 30 - protein, 413 aa, synthetic construct
  REGION 1..413: note = PHY-14256
SDTAPAGYQLDKAVILSRHGVRAPTKMTQLMRDVTPYTWPEWPVPLGYITPRGEHLVKLMGGFYRQYFQQQGLLSKDNCPTANDVYVWTDVNQRTRKTGEAFLAGLAPGCDLTIHHQNDIKQADPLFHPLKAGICSMDKTQVQQAVEKQAGMPIDKLNQHYRPELALMSNVLNFPKSPYCQRHSVDKPCDFANAFPSYLNISDDGNEVQLEGAVGLSSTLAEIFLLEYAQGMPVVAWGNIHSEQEWNDLLKLHNAYFDLMQRTPYIAKHQGTPLLQTIVNALNSNTTESKLPDISPDVKILFLAGHDTNIANIAGMLGMDWTLPGQPDNTPPGGALLFELWSDKEGTQYVSVKMVYQTLAQLRNQTPLTLKEPAGSVPLKIPGCDDQTAEGYCPLDTFTRLVNQVVEPACQLP

SEQ ID NO: 31 - protein, 412 aa, synthetic construct
  REGION 1..412: note = PHY-14277
SEAAPAGYHLDKAVILSRHGVRAPTKMTQLMRDVTPYTWPEWPVPLGYITPRGEHLVSLMGGFYRQYFQQQGLLPKDNCPTANDVYVWTDVNQRTRKTGEAFLAGLAPECDLTIHHQNDIKQVDPLFHPVKAGICSMDKTQVQQAVEKQAGMPIDKLNQHYRPELALMSNVLNFPKSPYCRRHSGDKTCDFANAFPSYLNISDDGNEVQLEGAVGLSSTLAEIFLLEYAQGMPVVAWGNIHSEQEWNDLLKLHNAQFDLMERTPYIAKHQGTPLLQTIVNALNSNTTESKLPDISPDVKILFLAGHDTNIANIGGMLGMTWTLPGQPDNTPPGGALLFELWSDKEGTQYVSVKMVYQTLAQLRNMTPLTLKEPAGTVPLKIPGCDDQTAEGYCPLDTFTRLVNQVVEPACQI

SEQ ID NO: 32 - protein, 412 aa, synthetic construct
  REGION 1..412: note = PHY-14473
AEEQNGMKLQKAVILSRHGVRAPTKMTQLMRDVTPYTWPEWPVPLGYITPRGEHLVSLMGGFYRQYFQQQGLLSRDRCPTANDVYVWTDVNQRTRKTGEAFLAGLAPECAITIHHQSDIKQVDPLFHPVKAGICSMDKTQVQQAVEKQAGMPIDKLNQHYRPELALMSNVLNFPKSPYCRHQSGDKTCDFANAFPSYLNISDDGNEVQLEGAVGLSSTLAEIFLLEYAQGMPVVAWGNIHSEQEWNDLLKLHNAQFDLMERTPYIAKHNGTPLLQTIVNALNSNTTESKLPDISPDVKILFLAGHDTNIANIGGMLGMTWTLPGQPDNTPPGGALLFELWSDKEGTQYVSVKMVYQTLAQLRNQTPLTLKEPAGSVPLKIPGCDDQTAEGYCPLDTFTRLVNQVVEPACQLP

SEQ ID NO: 33 - protein, 413 aa, synthetic construct
  REGION 1..413: note = PHY-14614
SEAAPSGYHLDKAVILSRHGVRAPTKMTQTMRDVTPYTWPEWPVPLGYITPRGEHLVSLMGGFYRQYFQQQGLLSRDSCPTANDVYVWTDVNQRTRKTGEAFLAGLAPGCDLTIHHQQNITQVDPLFHPVKAGICSMDKTQVQQAVEKQAGMPIDKLNQHYRPELALMSNVLNFPKSPYCQQHSVDKPCDFANMFPSYLNISDDGNEVQLEGAVGLSSTLAEIFLLEYAQGMPVVAWGNIHSEQEWNDLLKLHNAQFDLMERTPYIAKHNGTPLLQTIVNALNSNTTESKLPDISPDVKILFLAGHDTNIANIAGMLGMDWTLPGQPDNTPPGGALLFELWSDKEGTQYVSVKMVYQTLAQLRNMTPLTLKEPAGTVPLKIPGCDDQTAEGYCPLDTFTRLVNQVVEPACQLP

SEQ ID NO: 34 - protein, 413 aa, synthetic construct
  REGION 1..413: note = PHY-14804
SDTAPAGFQLDKAVILSRHGVRAPTKMTQTMRDVTPYTWPEWPVPLGYITPRGEHLVSLMGGFYRQYFQQQGLLPRDNCPTANDVYVWTDVDQRTRKTGEAFLAGLAPECDLTIHHQSDIKQVDPLFHPLKAGICSMDKTQVQQAVEKQAGMPIDELNQHYRAELALMSNVLNFPKSPYCQRHSGEQPCDFANAFPSYLNISDDGNEVQLEGAVGLSSTLAEIFLLEYAQGMPVVAWGNIHSEQEWNDLLKLHNAQFDLMERTPYIAKHNGTPLLQTIVNALNSNTTARELPDISPDVKILFLAGHDTNIANIGGMLGMTWTLPGQPDNTPPGGALLFELWSDKDGTQYVSVKMVYQTLAQLRNQTPLTLKEPAGSVPLKIPGCDDQTAEGYCPLDTFTRLVNQVVEPACQLP

SEQ ID NO: 35 - protein, 413 aa, synthetic construct
  REGION 1..413: note = PHY-14945
SEAAPAGYHLDKAVILSRHGVRAPTKMTQTMRDVTPYTWPEWPVPLGYITPRGEHLVKLMGGFYRERFQQQGLLSRDSCPTANDVYVWTDVNQRTRKTGEAFLAGLAPECAITIHHQSDIKQVDPLFHPVKAGICSMDKTQVQQAVEKQAGMPIDKLNQHYRPELALMSNVLNFPKSPYCQRHSGEQPCDFANAFPSKLNISDDGNEVQLEGAVGLSSTLAEIFLLEYAQGMPVVAWGNIHSEQEWNDLLKLHNAHFDLMERTPYIAKHNGTPLLQTIVNALNSNTTARELPDISPDVKILFLAGHDTNIANIGGMLGMTWTLPGQPDNTPPGGALLFELWSDKEGTQYVSVKMVYQTLAQLRNQTPLTLKEPAGSVPLKIPGCDDQTAEGYCPLDTFTRLVNQVVEPACQLP

SEQ ID NO: 36 - protein, 412 aa, synthetic construct
  REGION 1..412: note = PHY-15459
AEPQNGMKLDKAVILSRHGVRAPTKMTQTMRDVTPYTWPEWPVPLGYITPRGEHLVSLMGGFYRQYFQQQGILSKDRCPRANDVYVWTDVDQRTRKTGEAFLAGLAPQCPLTIHHQNDIKQVDPMFHPVKGGICSMDKTQVQQAVEKQAGMPIDKLNDHYRPELALMSAVLNFPASPYCQRHSGEQPCDFAQMMPSKLYITDDGNEVQLEGAVGLSSTLAEIFLLEYAQGMPVVAWGNIHSEQEWNALLKLHNAYFDLMYRTPYIAKHNGTPLLQTIVNALNSNTTARELPDISPDNRILFLAGHDTNIANIGGMLGMDWTLPGQPDNTPPGGALLFELWSDKEGTQYVSVHMVYQTLAQLRNQTPLTLQEPAGTVPLKIPSCDDQTAEGYCPLDTFTKLVNQVVEPACQIP

SEQ ID NO: 37 - protein, 413 aa, synthetic construct
  REGION 1..413: note = PHY-16513
SEAAPSGYHLDKAVILSRHGVRAPTKMTQTMRDVTPYTWPEWPVPLGYITPRGEHLVSLMGGFYRQYFQMQGLLSRDRCPNANDVYVWTDVDQRTRKTGEAFLRGLAPECDLTIHHQNDIKQVDPMFHPVKGGICSMDKTQVQQAVEKQAGMPIDKLNDHYRPELALMSAVLNFPASPYCQQHSVDKPCDFANAFPSKLNITDDGNEVALDGAVGLSSTLAEIFLLEYAQGMPVVAWGNIHNESQWKSLLNLHNAHFNLMHRTPYIAKHNGTPLLQTIVNALNSNTTARELPDISPSVKILFLAGHDTNIANIGGMLGMDWTLPGQPDNTPPGGALLFELWSDKDGTQYVSVKMVYQTLAQLRNQTPLTLNEPAGTVPLKIPSCDDQTAEGYCPLDTFTVLVNQVVEPACQIP

(19) United States
(12) Patent Application Publication
(10) Pub. No.: US 2026/0355292 A1
(43) Pub. Date: Nov. 12, 2026

(54) FEED FORMULATIONS COMPRISING A PHYTASE FOR DAIRY RUMINANT ANIMALS

(71) Applicant: INTERNATIONAL N&H DENMARK APS, Kongens Lyyngby (DK)

(72) Inventors: Leon MARCHAL, Oegstgeest (NL); Yueming DERSJANT-LI, Wiltshire (UA); Trine CHRISTENSEN, Risskov (DK); Rubén GARCIA-GONZÁLEZ, Barcelona (ES); Alessandro MEREU, Barcelona (ES)

(21) Appl. No.: 19/133,298

(22) PCT Filed: Dec. 7, 2023
(86) PCT No.: PCT/US2023/082915

§ 371 Date: May 28, 2025

Related U.S. Application Data
(60) Provisional application No. 63/386,738, filed on Dec. 9, 2022.

(51) Int. Cl. A61K 38/46; A23K 10/38; A23K 20/189; A23K 50/10

(52) U.S. Cl. CPC A61K 38/465; A23K 10/38; A23K 20/189; A23K 50/10

(57) ABSTRACT

Provided herein are diets for dairy ruminant animals containing phytase polypeptides or fragments thereof wherein the diet contains decreased or no added inorganic phosphate.

### CROSS-REFERENCE TO RELATED APPLICATIONS

This application claims priority to U.S. Provisional Patent Application No. 63/386,738, filed Dec. 9, 2022, the content of which is hereby incorporated by reference herein in its entirety.

### INCORPORATION OF A SEQUENCE LISTING BY REFERENCE

The sequence listing provided in the file named 20231127_NB42148-WO-PCT_Sequence Listing.xml with a size of 61,709 bytes which was created on Nov. 27, 2023 and which is filed herewith, is incorporated by reference herein in its entirety.

### FIELD

The field pertains to ruminant diets containing no or substantially no or decreased inorganic phosphate as well as engineered phytase polypeptides and uses of the same for enhancing dairy production in ruminant animals.

### BACKGROUND

Ruminant livestock accounts for up to 70% of the phosphorus (P) waste produced by farmed animals (Tamminga and Verstegen, 1992). Reducing P excretion from dairy cattle continues to be a major focus to minimize the negative impacts of excess P on the environment, especially on aquatic ecosystems (Sharpley et al., 1994; NRC, 2001). Optimizing dietary P content and improving P utilization are key strategies for reducing P excretion in dairy cows (Valk et al., 2000). The latter could enable a reduction in the P content of the diet so that dietary levels are fed closer to animal requirements, reducing feed costs. It could also increase the sustainability of the diet, allowing more flexibility in the choice of P-containing ingredients and reducing reliance on inorganic phosphates (Pi) that are readily absorbed but are costly and originate from a finite resource.

Traditionally, it has been believed that phytase supplementation in dairy cattle diets is not warranted. This is due to the substantial phytase activity of ruminal bacteria that hydrolyze phytate (myo-inositol hexakisphosphate, IP6), releasing Pi for later absorption in the small intestine (Raun et al., 1956; Yanke et al., 1998; Guyton et al., 2003). The seminal study of Morse ct al. (1992) reported that phytate-P (PP) disappearance from a range of cercal and oilseed meal concentrates incubated with ruminal fluid was greater than 99% in vitro, and that apparent total tract digestibility (ATTD) of PP was 94 to 99% in lactating dairy cows. Similarly, Clark et al. (1986) reported ATTD of PP values higher than 95% in high producing dairy cows. However, more recent studies have reported lower and more variable estimates of ATTD of PP, of between 69 and 97% (Kinkaid et al. 2005; Brask-Pedersen et al. 2013; Jarrett et al. 2014).

In practice, phytate degradability in the rumen may vary in relation to a variety of factors. These include the composition of the diet and forage-to-concentrate ratio that may alter the ruminal microflora and hence their capacity to digest phytate (Yanke et al., 1998; Humer and Zebeli, 2015), the ruminal P availability of individual ingredients (Taylor et al., 2001; Haese et al., 2020) that may be influenced by processing methods designed to protect proteins from ruminal degradation (Bravo et al., 2000), and the faster flow rate of the digesta of modern high producing cows fed easily fermentable carbohydrate that may result in some PP leaving the rumen undegraded (Krämer et al., 2013; Humer and Zebeli, 2015). Furthermore, the neutral pH of the rumen that is typically pH 6.0 to 7.0 (Winter et al., 2015; Kim et al., 2018) may not be optimal for ruminal microbes producing cysteine phytases that exhibit optimum activity at pH 4.5 (Yanke et al., 1998; Puhl et al., 2008).

Exogenous microbial phytases, mostly of bacterial origin, are widely used as feed additives in commercial poultry and swine diets for improving P digestibility and utilization via their hydrolysis of phytate (Selle and Ravindran, 2007; Humer et al., 2015). Beneficial effects of exogenous phytase on the digestion and utilization of other nutrients including protein, amino acids (AA), energy and starch are also evident in monogastric animals, especially in poultry (Selle et al., 2000; Ravindran et al., 2006; Truong et al., 2014; Truong et al., 2015). Literature on the effects of exogenous phytase on nutrient digestibility in lactating dairy cows is more limited but some studies have evaluated the potential for phytase to improve PP digestibility (Kincaid et al., 2005; Brask-Pedersen et al., 2013; Winter et al., 2015; Giagnoni et al., 2021; see also International Patent Application Publication No. WO2021127360, each of which is incorporated by reference herein) to limited degrees of success.

Accordingly, a need exists for the development of diets for lactating ruminant animals that are substantially or completely inorganic phosphate free, while still ensuring normal or improved protein utilization during lactation compared to exogenous phytase-free diets that are supplemented with sources of inorganic phosphate.

### SUMMARY

Provided herein, inter alia, are ruminant animal diets containing phytases which are free or substantially free of exogenously added inorganic phosphate or contain substantially reduced exogenously added inorganic phosphate. When fed to ruminant animals (for example, lactating ruminant animals), these diets ensure improved phosphorous and/or protein utilization (manifested by, for example, improved digestibility, decreased excretion of crude protein, and/improved milk protein content and/or yield) compared to diets which contain inorganic phosphate supplementation.

Accordingly, in one aspect, provided herein are methods for improving phosphorous and/or protein utilization in a lactating ruminant animal comprising administering to the animal a phytase polypeptide or a fragment thereof comprising phytase activity. In some embodiments, the phytase polypeptide or a fragment thereof comprises at least 82% sequence identity with the amino acid sequence set forth in SEQ ID NO:1. In some embodiments of any of the embodiments disclosed herein, improving protein utilization comprises a) improved digestibility and/or decreased excretion of crude protein; and/or b) improved milk protein content and protein yield. In some embodiments of any of the embodiments disclosed herein, improved phosphorous utilization comprises improved digestibility and/or decreased excretion of total phosphorus and/or phytate bound phosphorous. In some embodiments of any of the embodiments disclosed herein, the method further improves calcium digestibility. In some embodiments of any of the embodiments disclosed herein, the lactating ruminant animal is selected from the group consisting of cattle, young calves, goats, sheep, giraffes, bison, moose, elk, yaks, water buffalo, deer, reindeer, caribou, camels, alpacas, llamas, antelope, pronghorn and nilgai. In some embodiments of any of the embodiments disclosed herein, the phytase polypeptide or a fragment thereof comprising phytase activity is not coated. In some embodiments of any of the embodiments disclosed herein, the phytase polypeptide or a fragment thereof comprising phytase activity is formulated for bypassing the rumen. In some embodiments of any of the embodiments disclosed herein, the phytase polypeptide or a fragment thereof comprising phytase activity is administered in combination with a feed. In some embodiments, the feed is selected from the group consisting of total mixed ration (TMR), compound feed, mineral premix. In some embodiments, the mineral premix is administered as a licking block. In some embodiments of any of the embodiments disclosed herein, the phytase polypeptide or a fragment thereof comprising phytase activity is administered in a water line. In some embodiments of any of the embodiments disclosed herein, the method further comprises administering at least one additional enzyme to the animal. In some embodiments of any of the embodiments disclosed herein, the phytase polypeptide is administered in conjunction with a diet having low phosphorous content. In some embodiments of any of the embodiments disclosed herein, the phytase polypeptide or a fragment thereof comprises SEQ ID NO:1, SEQ ID NO:2, SEQ ID NO: 3, SEQ ID NO:4, SEQ ID NO:5, SEQ ID NO:6, SEQ ID NO:7, SEQ ID NO:8, SEQ ID NO: 9, SEQ ID NO: 10, SEQ ID NO:11, SEQ ID NO:12, SEQ ID NO: 13, SEQ ID NO: 14, SEQ ID NO: 15, SEQ ID NO:16, SEQ ID NO: 17, SEQ ID NO:18, SEQ ID NO: 19, SEQ ID NO:20, SEQ ID NO:21, SEQ ID NO:22, SEQ ID NO:23, SEQ ID NO:24, SEQ ID NO:25, SEQ ID NO: 26, SEQ ID NO:27, SEQ ID NO:28, SEQ ID NO:29, SEQ ID NO:30, SEQ ID NO:31, SEQ ID NO: 32, SEQ ID NO:33, SEQ ID NO:34, SEQ ID NO:35, SEQ ID NO:36, or SEQ ID NO:37.

In another aspect, provided herein is a method for decreasing phosphate and/or nitrogen excretion in the feces of a lactating ruminant animal comprising administering to the animal a phytase polypeptide or a fragment thereof comprising phytase activity. In some embodiments, the phytase polypeptide or a fragment thereof comprises at least 82% sequence identity with the amino acid sequence set forth in SEQ ID NO:1. In some embodiments of any of the embodiments disclosed herein, the lactating ruminant animal is selected from the group consisting of cattle, young calves, goats, sheep, giraffes, bison, moose, elk, yaks, water buffalo, deer, reindeer, caribou, camels, alpacas, llamas, antelope, pronghorn and nilgai. In some embodiments of any of the embodiments disclosed herein, the phytase polypeptide or a fragment thereof comprising phytase activity is not coated. In some embodiments of any of the embodiments disclosed herein, the phytase polypeptide or a fragment thereof comprising phytase activity is formulated for bypassing the rumen. In some embodiments of any of the embodiments disclosed herein, the phytase polypeptide or a fragment thereof comprising phytase activity is administered in combination with a feed. In some embodiments, the feed is selected from the group consisting of total mixed ration (TMR), compound feed, mineral premix. In some embodiments, the mineral premix is administered as a licking block. In some embodiments of any of the embodiments disclosed herein, the phytase polypeptide or a fragment thereof comprising phytase activity is administered in a water line. In some embodiments of any of the embodiments disclosed herein, the method further comprises administering at least one additional enzyme to the animal. In some embodiments of any of the embodiments disclosed herein, the phytase polypeptide is administered in conjunction with a diet having low phosphorous content. In some embodiments of any of the embodiments disclosed herein, the phytase polypeptide or a fragment thereof comprises SEQ ID NO:1, SEQ ID NO:2, SEQ ID NO: 3, SEQ ID NO:4, SEQ ID NO:5, SEQ ID NO:6, SEQ ID NO:7, SEQ ID NO:8, SEQ ID NO: 9, SEQ ID NO: 10, SEQ ID NO:11, SEQ ID NO: 12, SEQ ID NO:13, SEQ ID NO: 14, SEQ ID NO: 15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO: 26, SEQ ID NO:27, SEQ ID NO:28, SEQ ID NO:29, SEQ ID NO:30, SEQ ID NO:31, SEQ ID NO: 32, SEQ ID NO:33, SEQ ID NO:34, SEQ ID NO:35, SEQ ID NO:36, or SEQ ID NO:37.

In still further aspects, provided herein is a method for degrading phytate in the abomasum of a ruminant animal comprising administering to the animal a phytase polypeptide or a fragment thereof comprising at least 82% sequence identity of the amino acid sequence set forth in SEQ ID NO:1. In some embodiments, the phytase polypeptide or a fragment thereof degrades phytate in the abomasum to a greater extent as compared to a phytase polypeptide or a fragment thereof that does not comprise at least 82% sequence identity of the amino acid sequence set forth in SEQ ID NO:1. In some embodiments of any of the embodiments disclosed herein, the lactating ruminant animal is selected from the group consisting of cattle, young calves, goats, sheep, giraffes, bison, moose, elk, yaks, water buffalo, deer, reindeer, caribou, camels, alpacas, llamas, antelope, pronghorn and nilgai. In some embodiments of any of the embodiments disclosed herein, the phytase polypeptide or a fragment thereof comprising phytase activity is not coated. In some embodiments of any of the embodiments disclosed herein, the phytase polypeptide or a fragment thereof comprising phytase activity is administered in combination with a feed. In some embodiments, the feed is selected from the group consisting of total mixed ration (TMR), compound feed, mineral premix. In some embodiments, the feed comprises one or more cereal byproducts of a distillation process. In some embodiments, the phytase polypeptide or a fragment thereof comprising phytase activity is used during a saccharification and/or fermentation reaction prior to the distillation process. In some embodiments of any of the embodiments disclosed herein, the feed comprises one or more of corn gluten meal, Distillers Dried Grains with Solubles (DDGS), corn based Distillers Dried Grains with Solubles (cDDGS), wheat bran, wheat middlings, wheat shorts, rice bran, rice hulls, oat hulls, palm kernel, or citrus pulp. In some embodiments, the mineral premix is administered as a licking block. In some embodiments of any of the embodiments disclosed herein, the phytase polypeptide or a fragment thereof comprising phytase activity is administered in a water line. In some embodiments of any of the embodiments disclosed herein, the method further comprises administering at least one additional enzyme to the animal. In some embodiments of any of the embodiments disclosed herein, the phytase polypeptide is administered in conjunction with a diet having low phosphorous content. In some embodiments of any of the embodiments disclosed herein, the phytase polypeptide or a fragment thereof comprises SEQ ID NO:1, SEQ ID NO: 2, SEQ ID NO:3, SEQ ID NO:4, SEQ ID NO:5, SEQ ID NO:6, SEQ ID NO:7, SEQ ID NO: 8, SEQ ID NO:9, SEQ ID NO:10, SEQ ID NO:11, SEQ ID NO: 12, SEQ ID NO:13, SEQ ID NO: 14, SEQ ID NO:15, SEQ ID NO: 16, SEQ ID NO: 17, SEQ ID NO: 18, SEQ ID NO: 19, SEQ ID NO: 20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO: 31, SEQ ID NO:32, SEQ ID NO:33, SEQ ID NO:34, SEQ ID NO:35, SEQ ID NO:36, or SEQ ID NO:37.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIG. 1 is a graph depicting the in vitro evaluation showing percentage (%) of undegraded IP6 during rumen (pH 6.2) and abomasum (pH 2.0) simulation. Three phytase treatments were evaluated: PhyG, Ronozyme® HiPhos and Natuphos® E along with control (no phytase added).

### DETAILED DESCRIPTION

There is a growing global sustainability awareness regarding the proper use of finite resources like inorganic phosphorus (P) and reduction of P pollution. Cattle have among the highest environmental impact of all animal proteins, using significant amounts of inorganic phosphate. The inventors of the present application have surprisingly discovered that use of next generation biosynthetic bacterial 6-phytases in lactating ruminant diets can decrease or eliminate the need to supplement the diet with one or more sources of inorganic phosphate and/or can substantially reduce the need to supplement the diet with one or more sources of inorganic phosphate. Consequently, use of the phytase supplemented inorganic phosphate-free diets disclosed herein provides both an economic advantage in the form of decreased costs of feed as well as a significant environmental benefit due to decreased phosphate pollution as a byproduct of large-scale dairy production.

All patents, patent applications, and publications cited are incorporated herein by reference in their entirety.

In this disclosure, many terms and abbreviations are used. The following definitions apply unless specifically stated otherwise.

As used herein, the singular forms “a,” “an,” and “the” include plural references unless the context clearly dictates otherwise. For example, the term “a compound” or “at least one compound” may include a plurality of compounds, including mixtures thereof. The terms “a,” “an,” “the,” “one or more,” and “at least one,” for example, can be used interchangeably herein.

The term “and/or” and “or” are used interchangeably herein and refer to a specific disclosure of each of the two specified features or components with or without the other. Thus, the term “and/or” as used in a phrase such “A and/or B” herein is intended to include “A and B,” “A or B,” “A” (alone), and “B” alone. Likewise, the term “and/or” as used a phrase such as “A, B and/or C” is intended to encompass each of the following aspects: A, B and C; A, B or C; A or C; A or B; B or C; A and C; A and B; B and C; A (alone); B (alone); and C (alone).

Words using the singular include the plural, and vice versa.

The terms “comprises,” “comprising,” “includes,” “including,” “having” and their conjugates are used interchangeably and mean “including but not limited to.” It is understood that wherever aspects are described herein with the language “comprising,” otherwise analogous aspects described in terms of “consisting of’ and/or “consisting essentially of “are also provided.

The term “consisting of” means “including and limited to.”

The term “consisting essentially of” means the specified material of a composition, or the specified steps of a methods, and those additional materials or steps that do not materially affect the basic characteristics of the material or method.

Throughout this application, various embodiments can be presented in a range format. It should be understood that the description in range format is merely for convenience and brevity and should not be construed as an inflexible limitation on the scope of the embodiments described herein. Accordingly, the description of a range should be considered to have specifically disclosed all the possible subranges as well as individual numerical values within that range. For example, description of a range, such as from 1 to 6 should be considered to have specifically disclosed subranges such as from 1 to 2, from 1 to 3, from 1 to 4 and from 1 to 5, from 2 to 3, from 2 to 4, from 2 to 5, from 2 to 6, from 3 to 4, from 3 to 5, from 3 to 6, etc. as well as individual numbers within that range, for example, 1, 2, 3, 4, 5 and 6. This applies regardless of the breadth of the range.

The term “about” as used herein can allow for a degree of variability in a value or range, for example, within 10%, within 5%, or within 1% of a stated value or of a stated limit of a range.

The term “phytase” (myo-inositol hexakisphosphate phosphohydrolase) refers to a class of phosphatase enzymes that catalyzes the hydrolysis of phytic acid (myo-inositol hexakisphosphate or IP6)—an indigestible, organic form of phosphorus that is found in grains and oil seeds—and releases a usable form of inorganic phosphorus.

The term “ruminants” or “ruminant animals” as used herein refers to mammals, both males and females, that are able to acquire nutrients from plant-based food through fermentation in a specialized stomach chamber prior to digestion, principally through bacterial actions. The process typically requires regurgitation of fermented ingesta (known as cud) and chewing it again. The process of rechewing the cud to further break down plant matter and stimulate digestion is called “rumination”. The primary difference between ruminant animals and non-ruminant animals is that ruminant animals have a three or four-chambered stomach. The group includes, among others, deer, antelopes, buffalo, cattle, sheep, camel, and goat.

The term “lactating ruminant” as used herein refers to a ruminant animal which is capable of producing milk post-parturition.

The term “dairy ruminant” as used herein refers to a ruminant animal, whose milk is used for commercial purposes including the period that the animal is not giving milk.

The terms “feed,” an “animal feed,” or “diet” are used interchangeably herein to mean any natural or artificial diet, meal or the like or components of such meals intended or suitable for being eaten, taken in, digested, by a non-human animal, respectively. Preferably term “feed” is used with reference to products that are fed to animals in the rearing of livestock.

A “feed additive” as used herein refers to one or more ingredients, products of substances (e.g., cells), used alone or together, in nutrition (e.g., to improve the quality of a food (e.g., an animal feed), to improve an animal's performance and/or health, and/or to enhance digestibility of a food or materials within a food.

As used herein, the term “food” is used in a broad sense- and covers food and food products in any form for humans as well as food for animals (i.e. a feed).

The food or feed may be in the form of a solution or as a solid-depending on the use and/or the mode of application and/or the mode of administration. In some embodiments, the enzymes mentioned herein may be used as—or in the preparation or production of—a food or feed substance.

As used herein the term “food or feed ingredient” includes a formulation, which is or can be added to foods or foodstuffs and includes formulations which can be used at low levels in a wide variety of products. The food ingredient may be in the form of a solution or as a solid-depending on the use and/or the mode of application and/or the mode of administration. The enzymes described herein may be used as a food or feed ingredient or in the preparation or production. The enzymes may be—or may not be added to—food supplements. Feed compositions for monogastric animals typically include compositions comprising plant products which contain phytate. Such compositions include, but are not limited to, cornmeal, soybean meal, rapeseed meal, sunflower meal, cottonseed meal, maize, wheat, barley and sorghum-based feeds.

As used herein, the term “pelleting” refers to the production of pellets which can be solid, rounded, spherical and cylindrical tablets, particularly feed pellets and solid, extruded animal feed. One example of a known feed pelleting manufacturing process generally includes admixing together food or feed ingredients at least 1 minutes at room temperature, transferring the admixture to a surge bin, conveying the admixture to a steam conditioner (i.e., conditioning), optionally transferring the steam conditioned admixture to an expander, transferring the admixture to the pellet mill or extruder, and finally transferring the pellets into a pellet cooler. (Fairfield, D. 1994. Chapter 10, Pelleting Cost Center. In Feed Manufacturing Technology IV. (McEllhiney, editor), American Feed Industry Association, Arlington, Va., pp. 110-139.).

The term “pellet” refers to a composition of animal feed (usually derived from grain) that has been subjected to a heat treatment, such as a steam treatment (i.e., conditioning), and pressed or extruded through a machine. The pellet may incorporate enzyme in the form of a liquid preparation or a dry preparation. The dry preparation may be coated or not coated and may be in the form of a granule. The term “granule” is used for particles composed of enzymes (such as a phytase, for example, any of the engineered phytase polypeptides disclosed herein) and other chemicals such as salts and sugars and may be formed using any of a variety of techniques, including fluid bed granulation approaches to form layered granules.

The term “specific activity” as used herein is the number of enzyme units per ml divided by the concentration of (total) protein in mg/ml. Specific activity values are therefore usually quoted as units/mg. Alternatively, specific activity is the number of enzyme units per ml divided by the concentration of phytase in mg/ml.

The term “isolated” means a substance in a form or environment that does not occur in nature and does not reflect the extent to which an isolate has been purified but indicates isolation or separation from a native form or native environment. Non-limiting examples of isolated substances include (1) any non-naturally occurring substance, (2) any substance including, but not limited to, any host cell, enzyme, engineered enzyme, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated. The terms “isolated nucleic acid molecule”, “isolated polynucleotide”, and “isolated nucleic acid fragment” will be used interchangeably and refer to a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

The terms “purify,” “purified,” and purification mean to make substantially pure or clear from unwanted components, material defilement, admixture or imperfection. For example, as applied to nucleic acids or polypeptides, purification generally denotes a nucleic acid or polypeptide that is essentially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

The terms “peptides”, “proteins” and “polypeptides are used interchangeably herein and refer to a polymer of amino acids joined together by peptide bonds. A “protein” or “polypeptide” comprises a polymeric sequence of amino acid residues. The single and 3-letter code for amino acids as defined in conformity with the IUPAC-IUB Joint Commission on Biochemical Nomenclature (JCBN) is used throughout this disclosure. The single letter X refers to any of the twenty amino acids. It is also understood that a polypeptide may be coded for by more than one nucleotide sequence due to the degeneracy of the genetic code. Mutations can be named by the one letter code for the parent amino acid, followed by a position number and then the one letter code for the variant amino acid. For example, mutating glycine (G) at position 87 to serine(S) is represented as “G087S” or “G87S”. When describing modifications, a position followed by amino acids listed in parentheses indicates a list of substitutions at that position by any of the listed amino acids. For example, 6 (L, I) means position 6 can be substituted with a leucine or isoleucine. At times, in a sequence, a slash (/) is used to define substitutions, e.g. F/V, indicates that the position may have a phenylalanine or valine at that position.

As used herein with regard to amino acid residue positions, “corresponding to” or “corresponds to” or “correspond to” or “corresponds” refers to an amino acid residue at the enumerated position in a protein or peptide, or an amino acid residue that is analogous, homologous, or equivalent to an enumerated residue in a protein or peptide. As used herein, “corresponding region” generally refers to an analogous position in a related protein or a reference protein.

The terms “derived from” and “obtained from” refer to not only a protein produced or producible by a strain of the organism in question, but also a protein encoded by a DNA sequence isolated from such strain and produced in a host organism containing such DNA sequence. Additionally, the term refers to a protein which is encoded by a DNA sequence of synthetic and/or cDNA origin and which has the identifying characteristics of the protein in question.

The term “amino acid” refers to the basic chemical structural unit of a protein or polypeptide. The following abbreviations used herein to identify specific amino acids can be found in Table A.

TABLE A
One and Three Letter Amino Acid Abbreviations

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Thermostable serine acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid or as defined herein | Xaa | X |

It would be recognized by one of ordinary skill in the art that modifications of amino acid sequences disclosed herein can be made while retaining the function associated with the disclosed amino acid sequences. For example, it is well known in the art that alterations in a gene which result in the production of a chemically equivalent amino acid at a given site, but do not affect the functional properties of the encoded protein are common.

The term “codon optimized”, as it refers to genes or coding regions of nucleic acid molecules for transformation of various hosts, refers to the alteration of codons in the gene or coding regions of the nucleic acid molecules to reflect the typical codon usage of the host organism without altering the polypeptide for which the DNA codes.

The term “transformation” as used herein refers to the transfer or introduction of a nucleic acid molecule into a host organism. The nucleic acid molecule may be introduced as a linear or circular form of DNA. The nucleic acid molecule may be a plasmid that replicates autonomously, or it may integrate into the genome of a production host. Production hosts containing the transformed nucleic acid are referred to as “transformed” or “recombinant” or “transgenic” organisms or “transformants”.

The terms “recombinant” and “engineered” refer to an artificial combination of two otherwise separated segments of nucleic acid sequences, e.g., by chemical synthesis or by the manipulation of isolated segments of nucleic acids by genetic engineering techniques. For example, DNA in which one or more segments or genes have been inserted, either naturally or by laboratory manipulation, from a different molecule, from another part of the same molecule, or an artificial sequence, resulting in the introduction of a new sequence in a gene and subsequently in an organism. The terms “recombinant”, “transgenic”, “transformed”, “engineered”, “genetically engineered” and “modified for exogenous gene expression” are used interchangeably herein.

The terms “recombinant construct”, “expression construct”, “recombinant expression construct” and “expression cassette” are used interchangeably herein. A recombinant construct comprises an artificial combination of nucleic acid fragments, e.g., regulatory and coding sequences that are not all found together in nature. For example, a construct may comprise regulatory sequences and coding sequences that are derived from different sources, or regulatory sequences and coding sequences derived from the same source but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells. The skilled artisan will also recognize that different independent transformation events may result in different levels and patterns of expression (Jones et al., (1985) EMBO J 4:2411-2418; De Almeida et al., (1989) Mol Gen Genetics 218:78-86), and thus that multiple events are typically screened to obtain lines displaying the desired expression level and pattern. Such screening may be accomplished using standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis.

The terms “production host”, “host” and “host cell” are used interchangeably herein and refer to any plant, organism, or cell of any plant or organism, whether human or non-human into which a recombinant construct can be stably or transiently introduced to express a gene. This term encompasses any progeny of a parent cell, which is not identical to the parent cell due to mutations that occur during propagation.

The term “percent identity” is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the number of matching nucleotides or amino acids between strings of such sequences. “Identity” and “similarity” can be readily calculated by known methods, including but not limited to those described in: Computational Molecular Biology (Lesk, A. M., cd.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Methods to determine identity and similarity are codified in publicly available computer programs.

As used herein, “% identity” or percent identity” or “PID” refers to protein sequence identity. Percent identity may be determined using standard techniques known in the art. Useful algorithms include the BLAST algorithms (See, Altschul et al., J Mol Biol, 215:403-410, 1990; and Karlin and Altschul, Proc Natl Acad Sci USA, 90:5873-5787, 1993). The BLAST program uses several search parameters, most of which are set to the default values. The NCBI BLAST algorithm finds the most relevant sequences in terms of biological similarity but is not recommended for query sequences of less than 20 residues (Altschul et al., Nucleic Acids Res, 25:3389-3402, 1997; and Schaffer et al., Nucleic Acids Res, 29:2994-3005, 2001). Exemplary default BLAST parameters for a nucleic acid sequence searches include: Neighboring words threshold=11; E-value cutoff=10; Scoring Matrix=NUC.3.1 (match=1, mismatch=−3); Gap Opening=5; and Gap Extension=2. Exemplary default BLAST parameters for amino acid sequence searches include: Word size=3; E-value cutoff=10; Scoring Matrix=BLOSUM62; Gap Opening=11; and Gap extension=1. A percent (%) amino acid sequence identity value is determined by the number of matching identical residues divided by the total number of residues of the “reference” sequence. BLAST algorithms refer to the “reference” sequence as the “query” sequence.

As used herein, “homologous proteins” or “homologous phytases” refers to proteins that have distinct similarity in primary, secondary, and/or tertiary structure. Protein homology can refer to the similarity in linear amino acid sequence when proteins are aligned. Homologous search of protein sequences can be done using BLASTP and PSI-BLAST from NCBI BLAST with threshold (E-value cut-off) at 0.001. (Altschul S F, Madde T L, Shaffer A A, Zhang J, Zhang Z, Miller W, Lipman D J. Gapped BLAST and PSI BLAST a new generation of protein database search programs. Nucleic Acids Res 1997 Set 1; 25 (17): 3389-402). Using this information, proteins sequences can be grouped.

Sequence alignments and percent identity calculations may be performed using the Megalign program of the LASERGENE bioinformatics computing suite (DNASTAR Inc., Madison, WI), the AlignX program of Vector NTI v. 7.0 (Informax, Inc., Bethesda, MD), or the EMBOSS Open Software Suite (EMBL-EBI; Rice et al., Trends in Genetics 16, (6): 276-277 (2000)). Multiple alignment of the sequences can be performed using the CLUSTAL method (such as CLUSTALW; for example, version 1.83) of alignment (Higgins and Sharp, CABIOS, 5:151-153 (1989); Higgins et al., Nucleic Acids Res. 22:4673-4680 (1994); and Chenna et al., Nucleic Acids Res 31 (13): 3497-500 (2003)), available from the European Molecular Biology Laboratory via the European Bioinformatics Institute) with the default parameters. Suitable parameters for CLUSTALW protein alignments include GAP Existence penalty=15, GAP extension=0.2, matrix=Gonnet (e.g., Gonnet250), protein ENDGAP=−1, protein GAPDIST=4, and KTUPLE=1. In one embodiment, a fast or slow alignment is used with the default settings where a slow alignment. Alternatively, the parameters using the CLUSTALW method (e.g., version 1.83) may be modified to also use KTUPLE=1, GAP PENALTY=10, GAP extension=1, matrix=BLOSUM (e.g., BLOSUM64), WINDOW=5, and TOP DIAGONALS SAVED=5. Alternatively, multiple sequence alignment may be derived using MAFFT alignment from Geneious® version 10.2.4 with default settings, scoring matrix BLOSUM62, gap open penalty 1.53 and offset value 0.123.

The MUSCLE program (Robert C. Edgar. MUSCLE: multiple sequence alignment with high accuracy and high throughput Nucl. Acids Res. (2004) 32 (5): 1792-1797) is yet another example of a multiple sequence alignment algorithm.

The term “engineered phytase polypeptide” means that the polypeptide is not naturally occurring and has phytase activity.

It is noted that a fragment of the engineered phytase polypeptide is a portion or subsequence of the engineered phytase polypeptide that is capable of functioning like the engineered phytase polypeptide, i.e., it retains phytase activity.

The term “vector” refers to a polynucleotide sequence designed to introduce nucleic acids into one or more cell types. Vectors include, but are not limited to, cloning vectors, expression vectors, shuttle vectors, plasmids, phage particles, cassettes and the like.

An “expression vector” as used herein means a DNA construct comprising a DNA sequence which is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “expression”, as used herein, refers to the production of a functional end-product (e.g., an mRNA or a protein) in either precursor or mature form. Expression may also refer to translation of mRNA into a polypeptide.

Expression of a gene involves transcription of the gene and translation of the mRNA into a precursor or mature protein. “Mature” protein refers to a post-translationally processed polypeptide; i.e., one from which any signal sequence, pre- or propeptides present in the primary translation product have been removed. “Precursor” protein refers to the primary product of translation of mRNA; i.e., with pre- and propeptides still present. Pre- and propeptides may be but are not limited to intracellular localization signals. “Stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance.

Thus, in one embodiment, there is described a recombinant construct comprising a regulatory sequence functional in a production host operably linked to a nucleotide sequence encoding an engineered phytase polypeptide and fragments thereof as described herein. This recombinant construct may comprise a regulatory sequence functional in a production host operably linked to a nucleotide sequence encoding any of the engineered phytase polypeptide and fragments thereof described herein. Furthermore, the production host is selected from the group consisting of bacteria, fungi, yeast, plants or algae. In some embodiments, the production host is the filamentous fungus, Trichoderma reesei.

Alternatively, it may be possible to use cell-free protein synthesis as described in Chong, Curr Protoc Mol Biol. 2014; 108:16.30.1-16.30.11.

Possible initiation control regions or promoters that can be included in the expression vector are numerous and familiar to those skilled in the art. A “constitutive promoter” is a promoter that is active under most environmental and developmental conditions. An “inducible” or “repressible” promoter is a promoter that is active under environmental or developmental regulation. In some embodiments, promoters are inducible or repressible due to changes in environmental factors including but not limited to, carbon, nitrogen or other nutrient availability, temperature, pH, osmolarity, the presence of heavy metal(s), the concentration of inhibitor(s), stress, or a combination of the foregoing, as is known in the art. In some embodiments, the inducible or repressible promoters are inducible or repressible by metabolic factors, such as the level of certain carbon sources, the level of certain energy sources, the level of certain catabolites, or a combination of the foregoing as is known in the art.

In one embodiment, the promoter is one that is native to the host cell. For example, in some instances when Trichoderma reesei is the host, the promoter can be a native T. reesei promoter such as the cbhl promoter which is deposited in GenBank under Accession Number D86235. Other suitable non-limiting examples of promoters useful for fungal expression include, cbh2, egl1, egl2, egl3, egl4, egl5, xyn1, and xyn2, repressible acid phosphatase gene (phoA) promoter of P. chrysogenus (see e.g., Graessle et al., (1997) Appl. Environ. Microbiol., 63:753-756), glucose repressible PCK1 promoter (see e.g., Leuker et al., (1997), Gene, 192:235-240), maltose inducible, glucose-repressible MET3 promoter (see Liu et al., (2006), Eukary. Cell, 5:638-649), pKi promoter and cpc1 promoter. Other examples of useful promoters include promoters from A. awamori and A. niger glucoamylase genes (see e.g., Nunberg et al., (1984) Mol. Cell Biol. 15 4:2306-2315 and Boel et al., (1984) EMBO J. 3:1581-1585). Also, the promoters of the T. reesei xlnl gene may be useful (see e.g., EPA 137280A1).

DNA fragments which control transcriptional termination may also be derived from various genes native to a preferred production host cell. In certain embodiments, the inclusion of a termination control region is optional. In certain embodiments, the expression vector includes a termination control region derived from the preferred host cell.

The terms “production host”, “production host cell”, “host cell” and “host strains” are used interchangeable herein and mean a suitable host for an expression vector or DNA construct comprising a polynucleotide encoding phytase polypeptide or fragment thereof. The choice of a production host can be selected from the group consisting of bacteria, fungi, yeast, plants and algae. Typically, the choice will depend upon the gene encoding the engineered phytase polypeptide or fragment thereof and its source.

Specifically, host strains are preferably filamentous fungal cells. In a preferred embodiment of the invention, “host cell” means both the cells and protoplasts created from the cells of a filamentous fungal strain and particularly a Trichoderma sp. or an Aspergillus sp.

The term “filamentous fungi” refers to all filamentous forms of the subdivision Eumycotina (See, Alexopoulos, C. J. (1962), INTRODUCTORY MYCOLOGY, Wiley, New York). These fungi are characterized by a vegetative mycelium with a cell wall composed of chitin, cellulose, and other complex polysaccharides. The filamentous fungi of the present invention are morphologically, physiologically, and genetically distinct from yeasts. Vegetative growth by filamentous fungi is by hyphal elongation and carbon catabolismis obligatory aerobic. In the present invention, the filamentous fungal parent cell may be a cell of a species of, but not limited to, Trichoderma, (e.g., Trichoderma reesei (previously classified as T. longibrachiatum and currently also known as Hypocrea jecorina), Trichoderma viride, Trichoderma koningii, Trichoderma harzianum); Penicillium sp., Humicola sp. (e.g., Humicola insolens and Humicola grisea); Chrysosporium sp. (e.g., C. lucknowense), Gliocladium sp., Aspergillus sp. (e.g., A. oryzae, A. niger, and A. awamori), Fusarium sp., Neurospora sp., Hypocrea sp., and Emericella sp. (See also, Innis et al., (1985) Sci. 228:21-26).

As used herein, the term “Trichoderma” or “Trichoderma sp.” refer to any fungal genus previously or currently classified as Trichoderma.

An expression cassette can be included in the production host, particularly in the cells of microbial production hosts. The production host cells can be microbial hosts found within the fungal families and which grow over a wide range of temperature, pH values, and solvent tolerances. For example, it is contemplated that any of bacteria, yeast, plants, algae, or fungi such as filamentous fungi, may suitably host the expression vector.

Inclusion of the expression cassette in the production host cell may be used to express the protein of interest so that it may reside intracellularly, extracellularly, or a combination of both inside and outside the cell. Extracellular expression renders recovery of the desired protein from a fermentation product more facile than methods for recovery of protein produced by intracellular expression.

Methods for transforming nucleic acids into filamentous fungi such as Aspergillus spp., e.g., A. oryzae or A. niger, H. grisea, H. insolens, and T. reesei. arc well known in the art. A suitable procedure for transformation of Aspergillus host cells is described, for example, in EP238023.

A suitable procedure for transformation of Trichoderma host cells is described, for example, in Steiger et al 2011, Appl. Environ. Microbiol. 77:114-121. Uptake of DNA into the host Trichoderma sp. strain is dependent upon the calcium ion concentration. Generally, between about 10 mM CaCl₂) and 50 mM CaCl₂) is used in an uptake solution. Besides the need for the calcium ion in the uptake solution, other compounds generally included are a buffering system such as TE buffer (10 Mm Tris, pH 7.4; 1 mM EDTA) or 10 mM MOPS, pH 6.0 buffer (morpholinepropanesulfonic acid) and polyethylene glycol (PEG). It is believed that the polyethylene glycol acts to fuse the cell membranes, thus permitting the contents of the medium to be delivered into the cytoplasm of the Trichoderma sp. strain and the plasmid DNA is transferred to the nucleus. This fusion frequently leaves multiple copies of the plasmid DNA integrated into the host chromosome.

Usually a suspension containing the Trichoderma sp. protoplasts or cells that have been subjected to a permeability treatment at a density of 105 to 107/mL, preferably 2×10⁶/mL are used in transformation. A volume of 100 μL of these protoplasts or cells in an appropriate solution (e.g., 1.2 M sorbitol; 50 mM CaCl₂)) are mixed with the desired DNA. Generally, a high concentration of PEG is added to the uptake solution. From 0.1 to 1 volume of 25% PEG 4000 can be added to the protoplast suspension. However, it is preferable to add about 0.25 volumes to the protoplast suspension. Additives such as dimethyl sulfoxide, heparin, spermidine, potassium chloride and the like may also be added to the uptake solution and aid in transformation. Similar procedures are available for other fungal host cells. (see, e.g., U.S. Pat. Nos. 6,022,725 and 6,268,328, both of which are incorporated by reference).

Preferably, genetically stable transformants are constructed with vector systems whereby the nucleic acid encoding the phytase polypeptide or fragment thereof is stably integrated into a host strain chromosome. Transformants are then purified by known techniques.

After the expression vector is introduced into the cells, the transfected or transformed cells are cultured under conditions favoring expression of genes under control of the promoter sequences.

Generally, cells are cultured in a standard medium containing physiological salts and nutrients (see, e.g., Pourquic, J. ct al., BIOCHEMISTRY AND GENETICS OF CELLULOSE DEGRADATION, eds. Aubert, J. P. et al., Academic Press, pp. 71-86, 1988 and IImen, M. et al., (1997) Appl. Environ. Microbiol. 63:1298-1306). Common commercially prepared media (e.g., Yeast Malt Extract (YM) broth, Luria Bertani (LB) broth and Sabouraud Dextrose (SD) broth also find use in the present invention.

Culture-conditions are also standard, (e.g., cultures are incubated at approximately 28 °C in appropriate medium in shake cultures or fermenters until desired levels of phytase expression are achieved). Preferred culture conditions for a given filamentous fungus are known in the art and may be found in the scientific literature and/or from the source of the fungi such as the American Type Culture Collection and Fungal Genetics Stock Center.

After fungal growth has been established, the cells are exposed to conditions effective to cause or permit the expression of a phytase and particularly a phytase as defined herein. In cases where a phytase coding sequence is under the control of an inducible promoter, the inducing agent (e.g., a sugar, metal salt or antimicrobial), is added to the medium at a concentration effective to induce phytase expression. An engineered phytase polypeptide or fragment thereof secreted from the host cells can be used, with minimal post-production processing, as a whole broth preparation.

The preparation of a spent whole fermentation broth of a recombinant microorganism can be achieved using any cultivation method known in the art resulting in the expression of an engineered phytase polypeptide or fragment thereof.

The term “spent whole fermentation broth” is defined herein as unfractionated contents of fermentation material that includes culture medium, extracellular proteins (e.g., enzymes), and cellular biomass. It is understood that the term “spent whole fermentation broth” also encompasses cellular biomass that has been lysed or permeabilized using methods well known in the art.

After fermentation, a fermentation broth is obtained, the microbial cells and various suspended solids, including residual raw fermentation materials, are removed by conventional separation techniques in order to obtain a phytase solution. Filtration, centrifugation, microfiltration, rotary vacuum drum filtration, ultrafiltration, centrifugation followed by ultrafiltration, extraction, or chromatography, or the like, are generally used.

It is possible to optionally recover the desired protein from the production host. In another aspect, an enginecred phytase polypeptide or fragment thereof containing culture supernatant is obtained by using any of the methods known to those skilled in the art.

Examples of these techniques include, but are not limited to, affinity chromatography (Tilbeurgh et a., (1984) FEBS Lett. 16:215), ion-exchange chromatographic methods (Goyal et al., (1991) Biores. Technol. 36:37; Fliess et al., (1983) Eur. J. Appl. Microbiol. Biotechnol. 17:314; Bhikhabhai et al, (1984) J. Appl. Biochem. 6:336; and Ellouz et al., (1987) Chromatography 396:307), including ion-exchange using materials with high resolution power (Medve et al., (1998) J. Chromatography A 808:153), hydrophobic interaction chromatography (See, Tomaz and Queiroz, (1999) J. Chromatography A 865:123; two-phase partitioning (See, Brumbauer, et al., (1999) Bioseparation 7:287); ethanol precipitation; reverse phase HPLC, chromatography on silica or on a cation-exchange resin such as DEAE, chromatofocusing, SDS-PAGE, ammonium sulfate precipitation, and gel filtration (e.g., Sephadex G-75). The degree of purification desired will vary depending on the use of the engineered phytase polypeptide or fragment thereof. In some embodiments, purification will not be necessary.

On the other hand, it may be desirable to concentrate a solution containing an engineered phytase polypeptide or fragment thereof in order to optimize recovery. Use of unconcentrated solutions requires increased incubation time in order to collect the enriched or purified enzyme precipitate. The enzyme containing solution is concentrated using conventional concentration techniques until the desired enzyme level is obtained. Concentration of the enzyme containing solution may be achieved by any of the techniques discussed herein. Exemplary methods of enrichment and purification include but are not limited to rotary vacuum filtration and/or ultrafiltration.

In addition, concentration of the desired protein product may be performed using, e.g., a precipitation agent, such as a metal halide precipitation agent. The metal halide precipitation agent, sodium chloride, can also be used as a preservative. The metal halide precipitation agent is used in an amount effective to precipitate the engineered phytase polypeptide or fragment thereof. The selection of at least an effective amount and an optimum amount of metal halide effective to cause precipitation of the enzyme, as well as the conditions of the precipitation for maximum recovery including incubation time, pH, temperature and concentration of enzyme, will be readily apparent to one of ordinary skill in the art, after routine testing. Generally, at least about 5% w/v (weight/volume) to about 25% w/v of metal halide is added to the concentrated enzyme solution, and usually at least 8% w/v.

Another alternative way to precipitate the enzyme is to use organic compounds. Exemplary organic compound precipitating agents include: 4-hydroxybenzoic acid, alkali metal salts of 4-hydroxybenzoic acid, alkyl esters of 4-hydroxybenzoic acid, and blends of two or more of these organic compounds. The addition of the organic compound precipitation agents can take place prior to, simultaneously with or subsequent to the addition of the metal halide precipitation agent, and the addition of both precipitation agents, organic compound and metal halide, may be carried out sequentially or simultaneously. Generally, the organic precipitation agents are selected from the group consisting of alkali metal salts of 4-hydroxybenzoic acid, such as sodium or potassium salts, and linear or branched alkyl esters of 4-hydroxybenzoic acid, wherein the alkyl group contains from 1 to 12 carbon atoms, and blends of two or more of these organic compounds. Additional organic compounds also include but are not limited to 4-hydroxybenzoic acid methyl ester (named methyl PARABEN), 4-hydroxybenzoic acid propyl ester (named propyl PARABEN). For further descriptions, see, e.g., U.S. Pat. No. 5,281,526. Addition of the organic compound precipitation agent provides the advantage of high flexibility of the precipitation conditions with respect to pH, temperature, concentration, precipitation agent, protein concentration, and time of incubation. Generally, at least about 0.01% w/v and no more than about 0.3% w/v of organic compound precipitation agent is added to the concentrated enzyme solution.

After the incubation period, the enriched or purified enzyme is then separated from the dissociated pigment and other impurities and collected by conventional separation techniques, such as filtration, centrifugation, microfiltration, rotary vacuum filtration, ultrafiltration, press filtration, cross membrane microfiltration, cross flow membrane microfiltration, or the like. Further enrichment or purification of the enzyme precipitate can be obtained by washing the precipitate with water. For example, the enriched or purified enzyme precipitate is washed with water containing the metal halide precipitation agent, or with water containing the metal halide and the organic compound precipitation agents.

Sometimes it is advantageous to delete genes from expression hosts, where the gene deficiency can be cured by an expression vector. Where it is desired to obtain a fungal host cell having one or more inactivated genes known methods may be used (e.g, methods disclosed in U.S. Pat. No. 5,246,853, U.S. Pat. No. 5,475,101 and WO92/06209). Gene inactivation may be accomplished by complete or partial deletion, by insertional inactivation or by any other means which renders a gene nonfunctional for its intended purpose (such that the gene is prevented from expression of a functional protein).

Any gene from a Trichoderma sp. or other filamentous fungal host, which has been cloned can be deleted, for example cbh1, cbh2, egl1 and egl2 genes. In some embodiments, gene deletion may be accomplished by inserting a form of the desired gene to be inactivated into a plasmid by methods known in the art. The deletion plasmid is then cut at an appropriate restriction enzyme site(s), internal to the desired gene coding region, and the gene coding sequence or part thereof is replaced with a selectable marker. Flanking DNA sequences from the locus of the gene to be deleted (preferably between about 0.5 to 2.0 kb) remain on either side of the marker gene. An appropriate deletion plasmid will generally have unique restriction enzyme sites present therein to enable the fragment containing the deleted gene, including the flanking DNA sequences and the selectable markers gene to be removed as a single linear piece.

Depending upon the host cell used post-transcriptional and/or post-translational modifications may be made. One non-limiting example of a post-transcriptional and/or post-translational modification is “clipping” or “truncation” of a polypeptide. In another instance, this clipping may result in taking a mature phytase polypeptide and further removing N or C-terminal amino acids to generate truncated forms of the phytase that retain enzymatic activity.

Other examples of post-transcriptional or post-translational modifications include, but are not limited to, myristoylation, glycosylation, truncation, lipidation and tyrosine, serine or threonine phosphorylation. T The skilled person will appreciate that the type of post-transcriptional or post-translational modifications that a protein may undergo may depend on the host organism in which the protein is expressed.

Further sequence modifications of polypeptides post expression may occur. This includes, but is not limited to, oxidation, deglycosylation, glycation, etc. It is known that glycation can affect the activity of phytase when subjected to incubation with glucose or other reducing sugars especially at temperatures above 30 °C and neutral or alkaline pH. Protein engineering to eliminate Lysine residues can be used to prevent such modification. An example of this can be found in U.S. Pat. No. 8,507,240. For example, yeast expression can result in highly glycosylated polypeptides resulting in an apparent increased molecular weight. Also, WO2013/119470 (incorporated by reference herein) having international publication date Aug. 15, 2013 relates to phytases having increased stability believed to be due to increased glycosylation.

The term “glycosylation” as used herein refers to the attachment of glycans to molecules, for example to proteins. Glycosylation may be an enzymatic reaction. The attachment formed may be through covalent bonds. The phrase “highly glycosylated” refers to a molecule such as an enzyme which is glycosylated in many sites and at all or nearly all the available glycosylation sites, for instance N-linked glycosylation sites. Alternatively, or in addition to, the phrase “highly glycosylated” can refer to extensive glycolytic branching (such as, the size and number of glycolytic moieties associated with a particular N-linked glycosylation site) at all or substantially all N-linked glycosylation sites. In some embodiments, the engineered phytase polypeptide is glycosylated at all or substantially all consensus N-linked glycosylation sites (i.e. an NXS/T consensus N-linked glycosylation site).

The term “glycan” as used herein refers to a polysaccharide or oligosaccharide, or the carbohydrate section of a glycoconjugate such as a glycoprotein. Glycans may be homo- or heteropolymers of monosaccharide residues. They may be linear or branched molecules.

A phytase may have varying degrees of glycosylation. It is known that such glycosylations may improve stability during storage and in applications. Extensive

The activity of any of the engineered phytase polypeptides or fragments thereof disclosed herein can be determined as discussed above.

It is believed that applying a robust engineered phytase polypeptide or fragment thereof to feed in a liquid form is beneficial as compared to applying such a phytase as a coated granule. This coated granule is the current commercial approach to make phytase products suitable for high temperature conditioning and pelleting. Benefits of liquid application of robust enzyme include; 1) the enzyme will start to work immediately after ingestion by an animal since it does not have to be released from the coated granule before it can interact with the feed, 2) there is improved distribution of the enzyme throughout the feed, thus, ensuring a more consistent delivery of the enzyme to the animal which is particularly important for young animals that eat small amounts of feed, 3) even distribution in the feed makes it easier to measure the enzyme in the feed, and 4) in the case of a robust phytase, such as the engineered phytase polypeptide and fragment disclosed herein, it may start to degrade phytate already present in the feed.

In other words, the novel engineered phytase polypeptides and fragments thereof are so robust that no special coating or formulation is believed to be needed to apply them to feed prior to conditioning and pelleting since they have been engineered to withstand the stress of conditioning and pelleting used in industrial feed production. Accordingly, the robustness of the novel engineered phytase polypeptides and fragments thereof described herein is such that they can be applied as an uncoated granule or particle or uncoated and unprotected when put into a liquid.

It should be noted that the engineered phytase polypeptides and fragments thereof can be formulated inexpensively on a solid carrier without specific need for protective coatings and still maintain activity throughout the conditioning and pelleting process. A protective coating to provide additional thermostability when applied in a solid form can be beneficial for obtaining pelleting stability when required in certain regions where harsher conditions are used or if conditions warrant it, e.g., as in the case of super conditioning feed above 90 °C.

The disclosed engineered phytase polypeptides or fragments thereof were derived using a combination of methods and techniques know in the field of protein engineering which include, phylogenetic analysis, site evaluation libraries, combinatorial libraries, high throughput screening and statistical analysis.

In one aspect, the disclosure relates to an engineered phytase polypeptide or fragment thereof also that has at least 82% sequence identity with the amino acid sequence of SEQ ID NO: 1.

Those skilled in the art will appreciate that such at least 82% sequence identity also includes 82%, 83%, 84%, 85%, 86%, 87%, 88%, 89%, 90%, 91%, 92%, 93%, 94%, 95%, 96%, 97%, 98%, 99% or 100%.

Those skilled in the art will appreciate that at least 79% sequence identity also includes 79%, 80%, 81%, 82%, 83%, 84%, 85%, 86%, 87%, 88%, 89%, 90%, 91%, 92%, 93%, 94%, 95%, 96%, 97%, 98%, 99% or 100%.

There can also be mentioned the following in that in some embodiments, there is provided:
a) an engineered phytase polypeptide or fragment thereof also that has at least 81% (such as 81%, 82%, 83%, 84%, 85%, 86%, 87%, 88%, 89%, 90%, 91%, 92%, 93%, 94%, 95%, 96%, 97%, 98%, 99% or 100%) sequence identity with the amino acid sequence of SEQ ID NOs: 2, 3, 8, 10, 12, 18, 19, 24, 26, 27, 28, 30, 31, 32, 33, and/or 36.
b) an engineered phytase polypeptide or fragment thereof also that has at least 82% (such as 82%, 83%, 84%, 85%, 86%, 87%, 88%, 89%, 90%, 91%, 92%, 93%, 94%, 95%, 96%, 97%, 98%, 99% or 100%) sequence identity with the amino acid sequence of SEQ ID NOs: 1, 4, 5, 7, 9, 11, 14, 15, 17, 21, 25, 34, and/or 35;
c) an engineered phytase polypeptide or fragment thereof also that has at least 83% (such as, 83%, 84%, 85%, 86%, 87%, 88%, 89%, 90%, 91%, 92%, 93%, 94%, 95%, 96%, 97%, 98%, 99% or 100%) sequence identity with the amino acid sequence of SEQ ID NO:13;
d) an engineered phytase polypeptide or fragment thereof also that has at least 79% (such as, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity with the amino acid sequence of SEQ ID NOs: 6, and/or 22; and/or
e) an engineered phytase polypeptide or fragment thereof also that has at least 80% (such as, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity with the amino acid sequence of SEQ ID NOs: 16, 20, 23, 29, and/or 37.

In further aspects, the polypeptide comprises a core domain of an engineered phytase polypeptide or is a core domain fragment of an engineered phytase polypeptide. A “core domain fragment” is herein defined as a polypeptide having one or more amino acids deleted from the amino and/or carboxyl terminus of the polypeptide. As used herein, the phrase “core domain” refers to a polypeptide region encompassing amino acids necessary to maintain the structure and function (such as, phytic acid hydrolysis) of the polypeptide. Amino acids in the core domain can be further modified to improve thermostability or catalytic activity under various conditions such as, without limitation, pH. In some non-limiting embodiments, the core domain of the engineered phytase polypeptides or fragment thereof disclosed herein corresponds to amino acid positions 14-325 of SEQ ID NO:1. In other non-limiting embodiments, the core domain corresponds to amino acid positions 13-326, 12-327, 11-328, 10-329, 9-330, 8-331, 7-332, 6-333, 5-334, 4-335, 3-336, 2-337, or 1-338 of SEQ ID NO:1. In other embodiments, the N-terminus of the core domain corresponds to amino acid position 1, 2, 3, 4, 5, 6, 7, 8, 9, 10, 11, 12, 13, 14, 15, 16, 17, 18, 19, 20, 21, 22, 23, 24, or 25 of SEQ ID NO:1 and the C-terminus of the core domain corresponds to amino acid position 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, or 413 of SEQ ID NO:1.

Accordingly, also provided herein are:
f) an engineered phytase polypeptide or core domain fragment thereof that has at least 78% (such as, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to amino acids 14-325 of SEQ ID NO:6, wherein said amino acid positions correspond to those of SEQ ID NO:1;
g) an engineered phytase polypeptide or core domain fragment thereof that has at least 79% (such as, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to amino acids 14-325 of SEQ ID NOs: 2, 8, 27, and/or 37, wherein said amino acid positions correspond to those of SEQ ID NO: 1;
h) an engineered phytase polypeptide or core domain fragment thereof that has at least 81% (such as, 81%, 82%, 83%, 84%, 85%, 86%, 87%, 88%, 89%, 90%, 91%, 92%, 93%, 94%, 95%, 96%, 97%, 98%, 99% or 100%) sequence identity to amino acids 14-325 of SEQ ID NOs: 3, 10, 12, 18, 25, 26, 28, 30, 32, and/or 35, wherein said amino acid positions correspond to those of SEQ ID NO:1;
i) an engineered phytase polypeptide or core domain fragment thereof that has at least 82% (such as, 82%, 83%, 84%, 85%, 86%, 87%, 88%, 89%, 90%, 91%, 92%, 93%, 94%, 95%, 96%, 97%, 98%, 99% or 100%) sequence identity to amino acids 14-325 of SEQ ID NOs: 1, 4, 5, 7, 9, 11, 13-17, 21, 22, 31, 33, 34, and/or 36, wherein said amino acid positions correspond to those of SEQ ID NO:1;
j) an engineered phytase polypeptide or core domain fragment thereof that has at least 83% (such as, 83%, 84%, 85%, 86%, 87%, 88%, 89%, 90%, 91%, 92%, 93%, 94%, 95%, 96%, 97%, 98%, 99% or 100%) sequence identity to amino acids 14-325 of SEQ ID NOs: 19, 20, 23, and/or 24, wherein said amino acid positions correspond to those of SEQ ID NO:1; and/or
k) an engineered phytase polypeptide or core domain fragment thereof that has at least 84% (such as, 84%, 85%, 86%, 87%, 88%, 89%, 90%, 91%, 92%, 93%, 94%, 95%, 96%, 97%, 98%, 99% or 100%) sequence identity to amino acids 14-325 of SEQ ID NO:29, wherein said amino acid positions correspond to those of SEQ ID NO:1.

In another aspect, any of the engineered polypeptides or fragments thereof disclosed herein comprise a specific activity of at least about 100 U/mg at pH 3.5. The specific activity range (U/mg at pH 3.5) includes, but is not limited to, about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 2000, etc.

In another aspect, some of the engineered polypeptides or fragments thereof disclosed herein comprise a specific activity of at least about 100 U/mg at pH 5.5. The specific activity range (U/mg at pH 5.5) includes, but is not limited to, about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 2000, etc.

In still another aspect, any of the engineered phytase polypeptides or fragments thereof disclosed herein may be stable in a liquid form at a pH about 3.0 or lower. This is very relevant when engineered phytase polypeptides or fragments thereof described herein are passing through the digestive tract of an animal as is discussed below.

In another embodiment, there is described non-inorganic phosphate-containing diet comprising any of the engineered phytase polypeptides or fragments thereof described herein. Importantly, feed additive enzymes e.g. a phytase is subjected to very harsh conditions as it passes through the digestive track of an animal, i.e. low pH and presence of digestive enzymes. Pepsin is one of the most important proteolytic digestive enzymes present in the gastrointestinal tract of monogastric animals. Pepsin has low specificity and high pH tolerance in the acidic area (pH 1.5-6.0 stabile up to pH 8.0). The engineered phytase polypeptides or fragments thereof described herein are largely resistant against pepsin, which is necessary for good in-vivo performance.

The non-inorganic phosphate-containing diets comprising any of the engineered phytase polypeptides or fragments thereof described herein may be used (i) alone or (ii) with at least one other enzyme or (iii) further comprising at least one other feed additive component and, optionally, the engineered phytase polypeptide or fragment thereof is present in an amount of at least 0.1 g/ton feed (such as at least about 0.1 g/ton, 0.2 g/ton, 0.3 g/ton, 0.4 g/ton, 0.5 g/ton, 0.6 g/ton, 0.7 g/ton, 0.8 g/ton, 0.9 g/ton, 1 g/ton, 1.1 g/ton, 1.2 g/ton, 1.3 g/ton, 1.4 g/ton, 1.5 g/ton, 1.6 g/ton, 1.7 g/ton, 1.8 g/ton, 1.9 g/ton, 2 g/ton, 2.1 g/ton, 2.2 g/ton, 2.3 g/ton, 2.4 g/ton, 2.5 g/ton, 2.6 g/ton, 2.7 g/ton, 2.8 g/ton, 2.9 g/ton, 3 g/ton, or more).

In some non-limiting embodiments, the phytase is present in the diet in range of about 500 FTU/kg to about 7000 FTU/kg feed or about 1000 FTU/kg feed to about 5000 FTU/kg feed. In one embodiment, the phytase is present in the feedstuff at more than about 200 FTU/kg feed, suitably more than about 300 FTU/kg feed, suitably more than about 400 FTU/kg feed, suitably more than about 500 FTU/kg feed, suitably more than about 600 FTU/kg feed, suitably more than about 700 FTU/kg feed, suitably more than about 800 FTU/kg feed, suitably more than about 900 FTU/kg feed, suitably more than about 400 FTU/kg feed, suitably more than about 1000 FTU/kg feed, suitably more than about 1100 FTU/kg feed, suitably more than about 1200 FTU/kg feed, suitably more than about 1300 FTU/kg feed, suitably more than about 1400 FTU/kg feed, suitably more than about 1500 FTU/kg feed, suitably more than about 1600 FTU/kg feed, suitably more than about 1700 FTU/kg feed, suitably more than about 1800 FTU/kg feed, suitably more than about 1900 FTU/kg feed, suitably more than about 2000 FTU/kg feed, suitably more than about 2100 FTU/kg feed, suitably more than about 2200 FTU/kg feed, suitably more than about 2300 FTU/kg feed, suitably more than about 2400 FTU/kg feed, suitably more than about 2500 FTU/kg feed, suitably more than about 2600 FTU/kg feed, suitably more than about 2700 FTU/kg feed, suitably more than about 2800 FTU/kg feed, suitably more than about 2900 FTU/kg feed, suitably more than about 3000 FTU/kg feed suitably more than about 3100 FTU/kg feed, suitably more than about 3200 FTU/kg feed, suitably more than about 3300 FTU/kg feed, suitably more than about 3400 FTU/kg feed, suitably more than about 3500 FTU/kg feed, suitably more than about 3600 FTU/kg feed, suitably more than about 3700 FTU/kg feed, suitably more than about 3800 FTU/kg feed, suitably more than about 3900 FTU/kg feed, suitably more than about 4000 FTU/kg feed, suitably more than about 4100 FTU/kg feed, suitably more than about 4200 FTU/kg feed, suitably more than about 4300 FTU/kg feed, suitably more than about 4400 FTU/kg feed, suitably more than about 4500 FTU/kg feed, suitably more than about 4600 FTU/kg feed, suitably more than about 4700 FTU/kg feed, suitably more than about 4800 FTU/kg feed suitably more than about 4900 FTU/kg feed, suitably more than about 5000 FTU/kg feed, suitably more than about 5100 FTU/kg feed, suitably more than about 5200 FTU/kg fecd, suitably more than about 5300 FTU/kg feed, suitably more than about 5400 FTU/kg feed, suitably more than about 5500 FTU/kg feed, suitably more than about 5600 FTU/kg feed, suitably more than about 5700 FTU/kg feed.

In some non-limiting embodiments, “1 FTU” (phytase unit) is defined as the amount of enzyme required to release 1 μmol of inorganic orthophosphate from a substrate in one minute under the reaction conditions defined in the ISO 2009 phytase assay-A standard assay for determining phytase activity and 1 FTU can be found at International Standard ISO/DIS 30024:1-17, 2009. In one embodiment, the enzyme is classified using the E.C. classification above, and the E.C. classification designates an enzyme having that activity when tested in the assay taught herein for determining 1 FTU.

The terms “feed additive”, “feed additive components”, and/or “feed additive ingredients” are used interchangeably herein.

Feed additives can be described as products used in animal nutrition for purposes of improving the quality of feed and the quality of food from animal origin, or to improve the animals' performance and health, e.g. providing enhanced digestibility of the feed materials.

Feed additives fall into a number of categories such as sensory additives which stimulate an animal's appetite so that they naturally want to eat more. Nutritional additives provide a particular nutrient that may be deficient in an animal's diet. Zootechnical additives improve the overall nutritional value of an animal's diet through additives in the feed.

As used herein, a “non-inorganic phosphate-containing diet” refers to a diet that contains no to substantially no (such as reduced) exogenously added inorganic phosphate, for example, as a feed additive. By removal of supplementation of inorganic phosphate, the total P content in the diet may be reduced by about 10-15% (such as any of about 10%, 11%, 12%, 13%, 14%, or 15%) compared to diets that are supplemented with exogenously added inorganic phosphate.

The term “phosphorus deficient diet” or a “diet having low phosphorous content” refers to a diet containing lower levels of the mineral than required for optimal growth. If the diet lacks phosphorus that would lead a phosphorus deficiency in the body of animal, calcium will also not be retained by the animal. Excess Ca can lead to poor phosphorus (P) digestibility and contribute to the formation of insoluble mineral-phytate complexes. Both deficiency of P and Ca can cause reduced skeletal integrity, subnormal growth and ultimately weight loss.

“Inorganic phosphorous” and “inorganic phosphate” are used interchangeably herein to denote dietary supplements commonly added to animal feed to ensure the animal receives sufficient phosphate to satisfy the nutritional requirements of an animal. The diets disclosed herein, however, contain no or substantially no inorganic phosphate when said diets also include the engineered phytase polypeptides disclosed herein at proper dose with diets containing sufficient phytate as substrate. The expression “substantially none” or “substantially no” as used herein to describe the amount of inorganic phosphate in the diet formulations disclosed herein, means that any amounts that are present are either trace amounts, amounts included unintentionally, and/or amounts that that are less than about 0.1% in the diets.

Feed additive compositions or formulations may also comprise at least one component selected from the group consisting of a protein, a peptide, sucrose, lactose, sorbitol, glycerol, propylene glycol, sodium chloride, sodium sulfate, sodium acetate, sodium citrate, sodium formate, sodium sorbate, potassium chloride, potassium sulfate, potassium acetate, potassium citrate, potassium formate, potassium acetate, potassium sorbate, magnesium chloride, magnesium sulfate, magnesium acetate, magnesium citrate, myo-inositol, magnesium formate, magnesium sorbate, sodium metabisulfite, methyl paraben and propyl paraben.

At least one other enzyme (i.e. in addition to any of the engineered phytase polypeptides or fragments thereof disclosed herein) can be included in the feed additive compositions or formulations disclosed herein which can include, but are not limited to, a xylanase, amylase, another phytase, beta-glucanase, and/or a protease.

Xylanase is the name given to a class of enzymes that degrade the linear polysaccharide β-1,4-xylan into xylose, thus breaking down hemicellulose, one of the major components of plant cell walls. Xylanases, e.g., endo-β-xylanases (EC 3.2.1.8) hydrolyze the xylan backbone chain.

In one embodiment, the xylanase may be any commercially available xylanase. Suitably the xylanase may be an endo-1,4-P-d-xylanase (classified as E.G. 3.2.1.8) or a 1,4β-xylosidase (classified as E.G. 3.2.1.37). In one embodiment, the disclosure relates to a composition comprising any of the engineered phytase polypeptides or fragments thereof disclosed herein in combination with an endoxylanase, e.g. an endo-1,4-P-d-xylanase, and another enzyme. All E.C. enzyme classifications referred to herein relate to the classifications provided in Enzyme Nomenclature-Recommendations (1992) of the nomenclature committee of the International Union of Biochemistry and Molecular Biology—ISBN 0-12-226164-3, which is incorporated herein.

In another embodiment, the xylanase may be a xylanase from Bacillus, Trichodermna, Therinomyces, Aspergillus, Humicola and Penicillium. In still another embodiment, the xylanase may be the xylanase in Axtra XAP® or Avizyme 1502®, both commercially available products from Danisco A/S. In one embodiment, the xylanase may be a mixture of two or more xylanases. In still another embodiment, the xylanase is an endo-1,4-β-xylanase or a 1,4-β-xylosidase.

In one embodiment, the disclosure relates to a non-inorganic phosphate-containing diet comprising any of the engineered phytase polypeptides or fragments thereof disclosed herein and a xylanase. In one embodiment, the non-inorganic phosphate-containing diet comprises 10-50, 50-100, 100-150, 150-200, 200-250, 250-300, 300-350, 350-400, 400-450, 450-500, 500-550, 550-600, 600-650, 650-700, 700-750, and greater than 750 xylanase units/g of composition.

In one embodiment, the non-inorganic phosphate-containing diet comprises 500-1000, 1000-1500, 1500-2000, 2000-2500, 2500-3000, 3000-3500, 3500-4000, 4000-4500, 4500-5000, 5000-5500, 5500-6000, 6000-6500, 6500-7000, 7000-7500, 7500-8000, and greater than 8000 xylanase units/g composition.

It will be understood that one xylanase unit (XU) is the amount of enzyme that releases 0.5 μmol of reducing sugar equivalents (as xylose by the Dinitrosalicylic acid (DNS) assay-reducing sugar method) from an oat-spelt-xylan substrate per min at pH 5.3 and 50 °C (Bailey, et al., Journal of Biotechnology, Volume 23, (3), May 1992, 257-270).

Amylase is a class of enzymes capable of hydrolysing starch to shorter-chain oligosaccharides, such as maltose. The glucose moiety can then be more easily transferred from maltose to a monoglyceride or glycosylmonoglyceride than from the original starch molecule. The term amylase includes a-amylases (E.C. 3.2.1.1), G4-forming amylases (E.C. 3.2.1.60), β-amylases (E.C. 3.2.1.2) and γ-amylases (E.C. 3.2.1.3). Amylases may be of bacterial or fungal origin, or chemically modified or protein engineered mutants.

In one embodiment, the amylase may be a mixture of two or more amylases. In another embodiment, the amylase may be an amylase, e.g. an a-amylase, from Bacillus licheniformis and an amylase, e.g. an α-amylase, from Bacillus amyloliquefaciens. In one embodiment, the α-amylase may be the α-amylase in Axtra XAP® or Avizyme 1502®, both commercially available products from Danisco A/S. In yet another embodiment, the amylase may be a pepsin resistant α-amylase, such as a pepsin resistant Trichoderma (such as Trichoderma reesei) alpha amylase. A suitably pepsin resistant α-amylase is taught in UK application number 101 1513.7 (which is incorporated herein by reference) and PCT/IB2011/053018 (which is incorporated herein by reference).

It will be understood that one amylase unit (AU) is the amount of enzyme that releases 1 mmol of glucosidic linkages from a water insoluble cross-linked starch polymer substrate per min at pH 6.5 and 37 °C (this may be referred to herein as the assay for determining 1 AU).

In one embodiment, disclosure relates to a non-inorganic phosphate-containing diet comprising any of the engineered phytase polypeptides or fragments thereof disclosed herein and an amylase. In one embodiment, disclosure relates to a non-inorganic phosphate-containing diet comprising any of the engineered phytase polypeptides or fragments thereof disclosed herein, xylanase and amylase. In one embodiment, the composition comprises 10-50, 50-100, 100-150, 150-200, 200-250, 250-300, 300-350, 350-400, 400-450, 450-500, 500-550, 550-600, 600-650, 650-700, 700-750, and greater than 750 amylase units/g composition.

In one embodiment, the non-inorganic phosphate-containing diet comprises 500-1000, 1000-1500, 1500-2000, 2000-2500, 2500-3000, 3000-3500, 3500-4000, 4000-4500, 4500-5000, 5000-5500, 5500-6000, 6000-6500, 6500-7000, 7000-7500, 7500-8000, 8000-8500, 8500-9000, 9000-9500, 9500-10000, 10000-11000, 11000-12000, 12000-13000, 13000-14000, 14000-15000 and greater than 15000 amylase units/g composition.

The term protease as used herein is synonymous with peptidase or proteinase. The protease may be a subtilisin (E.C. 3.4.21.62) or a bacillolysin (E.C. 3.4.24.28) or an alkaline serine protease (E.C. 3.4.21.x) or a keratinase (E.C. 3.4.X.X). In one embodiment, the protease is a subtilisin. Suitable proteases include those of animal, vegetable or microbial origin.

Chemically modified or protein engineered mutants are also suitable. The protease may be a serine protease or a metalloprotease. e.g., an alkaline microbial protease or a trypsin-like protease. In one embodiment, provided herein are compositions comprising any of the engineered phytase polypeptides or fragments thereof disclosed herein and one or more protease.

Examples of alkaline proteases are subtilisins, especially those derived from Bacillus sp., e.g., subtilisin Novo, subtilisin Carlsberg, subtilisin 309 (see, e.g., U.S. Pat. No. 6,287,841), subtilisin 147, and subtilisin 168 (see, e.g., WO 89/06279). Examples of trypsin-like proteases are trypsin (e.g., of porcine or bovine origin), and Fusarium proteases (see, e.g., WO 89/06270 and WO 94/25583). Examples of useful proteases also include but are not limited to the variants described in WO 92/19729 and WO 98/20115.

In one embodiment, the protease is selected from the group consisting of subtilisin, a bacillolysin, an alkine serine protease, a keratinase, and a Nocardiopsis protease.

It will be understood that one protease unit (PU) is the amount of enzyme that liberates from the substrate (0.6% casein solution) one microgram of phenolic compound (expressed as tyrosine equivalents) in one minute at pH 7.5 (40 mM Na₂PO₄/lactic acid buffer) and 40 °C. This may be referred to as the assay for determining 1 PU.

In one embodiment, disclosure relates to a non-inorganic phosphate-containing diet comprising any of the engineered phytase polypeptides or fragments thereof disclosed herein and a protease. In another embodiment, disclosure relates to a non-inorganic phosphate-containing diet comprising any of the engineered phytase polypeptides or fragments thereof disclosed herein and a xylanase and a protease. In still another embodiment, the disclosure relates to a non-inorganic phosphate-containing diet comprising any of the engineered phytase polypeptides or fragments thereof disclosed herein and an amylase and a protease. In yet another embodiment, the disclosure relates to a non-inorganic phosphate-containing diet comprising any of the engineered phytase polypeptides or fragments thereof disclosed herein and a xylanase, an amylase and a protease.

In one embodiment, the non-inorganic phosphate-containing diet comprises about 10-50, 50-100, 100-150, 150-200, 200-250, 250-300, 300-350, 350-400, 400-450, 450-500, 500-550, 550-600, 600-650, 650-700, 700-750, and greater than 750 protease units/g composition.

In one embodiment, the non-inorganic phosphate-containing diet comprises about 500-1000, 1000-1500, 1500-2000, 2000-2500, 2500-3000, 3000-3500, 3500-4000, 4000-4500, 4500-5000, 5000-5500, 5500-6000, 6000-6500, 6500-7000, 7000-7500, 7500-8000, 8000-8500, 8500-9000, 9000-9500, 9500-10000, 10000-11000, 11000-12000, 12000-13000, 13000-14000, 14000-15000 and greater than 15000 protease units/g composition.

In other embodiments, the diet can have reduced (such as substantially reduced) inorganic phosphate levels relative to total phosphorous levels recommended by the National Research Council (NRC) for ruminant animals (such as cattle, for example, dairy cows). In some embodiments, the diets contain from between 0.2% to about 75% inorganic phosphate levels relative to those recommended by the National Research Council (NRC) for ruminant animals (such as cattle, for example, dairy cows), such as any of about 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, or 75% inorganic phosphate levels relative to total phosphorus levels recommended by the National Research Council (NRC) for ruminant animals (such as cattle, for example, dairy cows).

In still another aspect, there is disclosed a non-inorganic phosphate-containing diet for use in animal feed comprising at least one polypeptide having phytase activity as described herein, used either alone or in combination with at least one direct fed microbial or in combination with at least one other enzyme or in combination with at least one direct fed microbial and at least one other enzyme, wherein the feed additive composition comprises may be in any form such as a granulated particle. Such granulated particles may be produced by a process selected from the group consisting of high shear granulation, drum granulation, extrusion, spheronization, fluidized bed agglomeration, fluidized bed spray coating, spray drying, freeze drying, prilling, spray chilling, spinning disk atomization, coacervation, tableting, or any combination of the above processes.

Furthermore, particles of the granulated feed additive composition can have a mean diameter of greater than 50 microns and less than 2000 microns

Those skilled in the art will understand that animal feed may include plant material such as corn, wheat, sorghum, soybean, canola, sunflower or mixtures of any of these plant materials or plant protein sources for poultry, pigs, ruminants, aquaculture and pets. It is contemplated that animal performance parameters, such as growth, feed intake and feed efficiency, but also improved uniformity, reduced ammonia concentration in the animal house and consequently improved welfare and health status of the animals will be improved.

Thus, there is disclosed a method for improving the nutritional value of an animal feed, wherein any of the engineered phytases or fragments thereof as described herein can be added to animal feed.

The phrase, an “effective amount” as used herein, refers to the amount of an active agent (such as, a phytase, e.g. any of the engineered phytase polypeptides disclosed herein) required to confer improved performance on an animal on one or more metrics, either alone or in combination with one or more other active agents (such as, without limitation, one or more additional enzyme(s), one or more DFM(s), one or more essential oils, etc.).

The term “animal performance” as used herein may be determined by any metric such as, without limitation, the feed efficiency and/or weight gain of the animal and/or by the feed conversion ratio and/or milk production levels and/or by the digestibility of a nutrient in a feed (e.g., amino acid digestibility or phosphorus digestibility) and/or digestible energy or metabolizable energy in a feed and/or by nitrogen retention and/or by animals' ability to avoid the negative effects of diseases or by the immune response of the subject.

Further provided herein are methods for improving phosphorous utilization in a dairy ruminant animal comprising administering to the animal a phytase polypeptide or a fragment thereof comprising phytase activity (such as a phytase polypeptide or a fragment thereof comprising at least 82% sequence identity to the amino acid sequence set forth in SEQ ID NO:1) As used herein, the phrase “improved phosphorous utilization” refers to an increased proportion of consumed phosphorus being retained in an animal's body and/or less phosphorus being excreted in the animal's in urine or feces. In some embodiments, the method results in about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 65%, 70%, 75%, 80%, 85%, 90%, 95%, 100%, 105%, 110%, 115%, 120%, or 125%, inclusive of all values falling in between these percentages, improvement in phosphorous utilization relative to phosphate utilization in a lactating ruminant animal that has not been administered the phytase polypeptide or a fragment thereof. In additional embodiments, the phytase polypeptide or fragment thereof is administered in conjunction with a diet having low phosphorous content (such as a diet that has not been supplemented with exogenously added inorganic phosphate). Phosphorous utilization can be measured using any means known in the art, including the methods described in the Examples section.

Further provided herein are methods for improving protein utilization in a lactating ruminant animal comprising administering to the animal a phytase polypeptide or a fragment thereof comprising phytase activity (such as a phytase polypeptide or a fragment thereof comprising at least 82% sequence identity to the amino acid sequence set forth in SEQ ID NO: 1). As used herein, the phrase “improved protein utilization” refers to an increased proportion of consumed protein being retained in the animal's body or secreted into milk protein and/or less protein (such as nitrogen) being excreted in the animal's in urine or feces. In some embodiments, the method results in about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 65%, 70%, 75%, 80%, 85%, 90%, 95%, 100%, 105%, 110%, 115%, 120%, or 125%, inclusive of all values falling in between these percentages, improvement in protein utilization relative to protein utilization in a lactating ruminant animal that has not been administered the phytase polypeptide or a fragment thereof. In additional embodiments, the phytase polypeptide or fragment thereof is administered in conjunction with a diet having low phosphorous content (such as a diet that has not been supplemented with exogenously added inorganic phosphate). Protein utilization can be measured using any means known in the art, including the methods described in the Examples section.

Also provided herein are methods for improving digestibility of crude protein in a lactating ruminant animal comprising administering to the animal a phytase polypeptide or a fragment thereof comprising phytase activity (such as a phytase polypeptide or a fragment thereof comprising at least 82% sequence identity to the amino acid sequence set forth in SEQ ID NO: 1). As used herein, the phrase “improved digestibility of crude protein” refers to an increased absorption of protein (such as amino acids) from the protein present in the animal's diet. In some embodiments, the method results in about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 65%, 70%, 75%, 80%, 85%, 90%, 95%, 100%, 105%, 110%, 115%, 120%, or 125%, inclusive of all values falling in between these percentages, improvement in digestibility of crude protein relative to the digestibility of crude protein in a lactating ruminant animal that has not been administered the phytase polypeptide or a fragment thereof. In additional embodiments, the phytase polypeptide or fragment thereof is administered in conjunction with a diet having low phosphorous content (such as a diet that has not been supplemented with exogenously added inorganic phosphate). Digestibility of crude protein can be measured using any means known in the art, including the methods described in the Examples section.

Additionally provided herein are methods for decreasing the excretion of crude protein in a lactating ruminant animal comprising administering to the animal a phytase polypeptide or a fragment thereof comprising phytase activity (such as a phytase polypeptide or a fragment thereof comprising at least 82% sequence identity to the amino acid sequence set forth in SEQ ID NO: 1). As used herein, the phrase “decreased excretion of crude protein” means a decrease in the amount of protein excreted in the feces and/or urine of the animal. In some embodiments, the method results in about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 65%, 70%, 75%, 80%, 85%, 90%, 95%, or 100%, inclusive of all values falling in between these percentages, decreased excretion of crude protein relative to the excretion of crude protein in a lactating ruminant animal that has not been administered the phytase polypeptide or a fragment thereof. In additional embodiments, the phytase polypeptide or fragment thereof is administered in conjunction with a diet having low phosphorous content (such as a diet that has not been supplemented with exogenously added inorganic phosphate). Excretion of crude protein can be measured using any means known in the art, including the methods described in the Examples section.

Further provided herein are methods for improving milk protein content in a lactating ruminant animal comprising administering to the animal a phytase polypeptide or a fragment thereof comprising phytase activity (such as a phytase polypeptide or a fragment thereof comprising at least 82% sequence identity to the amino acid sequence set forth in SEQ ID NO: 1). As used herein, the phrase “improved milk protein content” means the amount of protein content in the ruminant animal's milk is greater than that of a control group that has not been administered the phytase polypeptide or a fragment thereof. In some embodiments, the method results in about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 65%, 70%, 75%, 80%, 85%, 90%, 95%, 100%, 105%, 110%, 115%, 120%, or 125%, inclusive of all values falling in between these percentages, improved milk protein content relative to the milk protein content in a lactating ruminant animal that has not been administered the phytase polypeptide or a fragment thereof. In additional embodiments, the phytase polypeptide or fragment thereof is administered in conjunction with a diet having low phosphorous content (such as a diet that has not been supplemented with exogenously added inorganic phosphate). Milk protein content can be measured using any means known in the art, including the methods described in the Examples section.

Also provided herein are methods for improving milk protein yield in a lactating ruminant animal comprising administering to the animal a phytase polypeptide or a fragment thereof comprising phytase activity (such as a phytase polypeptide or a fragment thereof comprising at least 82% sequence identity to the amino acid sequence set forth in SEQ ID NO: 1). As used herein, the phrase “improved milk protein yield” means total milk protein production per day is higher and which is calculated as the product of milk protein x milk production/day. In some embodiments, the method results in about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 65%, 70%, 75%, 80%, 85%, 90%, 95%, 100%, 105%, 110%, 115%, 120%, or 125%, inclusive of all values falling in between these percentages, improved milk protein yield relative to the milk protein yield in a lactating ruminant animal that has not been administered the phytase polypeptide or a fragment thereof. In additional embodiments, the phytase polypeptide or fragment thereof is administered in conjunction with a diet having low phosphorous content (such as a diet that has not been supplemented with exogenously added inorganic phosphate). Milk protein yield can be measured using any means known in the art, including the methods described in the Examples section. Still further provided herein are methods for improving calcium digestibility in a lactating ruminant animal comprising administering to the animal a phytase polypeptide or a fragment thereof comprising phytase activity (such as a phytase polypeptide or a fragment thereof comprising at least 82% sequence identity to the amino acid sequence set forth in SEQ ID NO: 1). As used herein, the phrase “improved calcium digestibility” means an increased absorption of calcium relative to total calcium intake and corresponding less calcium excretion in the feces. In some embodiments, the method results in about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 65%, 70%, 75%, 80%, 85%, 90%, 95%, 100%, 105%, 110%, 115%, 120%, or 125%, inclusive of all values falling in between these percentages, improved calcium digestibility relative to the calcium digestibility in a lactating ruminant animal that has not been administered the phytase polypeptide or a fragment thereof. In additional embodiments, the phytase polypeptide or fragment thereof is administered in conjunction with a diet having low phosphorous content (such as a diet that has not been supplemented with exogenously added inorganic phosphate). Calcium digestibility can be measured using any means known in the art, including the methods described in the Examples section.

Ruminants have a stomach with four chambers, namely the rumen, reticulum, omasum and abomasum. In the first two chambers, the rumen and the reticulum, food is mixed with saliva and separates into layers of solid and liquid material. Solids clump together to form the cud, or bolus. The cud is then regurgitated, chewed slowly to completely mix it with saliva, which further breaks down fibers. Fiber, especially cellulose, is broken down into glucose in these chambers by the enzymes produced by commensal bacteria, protozoa and fungi. The broken-down fiber, which is now in the liquid part of the contents, then passes through the rumen and reticulum into the next stomach chamber, the omasum, where water is removed. The food in the abomasum is digested much like it would be in the human stomach. The abomasum has a pH of around 2.0 and therefore possesses an environment capable of denaturing most, if not all, polypeptides. The processed food is finally sent to the small intestine, where the absorption of the nutrients occurs.

As described in the Examples section, the inventors have surprisingly discovered that the phytase polypeptides disclosed herein, while being stable in the rumen, are also active in the abomasum compared to other phytases which are not as highly active in the abomasum. Phytate inhibits the hydrolysis of both starch and protein, leading it to be considered an anti-nutrient. Without being bound to theory, it is hypothesized that phytate-associated nutrients (for example, protein) broken down in the rumen are less nutritionally utilizable by the animal. In contrast, if not degraded in the rumen, but, rather, in the abomasum, then the products of phytase-mediated phytate dissolution are more likely to be available for the nutritional benefit of the animal.

As such, provided herein is a method for degrading phytate in the abomasum of a ruminant animal comprising administering to the animal any of the phytase polypeptides or functional fragments thereof provided herein (such as a phytase polypeptide or a fragment thereof comprising phytase activity (such as a phytase polypeptide or a fragment thereof comprising at least 82% sequence identity to the amino acid sequence set forth in SEQ ID NO: 1)). As used herein “degrading phytate in the abomasum of a ruminant animal” means a substantial amount (for example any of about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 75%, 80%, 85%, 90%, 95%, or 100%) of phytate is degraded in the abomasum of the animal compared to the total amount of degraded phytate in the diet and/or compared to the amount of phytate degraded in the rumen. The phytase polypeptide or functional fragment thereof can degrade phytase in the abomasum at about double the rate (such as any of about 1.5, 1.6, 1.7, 1.8, 1.9, 2, 2.1, 2.2, 2.3, 2.4, 2.5, 2.6, 2.7, 2.8, 2.9, or 3 times, or greater, the rate) of a control phytase polypeptide or a fragment thereof that does not comprise at least 82% sequence identity of the amino acid sequence set forth in SEQ ID NO:1. In other embodiments, the phytase polypeptide or functional fragment thereof can degrade phytase in the abomasum at about double the disappearance (as a percentage, such as any of about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 75%, 80%, 85%, 90%, 95%, or 100% relative to the amount of phytate remaining after passage through the rumen) as compared to a phytase polypeptide or a fragment thereof that does not comprise at least 82% sequence identity of the amino acid sequence set forth in SEQ ID NO:1. In some embodiments, the phytate is degraded in the presence of pepsin. In other embodiments, the phytate is degraded at a pH of about 1.5 to 3 (such as any of about 1.5, 1.6, 1.7, 1.8, 1.9, 2, 2.1, 2.2, 2.3, 2.4, 2.5, 2.6, 2.7, 2.8, 2.9, or 3).

The improvement in performance parameters may be in respect to a control in which the feed used does not comprise a phytase (such as any of the phytase or functional fragments thereof disclosed herein).

The terms “mineralization” or “mineralization” encompass mineral deposition or release of minerals. Minerals may be deposited or released from the body of the animal. Minerals may be released from the feed. Minerals may include any minerals necessary in an animal diet, and may include calcium, copper, sodium, phosphorus, iron and nitrogen.

Nutrient digestibility as used herein means the fraction of a nutrient that disappears from the gastro-intestinal tract or a specified segment of the gastro-intestinal tract, e.g. the small intestine. Nutrient digestibility may be measured as the difference between what is administered to the subject and what comes out in the faeces of the subject, or between what is administered to the subject and what remains in the digesta on a specified segment of the gastro intestinal tract, e.g., the ileum.

Nutrient digestibility as used herein may be measured by the difference between the intake of a nutrient and the excreted nutrient by means of the total collection of excreta during a period of time; or with the use of an inert marker that is not absorbed by the animal, and allows the researcher calculating the amount of nutrient that disappeared in the entire gastro-intestinal tract or a segment of the gastro-intestinal tract. Such an inert marker may be titanium dioxide, chromic oxide or acid insoluble ash. Digestibility may be expressed as a percentage of the nutrient in the feed, or as mass units of digestible nutrient per mass units of nutrient in the feed.

Nutrient digestibility as used herein encompasses phosphorus digestibility, starch digestibility, fat digestibility, protein digestibility, and amino acid digestibility. Digestible phosphorus (P) can be defined as ileal digestible P which is the proportion of total P intake absorbed at the end of the ileum by an animal or the fecal digestible P which is the proportion of total P intake that is not excreted in the feces.

The term “survival” as used herein means the number of subjects remaining alive. The term “improved survival” is another way of saying “reduced mortality”.

The term “carcass yield” as used herein means the amount of carcass as a proportion of the live body weight, after a commercial or experimental process of slaughter. The term carcass means the body of an animal that has been slaughtered for food, with the head, entrails, part of the limbs, and feathers or skin removed. The term meat yield as used herein means the amount of edible meat as a proportion of the live body weight, or the amount of a specified meat cut as a proportion of the live body weight.

An “increased weight gain” refers to an animal having increased body weight on being fed feed comprising a feed additive composition compared with an animal being fed a feed without said feed additive composition being present.

The terms “animal feed composition,” “feed”, “feedstuff,” and “fodder” are used interchangeably and can comprise one or more feed materials selected from the group comprising a) cereals, such as small grains (e.g., wheat, barley, rye, oats and combinations thereof) and/or large grains such as maize or sorghum; b) by products from cereals, such as corn gluten meal, Distillers Dried Grains with Solubles (DDGS) (particularly corn based Distillers Dried Grains with Solubles (cDDGS), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; and/or e) minerals and vitamins.

In further aspects, a feed can contain or comprise one or more cereal byproducts of a distillation process. As used herein, a “cereal byproduct of a distillation process” refers to a co-product or by-product from a fermentation of a feedstock or biomass (e.g., fermentation of grain or a grain mixture that produces a product alcohol). In some embodiments, a cereal byproduct of a distillation process may also refer to an animal feed product produced from a process of making a product alcohol (e.g., ethanol, butanol, isobutanol, etc.). In certain embodiments, the phytase or functional fragment thereof (such as any of those disclosed herein) is added to the cereal (such as, without limitation, grains, e.g., corn, wheat, rye, barley, oats, and mixtures thereof) during a saccharification and/or fermentation process. As used herein “saccharification” refers to the process of hydrolyzing polysaccharides and/or oligosaccharides, for example, alpha-1,4-glucosidic bonds of glycogen, or starch. Enzymes used during saccharification may include, without limitation, enzymes capable of hydrolyzing cellulosic or lignocellulosic materials as well (e.g., amylases, glucoamylases, proteases, etc.). “Fermentation” as used herein means a carbon source capable of being metabolized by a microorganism (e.g., yeast) for the production of a fermentative alcohol (e.g., ethanol). Suitable fermentable carbon sources include, but are not limited to, products of saccharification, for example, monosaccharides such as glucose or fructose; disaccharides such as lactose or sucrose; oligosaccharides; polysaccharides such as starch or cellulose; C5 sugars such as xylose and arabinose; one carbon substrates including methane; and mixtures thereof.

Suitably a premix as referred to herein may be a composition composed of microingredients such as vitamins, minerals, chemical preservatives, antibiotics, fermentation products, and other essential ingredients. Premixes are usually compositions suitable for blending into commercial rations.

As used herein the term “contacted” refers to the indirect or direct application of any of the engineered phytase polypeptides or fragments thereof (or composition comprising any of the engineered phytase polypeptides or fragments thereof) to a product (e.g. the feed). Examples of application methods which may be used, include, but are not limited to, treating the product in a material comprising the feed additive composition, direct application by mixing the feed additive composition with the product, spraying the feed additive composition onto the product surface or dipping the product into a preparation of the feed additive composition. In one embodiment, the feed additive composition of the present invention is preferably admixed with the product (e.g. feedstuff). Alternatively, the feed additive composition may be included in the emulsion or raw ingredients of a feedstuff. For some applications, it is important that the composition is made available on or to the surface of a product to be affected/treated. This allows the composition to impart a performance benefit.

Any of the engineered phytase polypeptides or fragments thereof described herein (or composition comprising such engineered phytase polypeptides or fragments thereof) may be applied to intersperse, coat and/or impregnate a product (e.g. a diet that contains no or substantially no inorganic phosphorus or feedstuff or raw ingredients of a feedstuff) with a controlled amount of said enzyme.

In another aspect, the feed additive composition can be homogenized to produce a powder. The powder may be mixed with other components known in the art. The powder, or mixture comprising the powder, may be forced through a die and the resulting strands are cut into suitable pellets of variable length.

Optionally, the pelleting step may include a steam treatment, or conditioning stage, prior to formation of the pellets. The mixture comprising the powder may be placed in a conditioner, e.g. a mixer with steam injection. The mixture is heated in the conditioner up to a specified temperature, such as from 60-100 °C, typical temperatures would be 70 °C, 80 °C, 85 °C, 90 °C or 95 °C. The residence time can be variable from seconds to minutes. It will be understood that any of the engineered phytase polypeptides or fragments thereof (or composition comprising any of the engineered phytase polypeptides or fragments thereof) described herein are suitable for addition to any appropriate feed material.

In other embodiments, the granule may be introduced into a feed pelleting process wherein the feed pretreatment process may be conducted between 70 °C and 95 °C for up to several minutes, such as between 85 °C and 95 °C.

In some embodiments, any of the engineered phytase polypeptides or fragments thereof can be present in the feed in the range of 1 ppb (parts per billion) to 10% (w/w) based on pure enzyme protein. In some embodiments, the engineered phytase polypeptides or fragments thereof are present in the feedstuff is in the range of 1-100 ppm (parts per million). A preferred dose can be 1-20 g of an engineered phytase polypeptide or fragment thereof per ton of feed product or feed composition or a final dose of 1-20 ppm engineered phytase polypeptide or fragment thereof in the final feed product.

Preferably, an engineered phytase polypeptide or fragment thereof is present in the feed should be at least about 50-10,000 FTU/kg corresponding to roughly 0.1 to 20 mg engineered phytase polypeptide or fragment thereof protein/kg.

Ranges can include, but are not limited to, any combination of the lower and upper ranges discussed above.

Formulations and/or preparations comprising any of the engineered phytase polypeptides or fragments thereof and compositions described herein may be made in any suitable way to ensure that the formulation comprises active phytase enzymes. Such formulations may be as a liquid, a dry powder or a granule which may be uncoated/unprotected or may involve the use of a thermoprotectant coating depending upon the processing conditions. As was noted above, the engineered phytase polypeptides and fragments thereof can be formulated inexpensively on a solid carrier without specific need for protective coatings and still maintain activity throughout the conditioning and pelleting process. A protective coating to provide additional thermostability when applied in a solid form can be beneficial for obtaining pelleting stability when required in certain regions where harsher conditions are used or if conditions warrant it, e.g., as in the case of super conditioning feed above 90 °C.

Feed additive composition described herein can be formulated to a dry powder or granules as described in WO2007/044968 (referred to as TPT granules) or WO1997/016076 or WO1992/012645 (each of which is incorporated herein by reference).

In one embodiment the feed additive composition may be formulated to a granule for feed compositions comprising: a core; an active agent (for example, a phytase, such as any of the engineered phytase polypeptides disclosed herein); and at least one coating, the active agent of the granule retaining at least 50% activity, at least 60% activity, at least 70% activity, at least 80% activity after conditions selected from one or more of a) a feed pelleting process, b) a steam-heated feed pretreatment process, c) storage, d) storage as an ingredient in an unpelleted mixture, and e) storage as an ingredient in a feed base mix or a feed premix comprising at least one compound selected from trace minerals, organic acids, reducing sugars, vitamins, choline chloride, and compounds which result in an acidic or a basic feed base mix or feed premix.

With regard to the granule at least one coating may comprise a moisture hydrating material that constitutes at least 55% w/w of the granule; and/or at least one coating may comprise two coatings. The two coatings may be a moisture hydrating coating and a moisture barrier coating. In some embodiments, the moisture hydrating coating may be between 25% and 60% w/w of the granule and the moisture barrier coating may be between 2% and 15% w/w of the granule. The moisture hydrating coating may be selected from inorganic salts, sucrose, starch, and maltodextrin and the moisture barrier coating may be selected from polymers, gums, whey and starch.

In other embodiments, the granule may be introduced into a feed pelleting process wherein the feed pretreatment process may be conducted between 70 °C and 95 °C for up to several minutes, such as between 85 °C and 95 °C.

The feed additive composition may be formulated to a granule for animal feed comprising: a core; an active agent, the active agent of the granule retaining at least 80% activity after storage and after a steam-heated pelleting process where the granule is an ingredient; a moisture barrier coating; and a moisture hydrating coating that is at least 25% w/w of the granule, the granule having a water activity of less than 0.5 prior to the steam-heated pelleting process.

The granule may have a moisture barrier coating selected from polymers and gums and the moisture hydrating material may be an inorganic salt. The moisture hydrating coating may be between 25% and 45% w/w of the granule and the moisture barrier coating may be between 2% and 10% w/w of the granule.

Alternatively, the composition is in a liquid formulation suitable for consumption preferably such liquid consumption contains one or more of the following: a buffer, salt, sorbitol and/or glycerol.

Also, the feed additive composition may be formulated by applying, e.g. spraying, the enzyme(s) onto a carrier substrate, such as ground wheat for example.

In one embodiment, the feed additive composition may be formulated as a premix. By way of example only the premix may comprise one or more feed components, such as one or more minerals and/or one or more vitamins.

In one embodiment a direct fed microbial (“DFM”) and/or an engineered phytase polypeptide or fragment thereof are formulated with at least one physiologically acceptable carrier selected from at least one of maltodextrin, limestone (calcium carbonate), cyclodextrin, wheat or a wheat component, sucrose, starch, Na₂SO₄, Talc, PVA, sorbitol, benzoate, sorbate, glycerol, sucrose, propylene glycol, 1,3-propane diol, glucose, parabens, sodium chloride, citrate, acetate, phosphate, calcium, metabisulfite, formate and mixtures thereof.

### EXAMPLES

Unless defined otherwise herein, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, N. Y. (1991) provide one of skill with a general dictionary of many of the terms used with this disclosure.

The disclosure is further defined in the following Examples. It should be understood that these Examples, while indicating certain embodiments, are given by way of illustration only. From the above discussion and the Examples, one skilled in the art can ascertain essential characteristics of this disclosure, and without departing from the spirit and scope thereof, can make various changes and modifications to adapt to various uses and conditions.

### Example 1: Materials and Methods

This Example describes the materials and methods used to produce the results in Examples 2 and 3. The study was carried out in accordance with the European Directive 2010/63 EU and the Dutch regulations for the care and use of animals in research. All experimental protocols and procedures were evaluated and approved by the Central Authority for Scientific Procedures on Animals (Centrale Commissie Dierproeven, Den Haag, The Netherlands) and by the Ethical Committee on Animal Experiments (Ethische Toetsing Dierproeven) of Schothorst Feed Research (Lelystad, The Netherlands).

Animals, diets and feeding: The experiment was carried out with 30 Holstein-Friesian dairy cows at the experimental farm of Schothorst Feed Research BV (Lelystad, The Netherlands). Cows were multiparous and at the start of the experiment averaged 34 kg milk/d, 158 days in milk, 690 kg BW and 3.3 lactations. Cows were kept in a free stall barn equipped with cubicles (1.10×2.5 m) bedded with chopped straw, rubber floors and had free access to water. Cows were monitored for health daily and any signs of clinical disease were recorded and treated appropriately.

Treatment diets comprised of a control diet (CON) formulated without supplemental Pi and two experimental diets based on the CON diet but supplemented with a commercial phytase (SEQ ID NO:26) at a targeted dose level of 2,000 or 5,000 phytase units (FTU) per kilogram of total ration on a DM basis. The phytase was a biosynthetic bacterial 6-phytase, PhyG (Danisco Animal Nutrition & Health, IFF Inc., The Netherlands), expressed in Trichoderma reesei.

Forage and concentrates were fed to the cows separately. Cows had access to individual Calan gates (American Calan, Northwood, NH) to measure the intake of the forage, and to an automated concentrate dispenser system (Hotraco, Hegelsom, The Netherlands) to supply and measure daily concentrate consumption. The forage component comprised a mixture of a constant grass silage to corn silage ratio (30:70 on a DM basis) and was offered ad libitum. The chemical composition of the forages is shown in Table 1.

TABLE 1
Chemical composition¹ of forages

| Item, g/kg dry matter | Grass | Corn |
|---|---|---|
| (unless otherwise stated) | silage | silage |
| Dry matter g/kg | 604 | 332 |
| Ash | 94 | 42 |
| Crude protein | 157 | 72 |
| Crude fat | 37 | 31 |
| Neutral detergent fibre | 511 | 349 |
| Acid detergent fibre | 295 | 211 |
| Acid detergent lignin | 28 | 23 |
| Starch | n.d. | 349 |
| Sugar | 105 | 14 |
| Calcium² | 4.4 | 1.7 |
| Phosphorus (P)² | 3.0 | 1.8 |
| Phytate-P³ | 0.0 | 0.0 |

¹Analyzed by Eurofins Agro NL (Wageningen, The Netherlands) and based on near infrared spectroscopy.
²The content of Ca was determined based on atomic absorption spectroscopy according to ISO 6869 (ISO, 2000), and P content was determined based on the colorimetric method according to ISO 6491 (ISO, 1998).
³Analyzed by Danisco Animal Nutrition Research Centre (Brabrand, Denmark) using a modified version of the HPLC method described by Skoglund et al. (1998).
n.d. = not determined.

Forages were mixed and provided to cows twice daily (at approximately 0700 and 1400 h) via an automated feeding system (Triomatic HP 2 300 hanging feeding robot) equipped with the Triomatic T40 feed kitchen with storage bunkers (Trioliet Feeding Technology, Oldenzaal, The Netherlands). Feed refusals were removed and weighed daily. The concentrates were manufactured by ABZ Diervoeding (Leusden, The Netherlands) and pelleted (exit temperature between 70 and 75 °C). The concentrates were supplied three times daily (at approximately 0500, 1230 and 1830 h) via the automated dispenser system into separate feeding buckets, in amounts that were individualized per cow based on fat- and protein-corrected milk (FPCM)-yield. Before producing the concentrates, ingredients were sampled for the analysis of PP and total P content. Afterwards, concentrates were optimized to contain a minimum of 2.3 g/kg of PP and a maximum of 3.1 g/kg of total P (equal to 2.8 g/kg DM on a total ration basis). Concentrates were formulated to contain phytate-rich ingredients with low ruminal degradability such as formaldehyde treated rapeseed meal and hydrothermal pressure-treated sunflower seed meal. For the estimation of fecal excretion, the external marker titanium dioxide (TiO₂) was added to the concentrates, at a level of 7 g/kg (as is). The ingredients and chemical composition of the concentrates is given in Table 2, whilst the calculated chemical composition of the total rations is presented in Table 3. The total rations were formulated to meet nutrient requirements as recommended by the Dutch system (CVB, 2018) except for P that formulated at a level of 2.8 g/kg DM in the total ration and aimed to supply 90% of the recommended P requirement according to COMV (2005).

TABLE 2
Ingredients, chemical composition, and
phytase activity of the concentrates

| | Concentrates¹ | | |
|---|---|---|---|
| Item | CON | PhyG2,000 | PhyG5,000 |

Ingredients, g/kg as fed

| | | | |
|---|---|---|---|
| Beetpulp | 427 | 427 | 427 |
| Sunflower meal | 119 | 119 | 119 |
| Rape seed meal | 99.3 | 99.3 | 99.3 |
| Wheat gluten meal | 77.7 | 77.7 | 77.7 |
| Corn | 68.9 | 68.9 | 68.9 |
| Oat hulls | 66.5 | 66.5 | 66.5 |
| Molasses beet | 49.7 | 49.6 | 49.6 |
| Corn gluten meal | 19.9 | 19.9 | 19.9 |
| Palm oil | 18.1 | 18.1 | 18.1 |
| Potato protein | 13.5 | 13.5 | 13.5 |
| Mineral premix² | 12.4 | 12.4 | 12.4 |
| Urea | 9.93 | 9.93 | 9.93 |
| Rumen-protected lysine | 3.97 | 3.97 | 3.97 |
| Magnesium oxide | 3.08 | 3.08 | 3.08 |
| Limestone | 1.99 | 1.99 | 1.99 |
| Salt | 1.79 | 1.79 | 1.79 |
| Titanium dioxide | 7.00 | 7.00 | 7.00 |

Chemical composition, g/kg dry matter (unless otherwise stated)

| | | | |
|---|---|---|---|
| Dry matter, g/kg | 901 | 901 | 902 |
| Ash | 81.0 | 82.1 | 82.0 |
| Crude protein | 269 | 269 | 268 |
| Crude fat | 46.6 | 46.6 | 46.6 |
| Neutral detergent fibre | 240 | 245 | 248 |
| Starch | 82.1 | 81.0 | 78.7 |
| Sugar | 155 | 161 | 159 |
| Calcium | 7.99 | 8.21 | 7.87 |
| Phosphorus (P) | 3.66 | 3.66 | 3.66 |
| Phytate-P | 2.33 | 2.11 | 2.00 |

Phytase, FTU/kg dry matter

| | | | |
|---|---|---|---|
| Total | 431 | 6,181 | 21,725 |
| Intrinsic | 431 | 431 | 431 |
| Exogenous | 0 | 5,750 | 21,294 |

¹CON, control; PhyG2,000, containing PhyG phytase at 2,000 FTU/kg total ration on DM basis; PhyG5,000, containing PhyG phytase at 5,000 FTU/kg total ration on DM basis.
²Chemical composition according to manufacturer in g/kg: 132 Ca, 1 P, 140 Mg, 75 Na, 116 Cl, 3 K, 0.9 S, 400 mg of Cu, 600 mg of Zn, 1600 mg of Mn 1,600 mg, 25 mg of Co, 110 mg of I, 30 mg of Se, 750,000 IU of vitamin A, 200,000 IU of vitamin D3, 1,500 IU of vitamin E.

TABLE 3
Chemical composition¹, feed values and phytase
activity of total dietary treatments

Dietary treatment²

| | | | |
|---|---|---|---|
| Item | CON | PhyG2,000 | PhyG5,000 |

Chemical composition, g/kg dry matter

| | | | |
|---|---|---|---|
| Dry matter, g/kg | 474 | 470 | 469 |
| Ash | 65 | 65 | 66 |
| Crude protein | 155 | 152 | 150 |
| Crude fat | 35 | 35 | 35 |
| Neutral detergent fibre | 358 | 360 | 357 |
| Acid detergent fibre | 211 | 212 | 214 |
| Acid detergent lignin | 24 | 24 | 26 |
| Starch | 178 | 181 | 181 |
| Sugar | 73 | 72 | 73 |
| Calcium | 4.4 | 4.3 | 4.2 |
| Phosphorus (P) | 2.6 | 2.6 | 2.6 |
| Phytate-P | 0.7 | 0.7 | 0.6 |

Feed value³

| | | | |
|---|---|---|---|
| VEM/kg DM | 980 | 978 | 978 |
| DVE, g/kg DM | 87 | 86 | 86 |

Phytase activity, FTU/kg dry matter

| | | | |
|---|---|---|---|
| Total phytase | 136 | 1,949 | 6,532 |
| Exogenous phytase | 0 | 1,813 | 6,403 |

¹Calculated from analyzed values presented in Tables 1 and 2, accounting for feed intake.
²CON, control; PhyG2,000, containing PhyG phytase at 2,000 FTU/kg total ration on DM basis; PhyG5,000, containing PhyG phytase at 5,000 FTU/kg total ration on DM basis.
³Feed value of diets estimated according to the Dutch evaluation system (CVB, 2018), where VEM is net energy for lactation, DVE is metabolizable protein.

An important prerequisite to determining whether an exogenous phytase will improve P digestibility is that the animals should be fed below the P requirement. To achieve this, the total rations were formulated to contain a low P content (without inorganic phosphate in the concentrate) that represented approximately 90% of the total P requirement according to the Dutch guideline (COMV, 2005). The total rations contained P at 2.6 g/kg DM in all treatments. A second important factor was to ensure that the diets were representative of commercial diets and that they contained rumen by-pass phytate-rich ingredients such as sunflower meal and rapeseed meal. The employed phytase dose levels (2,000 and 5,000 FTU/kg DM) were selected based on the dose levels of other exogenous microbial 6-phytases that have been reported in the literature to improve P digestibility in dairy cows (Brask-Pedersen et al., 2013; Winter et al. 2015) and consideration of the economic feasibility of including PhyG phytase in dairy cow diets.

Experimental design: The experiment was carried out as a randomized block design with three dietary treatments and 10 blocks (replicates) per treatment. The experiment comprised an 18-d pre-period for the collection of data to facilitate the allocation of cows to the treatments, followed by a 19-d experimental period comprising a 14-d diet adaptation phase, as is recommended for digestibility trials (GfE, 1991), and 5 days of feces collection. During the pre-period, all cows were fed ad libitum with a mixture of grass silage and corn silage in the same proportions as mentioned above and were supplemented with concentrates (as described above but without supplemental phytase) based on FPCM production.

### Sampling, Measurements, and Chemical Analyses:

Milk: Cows were milked twice daily starting at 0400 and 1530 h in a double-12 rapid exit milking parlor. Milk yield was individually recorded at each milking using calibrated electronic milk meters (DemaTron 70, GEA, Düsseldorf, Germany). Milk samples were collected per week from each cow on Monday evening, Tuesday morning, Wednesday evening and Thursday morning. Milk samples were preserved with a solution containing sodium azide and bronopol (0.3 mL of solution added per 50 mL of milk) and analyzed for fat, protein, lactose, urea, and somatic cell count by an accredited Dutch laboratory for monitoring milk quality (Qlip, Zutphen, The Netherlands), using Fourier transform infrared spectroscopy (MilkoScan FT6000/7, Foss Electric, Hillerød, Denmark). Two extra milk samples (50 mL) were taken per cow (morning and evening) during the last week of the experiment, pooled 1:1 (v:v) and analyzed for P content according to the spectrometric method ISO6491 (ISO, 1998).

Feed: The daily amounts of basal diet (forages) and concentrates offered and refused were individually recorded. The daily feed intake was calculated as the difference between the offered and refused amount for both basal diet and concentrates. Representative weekly samples of forages were collected, pooled per forage type by mixing equal amounts [on a fresh matter (FM) basis], and sent to the certificated laboratory Eurofins Agro NL (Wageningen, the Netherlands) for chemical analysis based on near infrared spectroscopy (NIRS). The concentrates were produced in one batch each, immediately sampled and analyzed. The chemical composition of these forages and concentrate samples was used to calculate the chemical composition of the total rations. During the final week of the Exp. and two days prior to the start of fecal sampling, additional samples of the forages and concentrates were collected for the determination of the apparent total tract digestibility (ATTD) of chemical constituents. Forages were sampled daily and concentrates every two days. Samples were stored at −20 °C until later analysis. At the end of the experiment, forages were thawed at room temperature, pooled per type of forage by mixing equal amounts on a FM basis, freeze-dried for approximately 96 h in a Zirbus sublimator 3-4-5/20 (Zirbus Technology Benelux B. V., Tiel, Netherlands) and ground to pass through a 1-mm screen using a Retsch ZM200 grinder (Retsch Benelux, Aartselaar, Belgium). The forage and concentrate samples were analyzed by Schothorst Feed Research (Lelystad, the Netherlands). The DM content was determined by drying at 103 °C to constant weight according to method ISO 6496 (ISO, 1998). Crude ash was determined gravimetrically after ashing the samples in a muffler furnace for 3 h at 550 °C, according to method ISO 5984 (ISO, 2002). The N content was determined by the Dumas method using a macro determinator (LECO CM928 MLC, LECO, Michigan, USA) according to method ISO 16634 (ISO, 2016), and the CP content was calculated as N×6.25. The starch content (except in grass silage) was determined by the amylo-glucosidase method according to the procedures of Englyst et al. (1992), and sugar content was determined according to the Luff-Schoorl method. Crude fat (CFat) was determined by ether extraction after acid hydrolysis, according to method ISO 11085 (ISO, 2015). The NDF content was exclusive of residual ash and a heat-stable α-amylase was added during NDF extraction, according to ISO 16472 (ISO, 2006). The ADF content was exclusive of ash and determined according to ISO 13906 (ISO, 2008). The P content was determined based on the colorimetric method according to ISO 6491 (ISO, 1998) and contents of Ca and TiO₂ were determined based on atomic absorption spectroscopy according to ISO 6869 (ISO, 2000). The content of PP in forages and concentrates was analyzed at Danisco Animal Nutrition Research Centre (Brabrand, Denmark) using the HPLC method described by Christensen et al. (2020) modified from Skoglund et al. (1998). Modifications to the analytical procedure were that the extraction of IP6 from the feces samples was carried out at a concentration of 0.20 g/mL using 1.0M HCl as solvent. The phytase activity in concentrate samples was analyzed by Danisco Animal Nutrition Research Centre (Brabrand, Denmark) according to a modified version of the 2000.12 AOAC method (Engelen et al., 2001). For this, one FTU was defined as the quantity of enzyme that released 1 μmol of inorganic orthophosphate from a 0.0051 mol/L sodium phytate substrate per minute at pH 5.5 at 37 °C. Feces: Fecal grab samples (~500 g of fecal matter) were collected from all animals twice daily during the last five days of the Exp. Fecal sampling commenced at 0900 and 1300 h on d 1, 3 and 5; and at 1100 and 1430 h on d 2 and 4. This sampling pattern was applied to account for diurnal and day-to-day variations in marker excretion (Glindemann et al., 2009). Samples were immediately frozen at −20 °C and stored until later analysis. At the end of the experiment, fecal samples were thawed at room temperature, pooled per cow on an equal-weight (of FM) basis, freeze-dried for approximately 96 h in a Zirbus sublimator 3-4-5/20 (Zirbus Technology Benelux B. V., Tiel, Netherlands), and ground to pass a 2-mm screen using a Retsch ZM200 grinder (Retsch Benelux, Aartselaar, Belgium). The content of moisture, CP (N×6.25), NDF, starch, P, Ca, PP and TiO₂ was analyzed using the aforementioned methods.

Blood: Blood samples were taken from each cow from the coccygeal vein on day 4 of the collection period at approximately 1200 h. Samples were analyzed for total (free) P, without a destruction step, according to method ISO 6491 (ISO, 1998). The serum cross-linked C-telopeptide of type I collagen (CTX), a marker for bone turnover, was analyzed according to a CTX-I ELISA method (IDS Plc., Tyne & Wear, UK).

BW and body condition score (BCS): Individual BW and BCS were recorded twice daily directly after each milking. The BW was recorded via automatic weighing scale and the BCS was recorded using an automatic BCS system (DeLaval, Kansas City, MO). Body condition was scored based on the 1 to 5 scale method of Edmonson et al. (1989), where 1=very thin and 5=obese.

Sample Size, calculations and statistical analysis: The sample size calculation was based on a two-sided test with a confidence level of 95% and a power of 0.80 to detect a statistically significant difference in ATTD of P in the phytase supplemented treatments compared to CON. The expected effect size was based on published data concerning the variance in P digestibility in lactating dairy cows (Valk et al., 2002; Wu et al., 2003; Kincaid et al., 2005; Knowlton et al. 2007). The chemical composition of the total rations was calculated based on the chemical composition and intakes of both forages and concentrates. The fecal excretion of DM was calculated for each cow from the daily TiO₂ administration (g/animal) divided by the TiO₂ concentration (g/kg DM) in feces. For this, a fecal recovery of TiO₂ of 100% (Glindemann et al., 2009) was assumed. The fecal excretion of CP, starch, NDF, P, Ca and PP was calculated as DM fecal excretion multiplied by the concentration of the respective chemical component in the feces. The ATTD of DM, CP, starch, NDF, P, Ca and PP was computed as ATTD (%)=[(intake—feces excretion)/intake]. For this calculation, the intake and feces excretion of each chemical component was in kg/d on a DM basis. The yield of FPCM (kg/d) was calculated on a 4% fat and 3.3% protein basis. The feed efficiency was calculated as FPCM divided by DMI both expressed in kg. Data on SCC were log transformed to obtain a normal distribution before statistical analysis. All data were averaged per cow and week for the statistical analysis.

All statistical analyses were performed using Genstat 18th edition (VSN International, Hemel Hempstead, UK). Data were analyzed by ANOVA to identify treatment effects. Treatment means comparisons were carried out using the Tukey test. Data are presented as least squares means and associated pooled SEM values. The statistical analyses for all variables (except P in milk and blood, and ATTD) was carried out using the data of the pre-period as a covariate, using the following model:

Yijk=μ+Blocki+Covj+Trtk+εijk

where, Yijk is the response variable, u is the overall mean, Blocki is the effect of block (i=1-10), Covj is the covariate (response during pre-period), Trtk is the effect of dietary treatment (k=1-3) and sijkl is the residual error.

For the statistical analysis of P content in milk and blood, and ATTD, the same model was used but without the pre-period as a covariate. In addition, the effect of phytase dose level on nutrient intake ATTD and fecal excretion of nutrients during the fecal collection period was analyzed by polynomial contrasts to determine the linear and quadratic response to increasing phytase dose, with consideration of uneven distribution between phytase dose levels. Statistical significance was declared at P<0.05. 0.05<P<0.1 was considered a tendency.

Chemical composition of diets: In general, the chemical composition of the concentrates (Table 2) and total rations (Table 3) was similar among treatments. For the total rations, although the content of P was slightly lower than the targeted value (2.8 g/kg DM), the content among treatments was similar (2.6 g/kg DM for all). The PP level of the total rations was also slightly lower than formulated (−0.2 g/kg DM) but again was similar among treatments (0.6 to 0.7 g/kg DM). Phytase activity in the CON concentrate was low (431 FTU/kg, DM basis; Table 2). Because no phytase was added to the CON concentrate, this activity was considered to have been from the intrinsic phytase of the feed ingredients in the concentrate. After accounting for this native phytase activity in the concentrates, the exogenous phytase activities in the total rations were calculated as 1,813 and 6,403 FTU/kg DM in PhyG2,000 and PhyG5,000, respectively (Table 3). Thus, the administered exogenous phytase to the total diets (in FTU/kg DM) and the differences between dietary phytase-treatments were consistent with the target values.

### Example 2: Feed Intake, Milk Yield and Composition, and Blood Analysis

The effect of PhyG supplementation on BW, feed intake, feed efficiency, milk yield and composition, and blood analytes are presented in Table 4.

TABLE 4
Effect of phytase supplementation on BW, dry matter intake, feed
efficiency, milk production and composition (during the 19-d
experimental period) and on blood analytes (measured on d 18)

| | Dietary treatment¹ | | | | P- |
|---|---|---|---|---|---|
| Item | CON | PhyG2,000 | PhyG5,000 | SEM | value |

Dry matter intake, kg/d

| | | | | | |
|---|---|---|---|---|---|
| Forage | 17.8 | 17.6 | 18.7 | 0.46 | 0.22 |
| Concentrate | 8.2 | 8.0 | 8.0 | 0.21 | 0.76 |
| Total ration | 26.0 | 25.4 | 26.7 | 0.40 | 0.10 |

Milk production

| | | | | | |
|---|---|---|---|---|---|
| Milk, kg/d | 33.8 | 33.1 | 33.7 | 0.36 | 0.31 |
| FPCM³, kg/d | 36.1 | 35.5 | 36.0 | 0.45 | 0.55 |
| Fat, g/d | 1499 | 1471 | 1484 | 28.1 | 0.79 |
| Protein, g/d | 1228 | 1210 | 1243 | 15.1 | 0.33 |
| Lactose, g/d | 1522 | 1487 | 1520 | 17.6 | 0.31 |

Milk composition

| | | | | | |
|---|---|---|---|---|---|
| Fat, % | 4.45 | 4.47 | 4.48 | 0.08 | 0.97 |
| Protein, % | 3.68^{B} | 3.69^{AB} | 3.72^{A} | 0.014 | 0.08 |
| Lactose, % | 4.49 | 4.50 | 4.49 | 0.013 | 0.97 |
| Phosphorus, g/L | 0.98 | 0.96 | 0.98 | 0.03 | 0.86 |
| Urea, mg/dL | 22.4 | 21.7 | 22.5 | 0.613 | 0.61 |
| Somatic cell count, Log₁₀, cells/ml | 1.7 | 1.7 | 1.6 | 0.049 | 0.32 |
| BW, kg | 692 | 688 | 689 | 3.13 | 0.61 |
| Body condition score | 3.11 | 3.08 | 3.07 | 0.02 | 0.43 |
| Feed efficiency³ | 1.37 | 1.39 | 1.34 | 0.02 | 0.19 |

Blood analytes

| | | | | | |
|---|---|---|---|---|---|
| Phosphorus, mg/L | 46.7 | 48.4 | 50.1 | 2.20 | 0.56 |
| CTX, ng/mL⁴ | 1.95 | 1.53 | 1.66 | 0.24 | 0.46 |

¹CON, control; PhyG2,000, containing PhyG phytase at 2,000 FTU/kg total ration on DM basis; PhyG5,000, containing PhyG phytase at 5,000 FTU/kg total ration on DM basis.
²Fat and protein corrected milk production (FPCM)
³Calculated as FPCM/DMI, both in kg
⁴CTX, Serum cross-linked C-telopeptide of type I collagen.
^{A,B}Means within a row bearing different upper case capital letters are different at a statistical tendency level of P < 0.1.

Dietary treatment had no effect on milk production or FPCM, that averaged 33.5 and 35.9 kg/d, respectively. Similarly, fat and lactose contents were unaffected by treatment. Milk protein content tended to be affected by dietary treatment (P=0.08); cows fed PhyG5,000 tended to have a higher milk protein content than cows fed CON (3.72 vs. 3.68%, respectively). The P content of milk and of blood, and bone metabolism as indicated by CTX concentration, were unaffected by treatment. There was also no effect of treatment on BW, BCS, or feed efficiency. The DMI was similar between treatments and averaged 26.0 kg/d.

The observed tendency towards a higher content of milk protein in the highest phytase dose treatment compared to the control is consistent with the increased CP digestibility discussed below and suggests there was a higher availability of protein in the PhyG5,000 diet, due to the activity of the phytase. Previous studies have not observed an effect of exogenous phytase on milk protein content in dairy cows. Without being bound to theory, it is hypothesized that the higher CP digestibility led to increased AA absorption and that this increased the availability of AA for milk protein synthesis; it is known that milk protein synthesis is highly reliant on the availability of AA, particularly of methionine, lysine and histidine (Kim and Lee, 2021).

### Example 3: Nutrient Intake, Excretion, and ATTD

The effect of treatment on nutrient intake, ATTD and fecal excretion during the last 5 days of the Exp. is presented in Table 5. There was no effect of treatment on total DMI during this period. Because of this, and the similar chemical composition of the total rations, there was also no effect of treatment on the intake of CP, starch, NDF, total P or Ca. However, intake of PP tended to be lower (P=0.06) in Phy5,000 (16.5 g/d) than CON (18.7 g/d), but was similar in Phy2,000 (17.0 g/d).

TABLE 5
Effect of phytase supplementation on nutrient intake, fecal excretion, and apparent
total tract digestibility (ATTD) during the 5-d fecal collection period

| | Dietary treatment1 | | | | | Polynomial contrasts | |
|---|---|---|---|---|---|---|---|
| | | PhyG | PhyG | | ANOVA | ‘linear’ | ‘quadratic’ |
| Item | CON | 2,000 | 5,000 | SEM | P-value | P-value | P-value |
| Intake | | | | | | | |
| Dry matter, kg/d | 27.1 | 26.2 | 27.6 | 0.49 | 0.15 | 0.64 | 0.24 |
| Crude protein, kg/d | 3.97 | 3.83 | 4.00 | 0.70 | 0.26 | 0.86 | 0.36 |
| Starch, kg/d | 5.04 | 4.84 | 5.13 | 0.12 | 0.24 | 0.59 | 0.21 |
| Neutral detergent fibre, kg/d | 9.72 | 9.41 | 10.0 | 0.20 | 0.16 | 0.44 | 0.23 |
| Phosphorus (P), g/d | 69.9 | 67.3 | 70.6 | 1.16 | 0.15 | 0.80 | 0.29 |
| Calcium, g/d | 116 | 114 | 115 | 2.09 | 0.80 | 0.72 | 0.66 |
| Phytate-P, g/d | 18.7^{B} | 17.0^{AB} | 16.5^{A} | 0.63 | 0.06 | 0.24 | 0.40 |
| Fecal excretion | | | | | | | |
| Dry matter, kg/d | 8.19^{B} | 7.43^{A} | 7.58^{AB} | 0.22 | 0.06 | 0.16 | 0.25 |
| Crude protein, kg/d | 1.36^{b} | 1.21^{a} | 1.22^{a} | 0.03 | 0.01 | 0.08 | 0.21 |
| Starch, g/d | 126 | 120 | 105 | 8.30 | 0.23 | 0.15 | 0.58 |
| Neutral detergent fibre, kg/d | 4.18^{B} | 3.74^{A} | 4.02^{AB} | 0.13 | 0.08 | 0.40 | 0.20 |
| Phosphorus (P), g/d | 37.4^{b} | 33.6^{a} | 32.5^{a} | 1.11 | 0.02 | 0.01 | 0.34 |
| Calcium, g/d | 87.5^{b} | 86.5^{b} | 77.5^{a} | 2.62 | 0.04 | 0.06 | 0.93 |
| Phytate-P, g/d | 1.35^{a} | 0.87^{b} | 0.46^{c} | 0.095 | <0.001 | <0.001 | 0.04 |
| ATTD, % | | | | | | | |
| Dry matter | 69.9^{A} | 71.6^{AB} | 72.7^{B} | 0.76 | 0.06 | 0.01 | 0.49 |
| Crude protein | 65.7^{a} | 68.4^{b} | 69.5^{b} | 0.70 | <0.01 | <0.01 | 0.20 |
| Starch | 97.5 | 97.5 | 98.0 | 0.16 | 0.13 | 0.10 | 0.81 |
| Neutral detergent fibre | 57.1 | 60.2 | 59.9 | 1.29 | 0.21 | 0.11 | 0.20 |
| Phosphorus (P) | 46.3^{a} | 49.7^{ab} | 54.1^{b} | 1.52 | <0.01 | <0.01 | 0.99 |
| Calcium | 24.1^{A} | 23.3^{A} | 33.3^{B} | 2.85 | 0.05 | 0.02 | 0.37 |
| Phytate-P | 92.6^{a} | 95.1^{b} | 97.2^{c} | 0.45 | <0.001 | <0.001 | 0.13 |

CON, control; PhyG2,000, containing PhyG phytase at 2,000 FTU/kg; PhyG5,000, containing PhyG phytase at 5,000 FTU/kg.
^{A, B}Means within a row bearing different superscript upper case letters are different at a statistical tendency level of P < 0.1.
^{a, b, c}Means bearing different superscript lower case letters within a row are significantly different at P < 0.05.

Dietary treatment affected the fecal excretion of CP (P=0.01), total P (P=0.02), PP (P<0.001) and Ca (P=0.04). Fecal excretion of CP, total P and PP was reduced in cows fed PhyG2,000 and PhyG5,000 compared to CON (by 10.3%, 13.1% and 65.9%, respectively, in PhyG5,000 vs. CON). Fecal excretion of total P decreased linearly (P=0.01) whilst that of PP decreased both linearly and quadratically with increasing PhyG dose level (P<0.001 and P<0.05, respectively) and that of CP tended to decrease linearly (P=0.08). Excretion of Ca was reduced (by 11.4%) in PhyG5,000 but not in PhyG2,000 compared to CON, and tended (P=0.06) to reduce linearly with increasing PhyG dose level. Dietary treatment also tended to affect the fecal excretion of DM (P=0.06) and NDF (P=0.08), without a linear or quadratic dose-response effect.

The ATTD of CP, total P, and PP were all affected by treatment (P<0.01, P<0.01 and P<0.001, respectively) and tended to affect ATTD of DM (P=0.06) and Ca (P=0.05). The ATTD CP was higher for cows fed PhyG2,000 and PhyG5,000 than CON (+2.7 and +3.8% points, respectively; P<0.05) whilst ATTD of P was higher in cows fed PhyG5,000 compared to CON (by 7.8% points; P<0.05) and ATTD of Ca tended to be higher in cows fed PhyG5,000 compared to CON (by 9.2% points). There were positive linear relationships between PhyG dose level and ATTD of DM, CP, P, Ca and PP (P=0.01, P<0.001, P<0.001, P<0.05 and P<0.001, respectively). The increase in ATTD of PP between 0 and 5,000 FTU/kg was 4.6% points.

Among the treatments, cows exhibited a similar DMI, and because the chemical composition was similar between diets, the intake of all chemical components among treatments was also similar, except for PP. The PP intake was 12% lower with PhyG5,000, compared to the control diet. This appears to have resulted from the combination of a lower PP content of the concentrate in this treatment (2.33 vs. 2.00 g/kg DM in CON and PhyG5,000, respectively, with the same basal diet), together with a lower-than-formulated concentrate-to-forage intake ratio in PhyG5,000 (30% in PhyG5,000, 32% in CON on a DM basis compared to the formulated ratio of 35%). A reduced PP intake could, in theory, have contributed to the observed differences in nutrient digestibility and excretion in the PhyG5,000 treatment compared to the control diet. However, the reduction in excretion of PP in this diet was much greater than the reduction in PP intake (PP excretion reduction was 66% greater than CON in PhyG5,000) suggesting, without being bound to theory, that the observed increases in nutrient digestibility and reductions in nutrient excretion in PhyG5,000 were due to the activity of the added phytase and not the differences in PP intake.

This experiment also showed a clear effect of phytase supplementation on the ATTD of P and PP. This adds further weight to the hypothesis that, contrary to past beliefs, the PP of feeds may not be fully utilized by dairy cows. This is particularly the case when phytate-rich and rumen-protected ingredients are included. Without being bound to theory, it is in such diets that exogenous phytase is expected to have most benefit. In the present study, cows exhibited ATTD of PP increases (above CON) of 2.5 and 4.6% points with PhyG dosed at 1,813 and 6,403 FTU/kg DM total ration, respectively (based on analyzed phytase activity).

In conclusion, the ATTD of P, PP and Ca were improved by dietary supplementation of low P diets with a biosynthetic bacterial 6-phytase in a linear dose-dependent manner. In addition, CP digestibility was increased and milk protein content tended to be increased by the biosynthetic phytase. This study has clearly shown that supplementation of the novel phytase to dairy cow diets offers potential as an approach for improving P and protein utilization. In this way, phytase supplementation offers an important approach to optimizing nutrient balance and reducing environmental P and N pollution from dairy farms.

### Example 4: Materials and Methods

This Example describes the materials and methods used to produce the results in Examples 5 and 6. The study was carried out at the Educational and Research Centre of Animal Husbandry, Hofgut Neumühle, Germany, in January 2023, as a joined project with University of Applied Science Bingen. All protocols and experimental procedures were reviewed and approved by the Department for Animal Welfare Affairs (Landesuntersuchungsamt Rheinland-Pfalz (LUA-RLP), Koblenz, Germany) and carried out in accordance with the German Animal Welfare Act.

Animals, diets and feeding: The experiment was carried out with 47 Holstein-Friesian dairy cows. Cows were multiparous and at the start of the experiment averaged 44.1±6.0 kg milk/d, 653±63 kg BW and 3.1 lactations. Cows were kept in a loose housing system with free access to cubicles (1.23×2.5/2.75 m), concrete floors and had free access to water. The barn was equipped with 30 weighing feeding troughs. Cows were monitored for health daily and any signs of clinical disease were recorded and treated appropriately.

Treatment diets comprised of a control diet (CON) formulated without supplemental phytase and two experimental diets based on the CON diet but supplemented with PhyG at a targeted dose level of 2,000 or 5,000 phytase units (FTU) per kilogram of total ration on a dry matter (DM) basis. The phytase was the same phytase used in Example 1.

Treatment diets were provided to cows as a Total Mixed Ration (TMR) containing a forage component comprised of a mixture of grass silage, corn silage and pulp silage and a concentrate component formulated to contain phytate-rich ingredients with low ruminal degradability such as formaldehyde-treated rapeseed meal and hydrothermal pressure-treated sunflower seed meal.

The chemical composition of the forages is shown in Table 6

TABLE 6
Analyzed chemical composition¹ of the forages

| Item, g/kg dry matter | Grass | Corn | Pulp |
|---|---|---|---|
| (unless otherwise stated) | silage | silage | silage |
| Dry matter g/kg | 248 | 251 | 259 |
| Ash | 89 | 38 | 73 |
| Crude protein | 147 | 68 | 96 |
| Crude fat | 43 | 32 | 6 |
| Neutral detergent fibre | 586 | 437 | 524 |
| Starch | 9 | 271 | 237 |
| Sugar | n.d. | n.d. | 17 |
| Calcium | 4.3 | 2.5 | 10.7 |
| Phosphorus (P) | 3.5 | 2.3 | 0.93 |
| Phytate-P | 0 | 0 | 0 |

n.d. = not determined.

The concentrates were manufactured by ABZ Diervoeding (Leusden, the Netherlands) in mash form. Before producing the concentrates, ingredients were sampled for the analysis of phytate-P and total P. Afterwards, concentrates were optimized to contain a minimum of 2.3 g/kg of phytate-P and a maximum of 3.1 g/kg total P (equal to 2.8 g/kg DM on a total ration basis). The ingredient and chemical composition and analyzed phytase activity of the concentrates is shown in Table 7.

TABLE 7
Ingredients, chemical composition, and
phytase activity of the concentrates

| | Concentrates¹ | | |
|---|---|---|---|
| Item | CON | PhyG2,000 | PhyG5,000 |

Ingredients, g/kg as fed

| | | | |
|---|---|---|---|
| Beetpulp | 390 | 390 | 390 |
| Sunflower meal | 135 | 135 | 135 |
| Rapeseed meal | 120 | 120 | 120 |
| Wheat gluten meal | 50 | 50 | 50 |
| Corn | 70 | 70 | 70 |
| Oat hulls | 70 | 70 | 70 |
| Molasses beet | 50 | 50 | 50 |
| Corn gluten meal | 60 | 60 | 60 |
| Palm oil | 18 | 18 | 18 |
| Potato protein | 10 | 10 | 10 |
| Urea | 10 | 10 | 10 |
| Salt | 4 | 4 | 4 |
| Limestone | 4 | 4 | 4 |
| Mineral premix² | 3 | 3 | 3 |
| Magnesium oxide | 3 | 3 | 3 |
| Rumen protected lysine | 3 | 3 | 3 |

Chemical composition, g/kg dry matter (unless otherwise stated)

| | | | |
|---|---|---|---|
| Dry matter, g/kg | 895 | 893 | 900 |
| Ash | 79 | 76 | 78 |
| Crude protein | 316 | 289 | 299 |
| Crude fat | 45 | 41 | 43 |
| Neutral detergent fibre | 306 | 313 | 272 |
| Starch | 156 | 164 | 167 |
| Sugar | 123 | 125 | 122 |
| Calcium | 7.0 | 6.9 | 6.6 |
| Phosphorus (P) | 4.13 | 3.70 | 3.70 |
| Phytate-P | 2.41 | 2.32 | 2.32 |

Phytase, FTU/kg dry matter

| | | | |
|---|---|---|---|
| Total | 508 | 6345 | 19633 |
| Intrinsic | 508 | | |
| Exogenous | — | 5837 | 19125 |

¹CON, control; PhyG2,000, containing PhyG phytase at 2,000 FTU/kg total ration on DM basis; PhyG5,000, containing PhyG phytase at 5,000 FTU/kg total ration on DM basis.
²Chemical composition according to manufacturer in g/kg: 132 Ca. 1 P. 140 Mg, 75 Na, 116 Cl, 3 K, 0.9 S, 400 mg of Cu, 600 mg of Zn, 1600 mg of Mn, 25 mg of Co, 100 mg of I, 30 mg of Se, 750,000 IU of vitamin A, 200,000 IU of vitamin D3, 1500 IU of vitamin E.

On a dry matter basis, TMR contains 36.0% of concentrate, 25.3% of corn silage, 19.9% of grass silage, 18.8% of pulp silage. For the estimation of fecal excretion and total tract digestibility, the external marker titanium dioxide (TiO₂) was added on top of to the concentrates, at a level of 0.7% in concentrate (as is), 0.2% in total TMR. Also, phytase was added to the concentrate based on the respective dose level. Diets were provided to cows ad libitum, via sensor-controlled feeding troughs (Roughage-Intake Control, Insentec B.V., Marknesse, Netherlands) which measured the individual feed intake per cow per day. The TMR was supplied once daily, at 04:45, with a residual of approximately 5%. Feed refusals were removed and weighed daily.

The TMRs were formulated within the recommendations of the German Society of Nutrition and Physiology (GfE, 2001), except for P which was intentionally below the recommended P requirement (91.5%) in order to facilitate determination of whether the exogenous phytase would improve P digestibility. The chemical composition, feed values and phytase activity of the TMRs are shown in Table 8.

TABLE 8
Chemical composition, feed values
and phytase activity of the TMRs.

Dietary treatment¹

| | | | |
|---|---|---|---|
| Item | CON | PhyG2,000 | PhyG5,000 |

Chemical composition, g/kg dry matter

| | | | |
|---|---|---|---|
| Dry matter, g/kg | 356 | 346 | 360 |
| Ash | 70 | 68 | 68 |
| Crude protein | 167 | 158 | 167 |
| Crude fat | 32 | 34 | 34 |
| Neutral detergent fibre | 420 | 428 | 417 |
| Starch | 165 | 155 | 156 |
| Sugar | 45 | 39 | 41 |
| Calcium | 6.7 | 6.3 | 6.0 |
| Phosphorus (P) | 2.83 | 2.95 | 3.06 |
| Phytate-P | 0.86 | 0.83 | 0.83 |

Feed value²

| | | | |
|---|---|---|---|
| NEL (MJ/kg DM) | 7.00 | 7.00 | 7.00 |

Phytase activity, FTU/kg dry matter²

| | | | |
|---|---|---|---|
| Total phytase | 183 | 2284 | 7068 |
| Exogenous phytase | | 2101 | 6885 |

¹CON, control; PhyG2,000, containing PhyG phytase at 2,000 FTU/kg total ration on DM basis; PhyG5,000, containing PhyG phytase at 5,000 FTU/kg total ration on DM basis.
²Feed value of diets (net energy of lactation, NEL) estimated according to the German Society of Nutrition Physiology (GfE, 2001)
³Based on the analysis of phytase activity in concentrate and the % of concentrate in TMR on DM basis

Experimental design: The experiment was carried out as a randomized block design with three dietary treatments, 15 blocks (replicates) for the CON treatment and 16 blocks for the PhyG 2,000 and 5,000 treatments (47 cows in total). Within blocks, animals were randomly assigned to treatments. The experiment comprised a 14-day pre-period for data collection to facilitate allocation of cows to blocks based on parity, dry matter intake (DMI) and milk production. During this period, milk yield and feed intake data were collected daily and milk composition and analysis were recorded once per week. This was followed by a 19-day experimental period comprising a 14-d diet adaptation phase and 5 days of feces collection.

### Sampling, Measurements, and Chemical Analyses:

Milk: Cows were milked twice daily starting at 05:00 and 15:30 in a rapid exit milking parlour equipped on one side with a herringbone milking parlor for 8 cows and on the other with a side-by-side milking parlor for 10 cows (GEA Farm Technologies, Bönen, Germany). Milk samples were collected per week from each cow at the Monday afternoon and Tuesday morning milkings. Samples were preserved in bronopol (2-bromo-2-nitropropane-1,3-diol) for later analysis. Milk samples were analyzed for fat, protein, urea, lactose and somatic cell count (SCC) by infrared spectrophotometry using a MilkoScan FT6000 (Foss Aalytical A/S, Hillerød, Denmark). Two additional milk samples (50 mL) were taken per cow during the last week of the experiment on Monday evening and Tuesday morning, samples pooled (1:1, v:v) and stored at −20°, until later analysis of total P and calcium content. The samples were analyzed by an accredited laboratory by inductively coupled plasma optical emission spectrometry (ICP-OES) according to method VDLUFA III, 10.8.1.2, 2012 (DIN EN ISO 11885:1009-09).

Feed: The daily amounts of TMR offered and refused were recorded on an individual cow basis. The daily feed intake was calculated as the difference between the offered and refused amounts. Representative samples (500 g, fresh matter basis) of each forage (grass, corn, pulp silage) and of the TMRs were collected on days 3, 10, 13 and 14 of the experimental period. The forage samples were taken from the fresh cut-surface of the feed silos, pooled and mixed. Samples were vacuum sealed and frozen at −20 °C for later analysis. Samples of the concentrates (500 g) were separately taken from the feed silos on days 3 and 13 of the experimental period and every second day of the fecal sampling period. These were vacuum scaled and stored at −20 °C. An additional sample of each TMR was taken daily, as above, for the determination of dry matter content according to method VDLUFA III, 3.1 1976.

Frozen TMR and forage samples were crushed, mixed and divided into four samples of 500 g per forage or TMR. Frozen concentrates were pooled and divided into four 500 g samples. For analysis, samples were thawed, dried to constant weight at <60 °C in a drying oven (Memmert, Germany) and ground to pass through a 1 mm sieve using a ReschZM 200 grinder (Retch, Benelu, Aartselaar, Belgium). Ground samples were analyzed by LKS (Niederwiesa OT Lichtenwald, Germany) for the analysis of crude ash, crude protein, crude fat, sugar, starch, neutral detergent fiber (organic matter basis) according to the official methods of VDLUFA (VDLUFA, 2012) and in accordance with the general requirements of DIN EN ISO/IEC 17025:2018. Phosphorus and calcium were analyzed according to DIN EN ISO 11885:2009-09. Titanium dioxide was analyzed according to method DIN EN ISO 17294:2017-01 at SGS Analytics Germany GmbH (Jena, Germany). The net energy of lactation (NEL) of the diets was calculated according to the German Society of Nutrition Physiology (GfE, 2001). The phytate-P content of the diets was analyzed by Danisco Animal Nutrition Research Centre (Brabrand, Denmark) using the same methods used in Example 1. The phytase activity in feed was analyzed by the method described in International Patent Application Publication No. WO2020106796, incorporated by reference herein.

Feces: Fecal grab samples (~300-500 g of fecal matter per sample) were collected from all animals twice daily during the last five days of the experiment. Fecal sampling commenced at 09:00 and 13:00 on d 1, at 08:00 and 17:00 on day 2, at 10:00 and 17:00 on day 3, at 06:00 and 20:00 on day 4 and 06:00 and 13:00 on day 5. This sampling pattern was applied to account for diurnal and day-to-day variations in marker excretion (Glindemann et al., 2009). Samples were immediately frozen at −20 °C for later analysis. Prior to analysis, samples were thawed at room temperature, pooled per cow and freeze-dried for approximately 96 h in a freeze-dryer (Piatkowski, München, Germany) and ground to pass through a 1 mm sieve. Moisture, crude protein (CP), neutral detergent fiber (NDF), P, phytate-P and titanium dioxide in ground fecal samples were analyzed by the aforementioned laboratories using the aforementioned methods.

Blood: Blood samples were taken from each cow from the coccygeal vein on day 2 of the fecal collection period at approximately 10:00. Samples were allowed to clot for 45 min at room temperature (~19 °C) and then centrifuged at 1,900×g for 20 min at 4 °C to extract serum. Serum samples were stored at −20 °C until analysis for total P.

Body weight: Individual body weight (BW) was recorded twice daily directly after each milking. The BW was recorded via automatic weighing scale (GEA Farm Technologies, GmbH, Boenen, Germany).

### Sample Size, Calculations and Statistical Analysis:

The sample size calculation with the software G*Power (Version 3.1.9.7), based on the power-analysis as described by Cohen (1988). The Power analysis based on mean values of P digestibility and standard deviation with a confidence level of 95% and with a power of 0.7 to detect a statistically significant difference between treatments. The total sample size for 3 treatments is 46, meaning that replications should include 15 cows per treatment. In case a cow needed to be excluded from the study, e.g. due to illness, it was decided to utilize a replication of 16 cows per treatment group.

All data were averaged per cow and trial phase for the statistical analysis. The chemical composition of the total rations was calculated based on the chemical composition and intakes of both forages and concentrates. The intakes per day of nutrients and chemical components were calculated based on the individual dry matter intake (DMI) of each cow multiplied by the concentration of the respective nutrient or chemical component in the TMR. The apparent total tract digestibility (ATTD) of DM, ash, CP, NDF, P and phytate-P was calculated according to the following formula:

$$ATTD ( \% ) = \frac{{TiO}_{2 ( diet )}}{{TiO}_{2 ( fecces )}} \times \frac{{nutrient}_{( fecces )}}{{nutrient}_{( diet )}} ( \times 100 )$$

where TiO_{2(diet)} is the concentration of TiO₂ in the diet, TiO_{2(feces)} is the concentration of TiO₂ in the feces, TiO₂₍ₙᵤₜᵣᵢₑₙₜ₎ is the concentration of the nutrient or chemical constituent in the feces and nutrient (diet) is the concentration of the nutrient or chemical constituent in the diet.

The fecal excretion of CP, NDF, P, Ca, and phytate-P per cow per day was calculated according to the following formula:

Fecal excretion=nutrient intake−(ATTD %×nutrient intake)

The yield of FPCM (kg/d) was calculated on a 4% fat and 3.3% protein basis. The feed efficiency was calculated as FPCM (kg/d) divided by DMI (kg/d). Data on somatic cell count (SCC) were log transformed to obtain a normal distribution before statistical analysis.

All statistical analyses were performed using JMP (16.1). Data were analyzed by ANOVA to identify treatment effects. Block was included as a random effect. Data were checked for outliers before analysis. Treatment means comparisons were carried out using Tukey's HSD text. Statistical significance was declared at P<0.1.

Chemical composition of diets: In general, the chemical composition of the total rations (Table 8) was similar among treatments. The content of P was slightly higher than the targeted value (2.8 g/kg DM) in the two phytase-supplemented treatments, but the variation was less than 10%. Phytate-P levels in the concentrates were all above the targeted minimum of 2.3 g/kg. Phytase activity in the CON concentrate was low (508 FTU/kg, DM basis; Table 7) and was considered to reflect the presence of intrinsic (native) phytase present in the ingredients of the concentrate. The activity of exogenous phytase in the phytase-supplemented treatments were calculated as 2101 and 6885 FTU/kg DM TMR in PhyG2,000 and PhyG5,000, respectively (Table 8), indicating that the targeted phytase dose levels had been approximately achieved.

### Example 5: Feed Intake, Milk Yield and Composition, and Blood Analysis

The effect of PhyG supplementation on BW, feed intake, feed efficiency, milk yield, milk composition, SCC and blood analytes, with outliers removed from the dataset, is presented in Table 9.

TABLE 9
Effect of phytase supplementation on BW, dry matter intake,
feed efficiency, milk production and composition during the
19-d experimental period and 5-d fecal collection period

| | Dietary treatment¹ | | | | P- |
|---|---|---|---|---|---|
| Item | CON | PhyG2,000 | PhyG5,000 | SEM* | value |

5-d fecal collection period:
Dry matter intake, kg/d

| | | | | | |
|---|---|---|---|---|---|
| Total ration | 24.2 | 25.2 | 24.3 | 0.70 | 0.569 |

Milk production

| | | | | | |
|---|---|---|---|---|---|
| Milk, kg/d | 40.99 | 41.63 | 42.79 | 1.49 | 0.414 |
| FPCM², kg/d | 41.80 | 42.99 | 43.89 | 1.26 | 0.395 |
| Fat, kg/d | 1.70 | 1.79 | 1.76 | 0.06 | 0.531 |
| Protein, kg/d | 1.31^{b} | 1.40^{a} | 1.40^{a} | 0.03 | 0.041 |
| Lactose, kg/d | 2.00 | 2.04 | 2.10 | 0.07 | 0.335 |

Milk composition

| | | | | | |
|---|---|---|---|---|---|
| Fat, % | 4.26 | 4.27 | 4.13 | 0.11 | 0.503 |
| Protein, % | 3.30 | 3.33 | 3.31 | 0.08 | 0.895 |
| Lactose, % | 4.90 | 4.89 | 4.91 | 0.03 | 0.932 |
| Phosphorus, mg/100 mL | 114.4 | 114.3 | 112.8 | 2.23 | 0.810 |
| Calcium, mg/100 mL | 88.3 | 89.4 | 90.2 | 1.92 | 0.691 |
| Urea, mg/kg | 204.2 | 217.5 | 198.9 | 2.23 | 0.172 |
| Somatic cell count, Log₁₀, cells/ml | 2.37 | 2.71 | 1.93 | 0.45 | 0.329 |
| BW, kg | 747 | 759 | 748 | 17.07 | 0.791 |
| Feed efficiency³ | 1.75 | 1.73 | 1.82 | 0.07 | 0.552 |

Blood analytes

| | | | | | |
|---|---|---|---|---|---|
| Phosphorus, mmol/L | 1.48^{b} | 2.01^{a} | 1.80^{ab} | 0.12 | 0.015 |
| Phosphorus, mg/L | 45.8^{b} | 62.3^{a} | 55.9^{ab} | 3.62 | 0.015 |

19-d experimental period:
Dry matter intake, kg/d

| | | | | | |
|---|---|---|---|---|---|
| Total ration | 23.1 | 23.7 | 23.5 | 0.70 | 0.799 |

Milk production

| | | | | | |
|---|---|---|---|---|---|
| Milk, kg/d | 41.6 | 43.6 | 41.3 | 1.39 | 0.354 |
| FPCM², kg/d | 41.7 | 43.3 | 42.3 | 1.24 | 0.567 |
| Fat, kg/d | 1.70 | 1.73 | 1.74 | 0.05 | 0.838 |
| Protein, kg/d | 1.33^{b} | 1.42^{a} | 1.39^{ab} | 0.03 | 0.095 |
| Lactose, kg/d | 2.04 | 2.13 | 2.04 | 0.07 | 0.441 |

Milk composition

| | | | | | |
|---|---|---|---|---|---|
| Fat, % | 4.19 | 4.01 | 4.11 | 0.10 | 0.420 |
| Protein, % | 3.27 | 3.30 | 3.33 | 0.08 | 0.791 |
| Lactose, % | 4.89 | 4.89 | 4.92 | 0.03 | 0.729 |
| Urea, mg/kg | 198.3 | 193.4 | 195.8 | 7.20 | 0.900 |
| Somatic cell count, Log₁₀, cells/ml | 1.88 | 2.01 | 2.38 | 0.40 | 0.590 |
| BW, kg | 748.2 | 755.7 | 746.1 | 16.92 | 0.910 |
| Feed efficiency³ | 1.83 | 1.83 | 1.81 | 0.06 | 0.946 |

¹CON, control; PhyG2,000, containing PhyG phytase at 2,000 FTU/kg total ration on DM basis; PhyG5,000, containing PhyG phytase at 5,000 FTU/kg total ration on DM basis.
²Fat and protein corrected milk production (FPCM)
³Calculated as FPCM/DMI, both in kg
^{a,b}Means within a row bearing different upper case capital letters tended towards being significantly different at a level of P < 0.1.
*SEM is the mean of Std Error of the 3 treatments

Treatment had no effect on milk production, FPCM or milk composition, except for milk protein yield which was increased by phytase (at either dose level) during the 5-d fecal collection period (P<0.05). As milk protein synthesis is highly dependent on the availability of amino acids (AA), this is consistent with increased amino acid availability in the phytase-supplemented treatments due to the activity of the phytase in degrading phytate. Such an effect has been observed previously with PhyG in broilers and pigs (Dersjant-Li et al., 2022a). A tendency towards increased milk protein content was also evident during the entire 19-d experimental period (P=0.095). Body weight and feed efficiency were unaffected by treatment. PhyG2000 increased blood P concentration vs. CON (P<0.05), while PhyG5000 showed intermediate response.

### Example 6: Nutrient Intake, Excretion, and ATTD

The effect of treatment on nutrient intake, ATTD and fecal excretion during the 5-d fecal collection period, with outliers removed from the dataset, is presented in Table 10.

TABLE 10
Effect of phytase supplementation on nutrient intake, fecal excretion, and apparent
total tract digestibility (ATTD) during the 5-d fecal collection period

| | Dietary treatment¹ | | | | P- |
|---|---|---|---|---|---|
| Item | CON | PhyG2,000 | PhyG5,000 | SEM* | value |

Intake

| | | | | | |
|---|---|---|---|---|---|
| Dry matter, kg/d | 24.2 | 25.2 | 24.3 | 0.7 | 0.569 |
| Ash, kg/d | 1.7 | 1.71 | 1.65 | 0.05 | 0.669 |
| Crude protein, kg/d | 4.05 | 3.98 | 4.06 | 0.01 | 0.874 |
| Neutral detergent fiber, kg/d | 10.18 | 10.78 | 10.14 | 0.29 | 0.242 |
| Phosphorus (P), g/d | 72.69 | 75.56 | 72.92 | 2.09 | 0.569 |
| Phytate-P, g/d | 20.91 | 20.85 | 20.14 | 0.58 | 0.599 |

Fecal excretion

| | | | | | |
|---|---|---|---|---|---|
| Dry matter, kg/d | 9.97^{a} | 9.09^{ab} | 8.37^{b} | 0.375 | 0.009 |
| Ash, kg/d | 1.29^{a} | 1.15^{ab} | 1.01^{b} | 0.05 | 0.004 |
| Crude protein, kg/d | 1.75 | 1.69 | 1.58 | 0.07 | 0.224 |
| Neutral detergent fiber, kg/d | 5.21^{a} | 4.80^{ab} | 4.41^{b} | 0.2 | 0.011 |
| Phosphorus, g/d | 48.71^{a} | 44.79^{a} | 38.64^{b} | 2.14 | 0.004 |
| Phytate-P, g/d | 2.48^{a} | 0.80^{b} | 0.47^{b} | 0.12 | <.0001 |

ATTD, %

| | | | | | |
|---|---|---|---|---|---|
| Dry matter | 79.73 | 81.32 | 83.63 | 1.42 | 0.168 |
| Ash | 22.43^{b} | 32.87^{a} | 39.15^{a} | 2.77 | 0.001 |
| Crude protein | 57.05^{b} | 57.72^{ab} | 61.17^{a} | 1.33 | 0.087 |
| Neutral detergent fiber | 49.10^{b} | 55.56^{a} | 56.55^{a} | 1.5 | 0.002 |
| Phosphorus | 33.40^{b} | 40.79^{a} | 47.08^{a} | 2.53 | 0.001 |
| Phytate-P | 88.47^{b} | 96.15^{a} | 97.66^{a} | 0.56 | <.0001 |

¹CON, control; PhyG2,000, containing PhyG phytase at 2,000 FTU/kg; PhyG5,000, containing PhyG phytase at 5,000 FTU/kg.
^{a,b}Means bearing different superscript letters within a row are significantly different at P ≤ 0.10.
*SEM is the mean of Std Error of the 3 treatments

There were effects of treatment on fecal excretion of DM (P<0.01), ash (P<0.01), NDF (P<0.05), P (P<0.01) and phytate-P (P<0.001) and on ATTD of ash (P<0.01), NDF (P<0.01), P (P<0.01) and phytate-P (P<0.001). In all cases, fecal excretion of affected nutrients and dietary components was reduced in the phytase-supplemented treatments compared with CON. The numerical value of the treatment means suggested that these effects were dose-dependent. At a dose of 5,000 FTU/kg, PhyG reduced fecal excretion of P by 19.9%, phytate-P by 78.9% vs. CON, and the ATTD of CP was increased (P=0.087) by phytase. These results are consistent with the notion of the phytase having improved the proportion of digestible nutrients (in particular, phytate, NDF and protein) within the diet, leading to reduced nutrient excretion. This is consistent with the proven mode of action of the phytase in vitro and in vivo in pigs and poultry in rapidly and extensively degrading phytate (IP6) to low IP-esters (Christensen et al., 2020; Dersjant-Li et al., 2022c), Thereby releasing nutrients for digestion and absorption by the animal.

There was no effect of treatment on total DMI during the experimental period. Because of this, and the similar nutrient composition of the TMRs, there was also no effect of treatment on the intake of CP, NDF, Por phytate-P.

Conclusion from Examples 5 and 6: The results demonstrated a clear beneficial effect of PhyG supplementation on the milk protein yield and on fecal excretion of DM, ash, NDF, P and phytate-P, and ATTD of ash, CP, NDF, P and phytate-P, in mid-lactating dairy cows. ATTD of affected nutrients were all increased by PhyG, whereas fecal excretion of affected nutrients was decreased by PhyG. These results suggest that the phytase has potential to be used for improving P and protein availability in the diet whilst reducing their excretion and thereby the contribution to the environmental burden of dairy cow production.

### Example 7: Comparative Phytase Activity Analysis During Passage of the Ruminant Digestion System

This example describes an in-vitro study evaluating the effects on phytate hydrolysis occurring during passage of the ruminant digestion system upon addition of various commercial phytases. The model (2-step) included simulation of rumen and abomasum compartments under typical conditions found in-vivo. The in-vitro system employed was a modification of the Tilley and Terry method from 1963, which was further optimized specifically for evaluation of phytase performance in the rumen by Brask-Pedersen, et al., (2013).

Ruminal fluid was collected from 3 rumen-cannulated non-lactating dairy cows (Danish Holstein) maintained on a diet based on hay (grass and barley straw) and concentrate feed (oat, barley, soybean meal, rapeseed cake and beet molasses) comprised in a 68 to 32% DM basis ratio. Cows were fed 14 h prior to collection. The ruminal fluid including solids was collected from three cows on the morning of use, mixed in the proportion 1:1:1 (% v/v) and maintained at 38 to 39 °C (water bath) during transport. Ruminal fluid (pH 6.7) was then filtered through cheese cloth and subsequently mixed with a CO₂ degassed Bis-Tris buffer (0.036 M Bis-Tris, 0.12 M NaHCO₃, 0.008 M NaCl, 0.008M KCl, 0.0006M MgCl₂, 6H₂O, and 0.0004 M CaCl₂), 2H₂O) solution in the proportion 1:5. Finally, pH was adjusted to 6.2 (addition of 4 M HCl) at 40 °C. This solution will be referred to as the rumen fluid mixture.

Ground (Retsch mill, 0.7 mm sieve) rapeseed meal (0.60% IP6 P) was used as model substrate/feed. Rapeseed in general, has low endogenous phytase activity. The phytase activity of the specific rapeseed sample employed in this study was not detectable using a modified ISO 30024 method for analysis of phytase in feed (as described in WO2020106796, Example 5),

Three bacterial histidine acid phosphatase commercial phytases (applied in solid formulation): the phytase utilized in Example 1 (a.k.a. PhyG (IFF)), Ronozyme® HiPhos 20000GT (Novozymes/DSM), and Natuphos®) E 10000G, (BASF Nutrition) were evaluated along with a negative control containing rumen fluid mixture, but without exogenous phytase added. Phytases included in the study are all sold for commercial use in monogastric species. The phytases were dosed based on their phytase units (FTU) with 2FTU/g of substrate. Activities for standardization were obtained using a modified ISO 30024 method for analysis of phytase in products (as described in WO2020106796, Example 3, incorporated herein by reference).

For the timepoint incubations batches of 25 ml of rumen fluid mixture was portioned into 50 ml Falcon tubes already containing 0.5 g of rapeseed meal as substrate. The phytase treatments were added 1 ml of phytase stock solution (1.0 FTU/ml in demineralized water) to a final concentration of 2.0 FTU/g of rapeseed substrate. Controls were instead added 1 ml of demineralized water. Determinations for each treatment was done in duplicate (n=2) resulting in sets of 8 samples per incubation timepoint.

To maintain anaerobic conditions throughout the rumen incubation the Falcon tubes were again degassed with CO₂, capped (not tightened) and placed into a 2.5 L closed airtight container (Oxoid AnaeroJar, Thermo Scientific) containing Oxygen pads (Oxoid™ AnaeroGen, Thermo Scientific) and Anaerob indicator strips (Oxoid™ Resazurin, Thermo Scientific). Containers were then placed in an Incubator (Innova 40, New Brunswick Scientific) at a temperature of 40 °C and with continuous mixing at 50 rpm/min. Rumen incubation timepoints were 0, 3 and 6 h. After rumen incubation for 6 hours one set of samples was incubated for additionally 2 h at abomasum conditions. Abomasum simulations were carried out at a pH of 2.0 by addition of 1×1 ml of 1.1M HCl containing 50 mg pepsin/ml (Pepsin from porcine gastric mucosa, P7000, lot #BCCG9946 (>/=250 U/mg), 697 U/mg, Sigma).

To each rumen and abomasum timepoint one set of samples was removed from the incubator and phytase reaction stopped by addition of 2 ml of 37% HCl. For analysis of IP6 (High Performance Ion Exchange Chromatography) samples were first extracted on a rotating wheel for 1 h at room temperature and placed at −20 °C overnight. Upon thawing, extracts were centrifuged at 3000×g for 5 min and supernatant transferred to sterile filter devices (Millipore Ultrafree, Eppendorf) followed by ultra-centrifugal filter devices (Microcon YM-30, Millipore) and separated by centrifugation for 3000 and 12,000×g, 30 min, respectively. Flowthrough was transferred to 96-deepwell plates and analysed for IP6 according to the method described by Christensen et al., (2020).

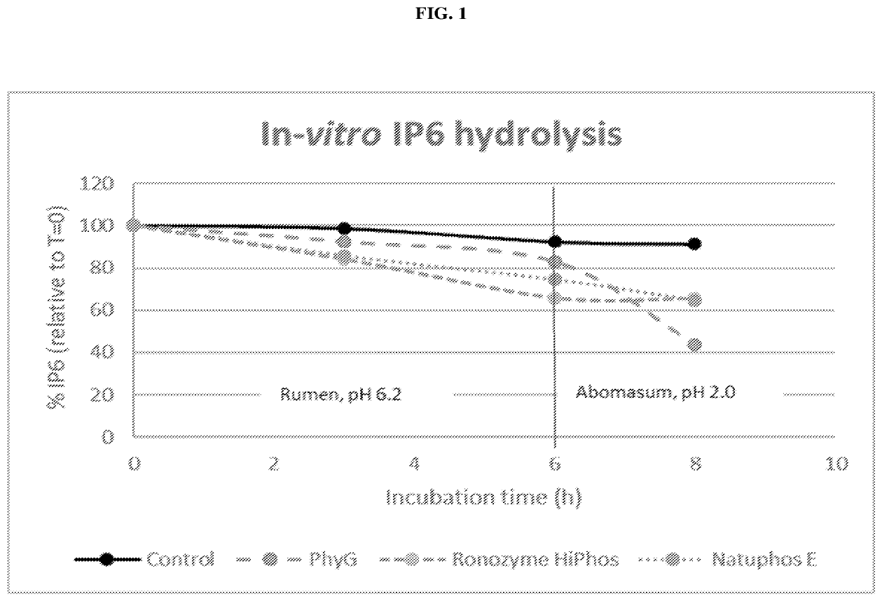

The results (FIG. 1) from the in-vitro evaluation show that all three phytase treatments resulted in higher IP6 hydrolysis after the rumen (6 h) and abomasum (2 h) simulations as compared to control (without phytase added). Some endogenous phytase activity originating from the microbial population of the rumen fluid resulted in 8% loss of IP6 (relative to t=0) over rumen incubation. PhyG degraded more IP6 as compared to the other phytases after total incubation (both rumen and abomasum) with only 44% remaining in contrast to the 65% (relative to t=0) for both Ronozyme® HiPhos and Natuphos® E. In the abomasum simulation, PhyG was able to degrade the IP6 at a much higher rate (from 83% after 6 h incubation in rumen to 44% after additional 2 h incubation in abomasum, meaning 47% reduction of IP6 remaining after rumen simulation), whereas Ronozyme® HiPhos (66% to 65%) and Natuphos® E (74% to 65%) had much less IP6 hydrolysis capability. In summary, PhyG was able to degrade around half the IP6 that entered abomasum in contrast to the two other phytases, which only hydrolyzed<15% of the remaining IP6 under abomasum conditions. An ideal exogenous phytase should be stable in the rumen and more active in the abomasum, in order to breakdown phytate or phytate-protein complex to make these and other (e.g. minerals) nutrients available for animals. Based on the in-vitro data, PhyG is more active in the abomasum and is able to release more P from phytate in this environment, which will lead to better P absorption. . . .

It can be concluded from these results, that PhyG more efficiently degrades IP6 in the abomasum environment at low pH and under presence of pepsin, resulting in greater total IP6 degradation compared to Ronozyme® HiPhos and Natuphos® E. Therefore, PhyG has potential to release more phosphate (resulting in improved phytate P-digestibility) that can be absorbed and utilized by dairy cows, and thereby supports important physiological processes, due to the more complete removal of IP6 eliminating its potential to act as an anti-nutritional factor.

### REFERENCES

Adedokun, et al., 2015. J. Anim. Sci. 93:168-175. doi.org/10.2527/jas.2014-7912
Amerah, et al., 2014. Poult. Sci. 93:906-915. doi.org/10.3382/ps.2013-03465
Armstrong & Beever, 1969. Proc. Nutr. Soc. 28:121-131. doi.org/10.1079/PNS19690023
Brask-Pedersen, et al., 2013. J. Dairy Sci. 96:1691-1700. doi.org/10.3168/jds.2011-5278
Bravo, et al., 2000. Reprod. Nutr. Dev. 40:149-162. doi.org/10.1051/rnd: 2000126
COMV, 2005. Mineral supply mannual, cattle, sheep, goats. Commissie Onderzoek Mierale Voeding. Handleiding Mineralenvoorziening Rundvee, Schapen, Geiten. 228 pp.
Christensen ct al., 2020. Curr. Biochem. Eng. 6:156-171.
Clark, et al., 1986. J. Dairy Sci. 69:3151-3155. doi.org/10.3168/JDS.S0022-0302% 2886%2980780-9
Cohen, J. 1988: Statistical power analysis for the behavioral science. Second edition, Lawrence erlbaum associates, publishers. ISBN 0-8058-0283-5.
Cowieson et al., 2004. Br. Poult. Sci. 45:101-109. doi.org/10.1080/00071660410001668923
CVB (Centraal Veevoederbureau), 2018. CVB Table Ruminants 2018. CVB.
Dersjant-Li et al., 2020a. Anim. Feed Sci. Technol. 264:114481. doi.org/10.1016/j.anifeedsci.2020.114481
Dersjant-Li et al., 2020b. Anim. Nutr. 6:14-30. doi.org/10.1016/j.aninu.2021.11.002
Dersjant-Li et al., 2022a. J. Anim. Sci. 100:1-9. doi.org/10.1093/jas/skac037
Dersjant-Li et al., 2022b. Poult. Sci. 101:101666. doi.org/10.1016/j.psj.2021.101666
Dersjant-Li et al., 2022c. Brit. Poult. Sci. 63:395-405. doi.org/10.1080/00071668.2021.2000586
DIN EN ISO 2009. Water quality—Determination of selected elements by inductively coupled plasma optical emession spectrometry (ICP-OES).
DIN EN ISO 2017. Water quality—Application of inductively coupled plasma mass spectrometry (ICP-MS)—Part 2: Determination of selected elements including uranium isotopes (ISO 17294-2:2016), German version.
DIN EN ISO 2018. General requirements for the competence of testing and calibration laboratories (ISO/IEC 17025:2017); German version.
Edmonson et al., 1989. J. Dairy Sci. 72:68-78. doi.org/10.3168/jds.S0022-0302 (89) 79081-0
Engelen, et al., 2001. J. AOAC Int. 84:629-633. doi.org/10.1093/jaoac/84.3.629
Englyst et al., 1992. Classification and measurement of nutritionally important starch fractions. Eur. J. Clin. Nutr. 46 (Suppl 2): S33-50.
Forar et al., 1982. J. Dairy Sci. 65:760-763. doi.org.10.3168/jds.S0022-0302 (82) 82264-9
GfE, 1991. Leitlinien fur die Bestimmung der Verdaulichkeit von Rohneahrstoffen an Wiederkauern. J. Anim. Physiol. Anim. Nutr. 65:229-234.
GfE (Gesellschaft fur Ernahrungsphysiologie) 2001: Empfehlungen zur Energie-und Nahrstoffversorgung der Milchkuee yhe und Aufzuchtrinder. DLG-Verlag, Frankfurt a. M.
Giagnoni et al., 2021. Anim. Feed Sci. Technol. 276:114913. doi.org/10.1016/j.anifeedsci.2021.114913.
Glindemann et al., 2009. Feed Sci. Technol. 152:186-197. doi.org/10.3168/jds.2017-13283
Guyton et al., 2003. J. Dairy Sci. 86:3972-3982. doi.org/10.3168/jds.S0022-0302 (03) 74008-9
Haese, et al., 2020. Animal 14:1461-1471. doi.org/10.1017/S1751731120000221
Humer et al., 2015. J. Anim. Physiol. Anim. Nutr. (Berl) 99:605-625. doi.org/10.1111/jpn.12258
Humer and Zebeli, 2015. Anim. Feed Sci. Technol. 209:1-15. doi.org/10.1016/j.anifeedsci.2015.07.028
ISO, 1998. ISO 6491: Animal feeding stuffs—Determination of phosphorus content—Spectrometric method. Retrieved from iso.org/standard/12864.html
ISO, 2000. ISO 6869: Animal feeding stuffs—Determination of the contents of calcium, copper, ion, magnesium, manganese, potassium, sodium and zinc—Method using atomic absorption spectrometry. iso.org/standard/33707.html
ISO, 2002. ISO 5984: Animal feeding stuffs—determination of crude ash. iso.org/standard/37272.html
ISO, 2006. ISO, 16472: Animal feeding stuffs—Determination of amylase-treated neutral detergent fibre content (aNDF). iso.org/standard/37898.html
ISO, 2008. ISO 13906: Animal feeding stuffs—Determination of acid detergent fibre (ADF) and acid detergent lignin (ADL) contents. iso.org/standard/43032.html
ISO, 2015. ISO 11085: Cereals, cereals-based products and animal feeding stuffs—Determination of crude fat content by the Randall extraction method. iso.org/standard/63542.html
ISO, 2016. ISO 16634: Food products—Determination of the total nitrogen content by combustion according to the Dumas principle and calculation of the crude protein content—Part 2: Cereals, pulses and milled cereal products. iso.org/standard/66661.html
Jarrett et al., 2014. J. Dairy Sci. 97:411-418. doi.org/10.3168/jds.2013-7124
Kim et al., 2018. BMC Vet. Res. 14:310. doi.org/10.1186/s12917-018-1637-3
Kim & Lee, 2012. Animals 11:2118. doi.org/10.3390% 2Fani11072118
Kincaid et al., 2005. J. Dairy Sci. 88:2893-2902. doi.org/10.3168/jds.S0022-0302 (05) 72970-2
Knowlton et al., 2007. J. Dairy Sci. 90:4356-4360. doi.org/10.3168/jds.2006-879
Krämer & Weisbjerg. 2013. J. Dairy Sci. 96:3163-3176. doi.org/10.3168/jds.2012-6146
Liu et al., 2008. J. Anim. Sci. 86:3432-3439. doi.org/10.2527/jas.2008-1234
Morse et al., 1992. J. Dairy Sci. 75:1979-1986. doi.org/10.3168/jds.s0022-0302 (92) 77957-0
NRC. 2001. Nutrient requirements of dairy cattle, seventh rev. ed. Natl. Acad. Sci., Washington, DC.
Puhl et al., 2008. Int. J. Biochem. Cell Biol. 40:2053-2064. doi.org/10.1016/j.biocel.2008.02.003
Raun et al., 1956. J. Agric. Food Chem. 4:869-871. doi.org/10.1021/jf60068a006
Ravindran et al., 2006. Poult. Sci. 85:82-89. doi.org/10.1093/ps/85.1.82
Read et al., 1986. S. Afr. J. Anim. Sci. 16:18-22.
Selle et al., 2000. Nutr. Res. Rev. 13:255-278. doi.org/10.1079/095442200108729098
Selle & Ravindran, 2007. Anim. Feed Sci. Technol. 135:1-41. doi.org/10.1016/j.anifeedsci.2006.06.010
Selle et al., 2009. Livest. Sci. 124:126-141. doi.org/10.1016/j.livsci.2009.01.006
Selle et al., 2012. Nutr. Res. Rev. 25:1-17. doi.org/10.1017/S0954422411000151
Sharpley et al., 1994. J. Environ. Qual. 23:437-451. doi.org/10.2134/jeq1994.00472425002300030006x
Skoglund et al., 1998. J. Agric. Food Chem. 46:1877-1882. doi.org/10.1021/jf9709257
Tamminga & Verstegen, 1992. Implications of nutrition of animals on environmental pollution Pages 113-129 in Recent advances in animal nutrition. P. C. Garnsworthy ed. Butterworth/Heinemann, London.
Taylor et al., 2001. Proceedings, Western Section, American Society of Animal Science 52.
TILLEY, J. M. A. and TERRY, R. A. 1963. J. Br. Grassl. Soc. 18:104-1 1 1.
Truong et al., 2014. Anim. Feed Sci. Technol. 198:248-256. doi.org/10.1016/j.anifeedsci.2014.10.007
Truong et al., 2015. Anim. Feed Sci. Technol. 209:240-248. doi.org/10.1016/j.anifeedsci.2015.08.012
Valk, et al., 2000. J. Environ. Qual. 29:28-36. doi.org/10.2134/jeq 2000.00472425002900010005x
VDLUFA (Association of German Agricultural Analytic and Research Institutes) 2012. Method book III “The chemical analysis of feedstuffs” of VDLUFA (1st-7th supplement delivery).
Winter et al., 2015. Ital. J. Anim. Sci. 14:53-60. doi.org/10.4081/ijas.2015.3539
Wu et al., 2003. J. Dairy Sci. 86:3300-3308. doi.org/10.3168/jds.S0022-0302 (03) 73931
Yanke et al., 1998. Microbiology 144:1565-1573. doi.org/10.1099/00221287-144-6-1565

We claim:

1. A method for improving phosphorous and/or protein utilization in a lactating ruminant animal comprising administering to the animal a phytase polypeptide or a fragment thereof comprising phytase activity.

2. The method of claim 1, wherein the phytase polypeptide or a fragment thereof comprises at least 82% sequence identity with the amino acid sequence set forth in SEQ ID NO:1.

3. The method of claim 1 or claim 2, wherein improving protein utilization comprises a) improved digestibility and/or decreased excretion of crude protein; and/or b) improved milk protein content and protein yield.

4. The method of any one of claims 1-3, wherein improved phosphorous utilization comprises improved digestibility and/or decreased excretion of total phosphorus and/or phytate bound phosphorous.

5. The method of any one of claims 1-4, wherein the method further improves calcium digestibility.

6. The method of any one of claims 1-4, wherein the lactating ruminant animal is selected from the group consisting of cattle, young calves, goats, sheep, giraffes, bison, moose, elk, yaks, water buffalo, deer, reindeer, caribou, camels, alpacas, llamas, antelope, pronghorn and nilgai.

7. The method of any one of claims 1-5, wherein the phytase polypeptide or a fragment thereof comprising phytase activity is not coated.

8. The method of any one of claims 1-5, wherein the phytase polypeptide or a fragment thereof comprising phytase activity is formulated for bypassing the rumen.

9. The method according to any one of claims 1-6, wherein the phytase polypeptide or a fragment thereof comprising phytase activity is administered in combination with a feed.

10. The method of claim 9, wherein the feed is selected from the group consisting of total mixed ration (TMR), compound feed, mineral premix.

11. The method of claim 9, wherein the feed comprises one or more cereal byproducts of a distillation process.

12. The method of claim 11, wherein the phytase polypeptide or a fragment thereof comprising phytase activity is used during a saccharification and/or fermentation reaction prior to the distillation process.

13. The method of any one of claims 9-12, wherein the feed comprises one or more of corn gluten meal, Distillers Dried Grains with Solubles (DDGS), corn based Distillers Dried Grains with Solubles (cDDGS), wheat bran, wheat middlings, wheat shorts, rice bran, rice hulls, oat hulls, palm kernel, or citrus pulp.

14. The method of claim 10, wherein the mineral premix is administered as a licking block.

15. The method according to any one of claims 1-6, wherein the phytase polypeptide or a fragment thereof comprising phytase activity is administered in a water line.

16. The method of any one of claims 1-7, further comprising administering at least one additional enzyme to the animal.

17. The method of any one of claims 1-16, wherein the phytase polypeptide is administered in conjunction with a diet having low phosphorous content.

18. The method of any one of claims 1-17, wherein the phytase polypeptide or a fragment thereof comprises SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO:15, SEQ ID NO:16, SEQ ID NO: 17, SEQ ID NO:18, SEQ ID NO: 19, SEQ ID NO:20, SEQ ID NO:21, SEQ ID NO:22, SEQ ID NO: 23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO: 34, SEQ ID NO:35, SEQ ID NO:36, or SEQ ID NO:37.

19. A method for decreasing phosphate and/or nitrogen excretion in the feces of a lactating ruminant animal comprising administering to the animal a phytase polypeptide or a fragment thereof comprising phytase activity.

20. The method of claim 19, wherein the phytase polypeptide or a fragment thereof comprises at least 82% sequence identity with the amino acid sequence set forth in SEQ ID NO: 1.

21. The method of claim 16 or claim 20, wherein the lactating ruminant animal is selected from the group consisting of cattle, young calves, goats, sheep, giraffes, bison, moose, elk, yaks, water buffalo, deer, reindeer, caribou, camels, alpacas, llamas, antelope, pronghorn and nilgai.

22. The method of any one of claims 19-21, wherein the phytase polypeptide or a fragment thereof comprising phytase activity is not coated.

23. The method of any one of claims 19-22, wherein the phytase polypeptide or a fragment thereof comprising phytase activity is formulated for bypassing the rumen.

24. The method according to any one of claims 19-23, wherein the phytase polypeptide or a fragment thereof comprising phytase activity is administered in combination with a feed.

25. The method of claim 24, wherein the feed is selected from the group consisting of total mixed ration (TMR), compound feed, mineral premix.

26. The method of claim 24, wherein the feed comprises one or more cereal byproducts of a distillation process.

27. The method of claim 26, wherein the phytase polypeptide or a fragment thereof comprising phytase activity is used during a saccharification and/or fermentation reaction prior to the distillation process.

28. The method of any one of claims 24-27, wherein the feed comprises one or more of corn gluten meal, Distillers Dried Grains with Solubles (DDGS), corn-based Distillers Dried Grains with Solubles (cDDGS), wheat bran, wheat middlings, wheat shorts, rice bran, rice hulls, oat hulls, palm kernel, or citrus pulp.

29. The method of claim 25, wherein the mineral premix is administered as a licking block.

30. The method according to any one of claims 19-21, wherein the phytase polypeptide or a fragment thereof comprising phytase activity is administered in a water line.

31. The method of any one of claims 19-22, further comprising administering at least one additional enzyme to the animal.

32. The method of any one of claims 19-31, wherein the phytase polypeptide is administered in conjunction with a diet having low phosphorous content.

33. The method of any one of claims 19-32, wherein the phytase polypeptide or a fragment thereof comprises SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO:15, SEQ ID NO:16, SEQ ID NO: 17, SEQ ID NO:18, SEQ ID NO: 19, SEQ ID NO:20, SEQ ID NO:21, SEQ ID NO:22, SEQ ID NO: 23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO: 34, SEQ ID NO:35, SEQ ID NO:36, or SEQ ID NO:37.

34. A method for degrading phytate in the abomasum of a ruminant animal comprising administering to the animal a phytase polypeptide or a fragment thereof comprising at least 82% sequence identity of the amino acid sequence set forth in SEQ ID NO:1.

35. The method of claim 34, wherein the phytase polypeptide or a fragment thereof degrades phytate in the abomasum to a greater extent as compared to a phytase polypeptide or a fragment thereof that does not comprise at least 82% sequence identity of the amino acid sequence set forth in SEQ ID NO:1.

36. The method of claim 34 or claim 35, wherein the lactating ruminant animal is selected from the group consisting of cattle, young calves, goats, sheep, giraffes, bison, moose, elk, yaks, water buffalo, deer, reindeer, caribou, camels, alpacas, llamas, antelope, pronghorn and nilgai.

37. The method of any one of claims 34-36, wherein the phytase polypeptide or a fragment thereof comprising phytase activity is not coated.

38. The method according to any one of claims 34-37, wherein the phytase polypeptide or a fragment thereof comprising phytase activity is administered in combination with a fecd.

39. The method of claim 38, wherein the feed is selected from the group consisting of total mixed ration (TMR), compound feed, mineral premix.

40. The method of claim 38, wherein the feed comprises one or more cereal byproducts of a distillation process.

41. The method of claim 40, wherein the phytase polypeptide or a fragment thereof comprising phytase activity is used during a saccharification and/or fermentation reaction prior to the distillation process.

42. The method of any one of claims 38-41, wherein the feed comprises one or more of corn gluten meal, Distillers Dried Grains with Solubles (DDGS), corn based Distillers Dried Grains with Solubles (cDDGS), wheat bran, wheat middlings, wheat shorts, rice bran, rice hulls, oat hulls, palm kernel, or citrus pulp.

43. The method of claim 39, wherein the mineral premix is administered as a licking block.

44. The method according to any one of claims 34-37, wherein the phytase polypeptide or a fragment thereof comprising phytase activity is administered in a water line.

45. The method of any one of claims 34-37, further comprising administering at least one additional enzyme to the animal.

46. The method of any one of claims 334-45, wherein the phytase polypeptide is administered in conjunction with a diet having low phosphorous content.

47. The method of any one of claims 34-46, wherein the phytase polypeptide or a fragment thereof comprises SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, SEQ ID NO:11, SEQ ID NO:12, SEQ ID NO:13, SEQ ID NO:14, SEQ ID NO:15, SEQ ID NO: 16, SEQ ID NO: 17, SEQ ID NO:18, SEQ ID NO:19, SEQ ID NO:20, SEQ ID NO:21, SEQ ID NO:22, SEQ ID NO: 23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO: 34, SEQ ID NO:35, SEQ ID NO:36, or SEQ ID NO:37.
